SEQUENCE LISTING (146 sequences)

SEQ ID NO: 1 - protein, 176 aa, synthetic construct
  REGION 1..176: note = Synthetic Sequence_Amino acid sequence of his-tagged human TSLP antigen (huTSLP-his)
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKARKSKVTTNKCLEQVSQLQGLWRRFNRPLLKQQGSSDYKDDDDKHHHHHH

SEQ ID NO: 2 - protein, 398 aa, synthetic construct
  REGION 1..398: note = Synthetic Sequence_Amino acid sequence of Fc-tagged human TSLP antigen (huTSLP-Fc)
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKARKSKVTTNKCLEQVSQLQGLWRRFNRPLLKQQDIEGRMDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 168 aa, synthetic construct
  REGION 1..168: note = Synthetic Sequence_Amino acid sequence of his-tagged cyno TSLP antigen (cynoTSLP-his)
METDTLLLWVLLLWVPGSTGYDFTNCDFQKIEADYLRTISKDLITYMSGTKSTDFNNTVSCSNRPHCLTEIQSLTFNPTPRCASLAKEMFARKTKATLALWCPGYSETQINATQAMKKARKSKVTTNKCLEQVSQLLGLWRRFIRTLLKQQGSSDYKDDDDKHHHHHH

SEQ ID NO: 4 - protein, 390 aa, synthetic construct
  REGION 1..390: note = Synthetic Sequence_Amino acid sequence of Fc-tagged cyno TSLP antigen (cyno TSLP-Fc)
METDTLLLWVLLLWVPGSTGYDFTNCDFQKIEADYLRTISKDLITYMSGTKSTDFNNTVSCSNRPHCLTEIQSLTFNPTPRCASLAKEMFARKTKATLALWCPGYSETQINATQAMKKARKSKVTTNKCLEQVSQLLGLWRRFIRTLLKQQDIEGRMDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 446 aa, synthetic construct
  REGION 1..446: note = Synthetic Sequence_Amino acid sequence of Fc-tagged human TSLP receptor extracellular domain (human-TSLPR-Fc-ECD)
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKDIEGRMDEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 121 aa, Mus musculus
EVQLQQSGPVLVKPGASVKMSCKASGYTFTDDYMNWVKQSHGKSLEWIGIISPYNGGTSYNQKFKGKATLTVDKSSSTAYMELNSLTSEDSAVYYCAREDYDYDGYAMDHWGQGTSVTVSS

SEQ ID NO: 7 - protein, 105 aa, Mus musculus
QIVLSQSPAILSASPGEKVTMTCRASSSVSYMHWYQQKPGSSPKPWIYATSNLASGVPARFSGSGSGTSYSLTISRVEAEDAATYYCQQWSSNRTFGGGTKLEIK

SEQ ID NO: 8 - protein, 120 aa, Mus musculus
QAYLQQSGAELVRPGASVKMSCKASGFAFTTYNMHWVKHTPGQGLEWIGAIYPGNGETSYNQKFKDRATLTVDKSSRTAYMQLSSLTSEDSAVYFCAREDDYGEGYFDVWGAGTTVTVSS

SEQ ID NO: 9 - protein, 111 aa, Mus musculus
DIVLTQSPASLAVSLGQRATISCRASESVDNSGLSFMHWYQQKPGQPPRLLLYRASNLGSGIPARFSGSGSGTDFTLTLNPVETDDVATYYCQQINTDPLTFGAGTKLELK

SEQ ID NO: 10 - protein, 119 aa, Mus musculus
QVQLQQSGAELVRPGTSVKVSCKASGYAFTNYLIEWVKQRPGQGLEWIGVIDPGNGDTNYNENFKGKATLTADKSSSTAYIELSRLTSEDSAVYFCAREDNTGTAFDYWGQGTTLTVSS

SEQ ID NO: 11 - protein, 107 aa, Mus musculus
SIVMTQTPKFLLVSAGDRVTISCKASQSVSSDVTWYQQKPGQSPKLLIYYVSNHYTGVPDRFTGSGYGTDFTFTISSVQAEDLAVYFCQQHHRFPLTFGAGTKLELK

SEQ ID NO: 12 - protein, 117 aa, Mus musculus
QVQLQQSGPQLVRPGASVKISCKASGYSFTTYWMHWVKQRPGQGLEWIGMIDPSDSETTLIQKFKDKATLTVDKSSSTAYMQLSSPTSEDSAVYYCARTLDGYYDYWGQGTTLTVSS

SEQ ID NO: 13 - protein, 107 aa, Mus musculus
DIQMTQSPASLSASVGETVTITCRASENIYSYLAWYQQKQGKSPQLLVYFAKTLAEGVPSRFSGSVSGTQFSLKINSLQPEDFGSYYCQHHYGTPWTFGGGTKLEIK

SEQ ID NO: 14 - protein, 5 aa, Mus musculus
DDYMN

SEQ ID NO: 15 - protein, 17 aa, Mus musculus
IISPYNGGTSYNQKFKG

SEQ ID NO: 16 - protein, 12 aa, Mus musculus
EDYDYDGYAMDH

SEQ ID NO: 17 - protein, 10 aa, Mus musculus
RASSSVSYMH

SEQ ID NO: 18 - protein, 7 aa, Mus musculus
ATSNLAS

SEQ ID NO: 19 - protein, 8 aa, Mus musculus
QQWSSNRT

SEQ ID NO: 20 - protein, 5 aa, Mus musculus
TYNMH

SEQ ID NO: 21 - protein, 17 aa, Mus musculus
AIYPGNGETSYNQKFKD

SEQ ID NO: 22 - protein, 11 aa, Mus musculus
EDDYGEGYFDV

SEQ ID NO: 23 - protein, 15 aa, Mus musculus
RASESVDNSGLSFMH

SEQ ID NO: 24 - protein, 7 aa, Mus musculus
RASNLGS

SEQ ID NO: 25 - protein, 9 aa, Mus musculus
QQINTDPLT

SEQ ID NO: 26 - protein, 5 aa, Mus musculus
NYLIE

SEQ ID NO: 27 - protein, 17 aa, Mus musculus
VIDPGNGDTNYNENFKG

SEQ ID NO: 28 - protein, 10 aa, Mus musculus
EDNTGTAFDY

SEQ ID NO: 29 - protein, 11 aa, Mus musculus
KASQSVSSDVT

SEQ ID NO: 30 - protein, 7 aa, Mus musculus
YVSNHYT

SEQ ID NO: 31 - protein, 9 aa, Mus musculus
QQHHRFPLT

SEQ ID NO: 32 - protein, 5 aa, Mus musculus
TYWMH

SEQ ID NO: 33 - protein, 17 aa, Mus musculus
MIDPSDSETTLIQKFKD

SEQ ID NO: 34 - protein, 8 aa, Mus musculus
TLDGYYDY

SEQ ID NO: 35 - protein, 11 aa, Mus musculus
RASENIYSYLA

SEQ ID NO: 36 - protein, 7 aa, Mus musculus
FAKTLAE

SEQ ID NO: 37 - protein, 9 aa, Mus musculus
QHHYGTPWT

SEQ ID NO: 38 - protein, 105 aa, synthetic construct
  REGION 1..105: note = Synthetic Sequence_hu3 VL-CDR grafted
EIVLTQSPATLSLSPGERATLSCRASSSVSYMHWYQQKPGQAPRLLIYATSNLASGIPARFSGSGSGTDFTLTISRLEPEDFAVYYCQQWSSNRTFGGGTKVEIK

SEQ ID NO: 39 - protein, 105 aa, synthetic construct
  REGION 1..105: note = Synthetic Sequence_hu3VL2
EIVLTQSPATLSLSPGERATLSCRASSSVSYMHWYQQKPGQAPRPLIYATSNLASGIPARFSGSGSGTDYTLTISRLEPEDFAVYYCQQWSSNRTFGGGTKVEIK

SEQ ID NO: 40 - protein, 105 aa, synthetic construct
  REGION 1..105: note = Synthetic Sequence_hu3VL3
EIVLTQSPATLSLSPGERATLSCRASSSVSYMHWYQQKPGQAPRPWIYATSNLASGVPARFSGSGSGTDYTLTISRLEPEDFAVYYCQQWSSNRTFGGGTKVEIK

SEQ ID NO: 41 - protein, 105 aa, synthetic construct
  REGION 1..105: note = Synthetic Sequence_hu3VL4
EIVLTQSPATLSLSPGERATLSCRASSSVSYMHWYQQKPGQAPRPWIYATSNLASGVPARFSGSGSGTSYTLTISRLEPEDFAVYYCQQWSSNRTFGGGTKVEIK

SEQ ID NO: 42 - protein, 121 aa, synthetic construct
  REGION 1..121: note = Synthetic Sequence_hu3VH1
EVQLVQSGAEVKKPGASVKVSCKASGYTFTDDYMNWVRQAPGQRLEWMGIISPYNGGTSYNQKFKGRVTITRDTSASTAYMELSSLRSEDTAVYYCAREDYDYDGYAMDHWGQGTTVTVSS

SEQ ID NO: 43 - protein, 121 aa, synthetic construct
  REGION 1..121: note = Synthetic Sequence_hu3VH2
EVQLVQSGAEVKKPGASVKVSCKASGYTFTDDYMNWVRQAPGQRLEWMGIISPYNGGTSYNQKFKGRVTLTVDKSASTAYMELSSLRSEDTAVYYCAREDYDYDGYAMDHWGQGTTVTVSS

SEQ ID NO: 44 - protein, 121 aa, synthetic construct
  REGION 1..121: note = Synthetic Sequence_hu3VH3
EVQLVQSGAEVKKPGASVKVSCKASGYTFTDDYMNWVKQAPGQRLEWIGIISPYNGGTSYNQKFKGRATLTVDKSASTAYMELSSLRSEDTAVYYCAREDYDYDGYAMDHWGQGTTVTVSS

SEQ ID NO: 45 - protein, 12 aa, synthetic construct
  REGION 1..12: note = Synthetic Sequence_hu3 HCDR3-H110Y
EDYDYDGYAMDY

SEQ ID NO: 46 - protein, 8 aa, synthetic construct
  REGION 1..8: note = Synthetic Sequence_hu3LCDR3-N93D
QQWSSDRT

SEQ ID NO: 47 - protein, 12 aa, synthetic construct
  REGION 1..12: note = Synthetic Sequence_hu3 HCDR3 (general formula)
  VARIANT 12: note = H or Y
EDYDYDGYAMDX

SEQ ID NO: 48 - protein, 8 aa, synthetic construct
  REGION 1..8: note = Synthetic Sequence_hu3 LCDR (general formula 1)
  VARIANT 6: note = N or D
QQWSSXRT

SEQ ID NO: 49 - protein, 105 aa, synthetic construct
  REGION 1..105: note = Synthetic Sequence_Light chain variable sequence of hu3-11
EIVLTQSPATLSLSPGERATLSCRASSSVSYMHWYQQKPGQAPRPWIYATSNLASGVPARFSGSGSGTSYTLTISRLEPEDFAVYYCQQWSSDRTFGGGTKVEIK

SEQ ID NO: 50 - protein, 121 aa, synthetic construct
  REGION 1..121: note = Synthetic Sequence_Heavy chain variable sequence of hu3-11
EVQLVQSGAEVKKPGASVKVSCKASGYTFTDDYMNWVRQAPGQRLEWMGIISPYNGGTSYNQKFKGRVTLTVDKSASTAYMELSSLRSEDTAVYYCAREDYDYDGYAMDYWGQGTTVTVSS

SEQ ID NO: 51 - protein, 105 aa, synthetic construct
  REGION 1..105: note = Synthetic Sequence_hu3VL5
EIVLTQSPATLSLSPGERATLSCRASSSVSYMHWYQQKPGQAPRPWIYATSNLASGVPARFSGSGSGTSYTLTISRLEPEDFAVYYCQQSDNVRGFGGGTKVEIK

SEQ ID NO: 52 - protein, 105 aa, synthetic construct
  REGION 1..105: note = Synthetic Sequence_hu3VL6
EIVLTQSPATLSLSPGERATLSCRASSSVSYMHWYQQKPGQAPRPWIYATSNLASGVPARFSGSGSGTSYTLTISRLEPEDFAVYYCQQSDSGREFGGGTKVEIK

SEQ ID NO: 53 - protein, 8 aa, synthetic construct
  REGION 1..8: note = Synthetic Sequence_hu3 LCDR3-V1
QQSDNVRG

SEQ ID NO: 54 - protein, 8 aa, synthetic construct
  REGION 1..8: note = Synthetic Sequence_hu3 LCDR3-V2
QQSDSGRE

SEQ ID NO: 55 - protein, 8 aa, synthetic construct
  REGION 1..8: note = Synthetic Sequence_hu3 LCDR3 (general formula 2)
  VARIANT 5: note = N or S
  VARIANT 6: note = V or G
  VARIANT 8: note = G or E
QQSDXXRX

SEQ ID NO: 56 - protein, 111 aa, synthetic construct
  REGION 1..111: note = Synthetic Sequence_hu119-VL CDR grafted(IGKV4-1*01)
DIVMTQSPDSLAVSLGERATINCRASESVDNSGLSFMHWYQQKPGQPPKLLIYRASNLGSGVPDRFSGSGSGTDFTLTISSLQAEDVAVYYCQQINTDPLTFGQGTKLEIK

SEQ ID NO: 57 - protein, 111 aa, synthetic construct
  REGION 1..111: note = Synthetic Sequence_hu119VL2
DIVLTQSPDSLAVSLGERATINCRASESVDNSGLSFMHWYQQKPGQPPKLLIYRASNLGSGVPDRFSGSGSGTDFTLTISSLQAEDVAVYYCQQINTDPLTFGQGTKLEIK

SEQ ID NO: 58 - protein, 111 aa, synthetic construct
  REGION 1..111: note = Synthetic Sequence_hu119VL3
DIVMTQSPDSLAVSLGERATINCRASESVDNSGLSFMHWYQQKPGQPPKLLLYRASNLGSGIPDRFSGSGSGTDFTLTISSLQAEDVAVYYCQQINTDPLTFGQGTKLEIK

SEQ ID NO: 59 - protein, 111 aa, synthetic construct
  REGION 1..111: note = Synthetic Sequence_hu119VL4 (Grafted, IGKV3-11*01)
EIVLTQSPATLSLSPGERATLSCRASESVDNSGLSFMHWYQQKPGQAPRLLIYRASNLGSGIPARFSGSGSGTDFTLTISSLEPEDFAVYYCQQINTDPLTFGQGTKLEIK

SEQ ID NO: 60 - protein, 111 aa, synthetic construct
  REGION 1..111: note = Synthetic Sequence_hu119VL5
EIVLTQSPATLSLSPGERATLSCRASESVDNSGLSFMHWYQQKPGQPPRLLLYRASNLGSGIPARFSGSGSGTDFTLTISSLEPEDFAVYYCQQINTDPLTFGQGTKLEIK

SEQ ID NO: 61 - protein, 111 aa, synthetic construct
  REGION 1..111: note = Synthetic Sequence_hu119VL6
DIVLTQSPATLSLSPGERATLSCRASESVDNSGLSFMHWYQQKPGQPPRLLLYRASNLGSGIPARFSGSGSGTDFTLTISSLEPEDFAVYYCQQINTDPLTFGQGTKLEIK

SEQ ID NO: 62 - protein, 120 aa, synthetic construct
  REGION 1..120: note = Synthetic Sequence_hu119VH1 (Grafted)
EVQLVQSGAEVKKPGSSVKVSCKASGGTFSTYNMHWVRQAPGQGLEWMGAIYPGNGETSYNQKFKDRVTITADKSTSTAYMELSSLRSEDTAVYYCAREDDYGEGYFDVWGQGTTVTVSS

SEQ ID NO: 63 - protein, 120 aa, synthetic construct
  REGION 1..120: note = Synthetic Sequence_hu119VH2
EVQLVQSGAEVKKPGSSVKVSCKASGFTFSTYNMHWVRQAPGQGLEWMGAIYPGNGETSYNQKFKDRVTLTVDKSTSTAYMELSSLRSEDTAVYYCAREDDYGEGYFDVWGQGTTVTVSS

SEQ ID NO: 64 - protein, 120 aa, synthetic construct
  REGION 1..120: note = Synthetic Sequence_hu119VH3
EVQLVQSGAEVKKPGSSVKVSCKASGFTFSTYNMHWVKHAPGQGLEWIGAIYPGNGETSYNQKFKDRATLTVDKSTSTAYMELSSLRSEDTAVYYCAREDDYGEGYFDVWGQGTTVTVSS

SEQ ID NO: 65 - protein, 120 aa, synthetic construct; identical to SEQ ID NO: 64
  REGION 1..120: note = Synthetic Sequence_hu119VH4

SEQ ID NO: 66 - protein, 120 aa, synthetic construct
  REGION 1..120: note = Synthetic Sequence_hu119VH5
EVQLVQSGAEVKKPGSSVKVSCKASGGTFSTYNMHWVRQAPGQGLEWIGAIYPGNGETSYNQKFKDRATLTVDKSTSTAYMELSSLRSEDTAVYYCAREDDYGEGYFDVWGQGTTVTVSS

SEQ ID NO: 67 - protein, 120 aa, synthetic construct
  REGION 1..120: note = Synthetic Sequence_hu119VH6
EAQLVQSGAEVKKPGSSVKVSCKASGFTFSTYNMHWVRQAPGQGLEWIGAIYPGNGETSYNQKFKDRATLTVDKSTSTAYMELSSLRSEDTAVYYCAREDDYGEGYFDVWGQGTTVTVSS

SEQ ID NO: 68 - protein, 120 aa, synthetic construct
  REGION 1..120: note = Synthetic Sequence_hu119VH7
EVQLVQSGAEVKKPGSSVKVSCKASGGTFSTYNMHWVRQAPGQGLEWIGAIYPGNGETSYNQKFKDRATLTVDKSTRTAYMELSSLRSEDTAVYYCAREDDYGEGYFDVWGQGTTVTVSS

SEQ ID NO: 69 - protein, 120 aa, synthetic construct
  REGION 1..120: note = Synthetic Sequence_hu119VH8
EAQLVQSGAEVKKPGSSVKVSCKASGFTFSTYNMHWVRQAPGQGLEWIGAIYPGNGETSYNQKFKDRATLTVDKSTRTAYMELSSLRSEDTAVYYCAREDDYGEGYFDVWGQGTTVTVSS

SEQ ID NO: 70 - protein, 15 aa, synthetic construct
  REGION 1..15: note = Synthetic Sequence_hu119 LCDR1-N31S
RASESVDSSGLSFMH

SEQ ID NO: 71 - protein, 15 aa, synthetic construct
  REGION 1..15: note = Synthetic Sequence_hu119 LCDR1-N31Q
RASESVDQSGLSFMH

SEQ ID NO: 72 - protein, 111 aa, synthetic construct
  REGION 1..111: note = Synthetic Sequence_hu119VL2-N31S
DIVLTQSPDSLAVSLGERATINCRASESVDSSGLSFMHWYQQKPGQPPKLLIYRASNLGSGVPDRFSGSGSGTDFTLTISSLQAEDVAVYYCQQINTDPLTFGQGTKLEIK

SEQ ID NO: 73 - protein, 111 aa, synthetic construct
  REGION 1..111: note = Synthetic Sequence_hu119VL2-N31Q
DIVLTQSPDSLAVSLGERATINCRASESVDQSGLSFMHWYQQKPGQPPKLLIYRASNLGSGVPDRFSGSGSGTDFTLTISSLQAEDVAVYYCQQINTDPLTFGQGTKLEIK

SEQ ID NO: 74 - protein, 111 aa, synthetic construct
  REGION 1..111: note = Synthetic Sequence_hu119VL6-N31S
DIVLTQSPATLSLSPGERATLSCRASESVDSSGLSFMHWYQQKPGQPPRLLLYRASNLGSGIPARFSGSGSGTDFTLTISSLEPEDFAVYYCQQINTDPLTFGQGTKLEIK

SEQ ID NO: 75 - protein, 111 aa, synthetic construct
  REGION 1..111: note = Synthetic Sequence_hu119VL6-N31Q
DIVLTQSPATLSLSPGERATLSCRASESVDQSGLSFMHWYQQKPGQPPRLLLYRASNLGSGIPARFSGSGSGTDFTLTISSLEPEDFAVYYCQQINTDPLTFGQGTKLEIK

SEQ ID NO: 76 - protein, 15 aa, synthetic construct
  REGION 1..15: note = Synthetic Sequence_hu119 LCDR1-general formula
  VARIANT 8: note = selected from N, S or Q
RASESVDXSGLSFMH

SEQ ID NO: 77 - protein, 107 aa, synthetic construct
  REGION 1..107: note = Synthetic Sequence_hu179VL1 (Graft (IGKV4-1*01))
DIVMTQSPDSLAVSLGERATINCKASQSVSSDVTWYQQKPGQPPKLLIYYVSNHYTGVPDRFSGSGSGTDFTLTISSLQAEDVAVYYCQQHHRFPLTFGQGTKLEIK

SEQ ID NO: 78 - protein, 107 aa, synthetic construct
  REGION 1..107: note = Synthetic Sequence_hu179VL2
DIVMTQSPDSLAVSLGERATINCKASQSVSSDVTWYQQKPGQSPKLLIYYVSNHYTGVPDRFSGSGSGTDFTLTISSLQAEDVAVYYCQQHHRFPLTFGQGTKLEIK

SEQ ID NO: 79 - protein, 107 aa, synthetic construct
  REGION 1..107: note = Synthetic Sequence_hu179 VL3
DIVMTQSPDSLAVSLGERATINCKASQSVSSDVTWYQQKPGQSPKLLIYYVSNHYTGVPDRFSGSGSGTDFTFTISSLQAEDVAVYYCQQHHRFPLTFGQGTKLEIK

SEQ ID NO: 80 - protein, 107 aa, synthetic construct
  REGION 1..107: note = Synthetic Sequence_hu179 VL4
SIVMTQSPDSLAVSLGERATINCKASQSVSSDVTWYQQKPGQSPKLLIYYVSNHYTGVPDRFSGSGSGTDFTLTISSLQAEDVAVYYCQQHHRFPLTFGQGTKLEIK

SEQ ID NO: 81 - protein, 107 aa, synthetic construct
  REGION 1..107: note = Synthetic Sequence_hu179VL5 (Grafted(IGKV2-29*02))
DIVMTQTPLSLSVTPGQPASISCKASQSVSSDVTWYLQKPGQSPQLLIYYVSNHYTGVPDRFSGSGSGTDFTLKISRVEAEDVGVYYCQQHHRFPLTFGQGTKLEIK

SEQ ID NO: 82 - protein, 107 aa, synthetic construct
  REGION 1..107: note = Synthetic Sequence_hu179VL6
SIVMTQTPLSLSVTPGQPASISCKASQSVSSDVTWYLQKPGQSPQLLIYYVSNHYTGVPDRFSGSGSGTDFTLKISRVEAEDVGVYYCQQHHRFPLTFGQGTKLEIK

SEQ ID NO: 83 - protein, 107 aa, synthetic construct
  REGION 1..107: note = Synthetic Sequence_hu179VL7
SIVMTQTPLSLSVTPGQPASISCKASQSVSSDVTWYLQKPGQSPQLLIYYVSNHYTGVPDRFSGSGSGTDFTFKISRVEAEDVGVYYCQQHHRFPLTFGQGTKLEIK

SEQ ID NO: 84 - protein, 107 aa, synthetic construct
  REGION 1..107: note = Synthetic Sequence_hu179VL8
SIVMTQTPLSLSVTPGQPASISCKASQSVSSDVTWYLQKPGQSPQLLIYYVSNHYTGVPDRFSGSGYGTDFTLKISRVEAEDVGVYYCQQHHRFPLTFGQGTKLEIK

SEQ ID NO: 85 - protein, 119 aa, synthetic construct
  REGION 1..119: note = Synthetic Sequence_hu179VH1 (Grafted)
EVQLVQSGAEVKKPGSSVKVSCKASGGTFSNYLIEWVRQAPGQGLEWMGVIDPGNGDTNYNENFKGRVTITADKSTSTAYMELSSLRSEDTAVYYCAREDNTGTAFDYWGQGTTVTVSS

SEQ ID NO: 86 - protein, 119 aa, synthetic construct
  REGION 1..119: note = Synthetic Sequence_hu179VH2
EVQLVQSGAEVKKPGSSVKVSCKASGYTFSNYLIEWVRQAPGQGLEWMGVIDPGNGDTNYNENFKGRVTLTADKSTSTAYMELSSLRSEDTAVYYCAREDNTGTAFDYWGQGTTVTVSS

SEQ ID NO: 87 - protein, 119 aa, synthetic construct
  REGION 1..119: note = Synthetic Sequence_hu179VH3
EVQLVQSGAEVKKPGSSVKVSCKASGYTFSNYLIEWVRQAPGQGLEWIGVIDPGNGDTNYNENFKGRATLTADKSTSTAYIELSSLRSEDTAVYYCAREDNTGTAFDYWGQGTTVTVSS

SEQ ID NO: 88 - protein, 119 aa, synthetic construct
  REGION 1..119: note = Synthetic Sequence_hu179VH4
EVQLVQSGAEVKKPGSSVKVSCKASGYTFSNYLIEWVKQAPGQGLEWIGVIDPGNGDTNYNENFKGKATLTADKSTSTAYIELSRLRSEDTAVYYCAREDNTGTAFDYWGQGTTVTVSS

SEQ ID NO: 89 - protein, 119 aa, synthetic construct
  REGION 1..119: note = Synthetic Sequence_hu179VH5
EVQLVQSGAEVKKPGSSVKVSCKASGYAFSNYLIEWVRQAPGQGLEWIGVIDPGNGDTNYNENFKGRATLTADKSTSTAYMELSSLRSEDTAVYYCAREDNTGTAFDYWGQGTTVTVSS

SEQ ID NO: 90 - protein, 119 aa, synthetic construct
  REGION 1..119: note = Synthetic Sequence_hu179VH1- N55Q
EVQLVQSGAEVKKPGSSVKVSCKASGGTFSNYLIEWVRQAPGQGLEWMGVIDPGQGDTNYNENFKGRVTITADKSTSTAYMELSSLRSEDTAVYYCAREDNTGTAFDYWGQGTTVTVSS

SEQ ID NO: 91 - protein, 119 aa, synthetic construct
  REGION 1..119: note = Synthetic Sequence_hu179VH1- N55V
EVQLVQSGAEVKKPGSSVKVSCKASGGTFSNYLIEWVRQAPGQGLEWMGVIDPGVGDTNYNENFKGRVTITADKSTSTAYMELSSLRSEDTAVYYCAREDNTGTAFDYWGQGTTVTVSS

SEQ ID NO: 92 - protein, 119 aa, synthetic construct
  REGION 1..119: note = Synthetic Sequence_hu179VH1- G56V
EVQLVQSGAEVKKPGSSVKVSCKASGGTFSNYLIEWVRQAPGQGLEWMGVIDPGNVDTNYNENFKGRVTITADKSTSTAYMELSSLRSEDTAVYYCAREDNTGTAFDYWGQGTTVTVSS

SEQ ID NO: 93 - protein, 17 aa, synthetic construct
  REGION 1..17: note = Synthetic Sequence_hu179 HCDR2-N55Q
VIDPGQGDTNYNENFKG

SEQ ID NO: 94 - protein, 17 aa, synthetic construct
  REGION 1..17: note = Synthetic Sequence_hu179 HCDR2-N55V
VIDPGVGDTNYNENFKG

SEQ ID NO: 95 - protein, 17 aa, synthetic construct
  REGION 1..17: note = Synthetic Sequence_hu179 HCDR2-G56V
VIDPGNVDTNYNENFKG

SEQ ID NO: 96 - protein, 17 aa, synthetic construct
  REGION 1..17: note = Synthetic Sequence_hu179 HCDR2 (general formula)
  VARIANT 6: note = selected from N, Q and V
  VARIANT 7: note = G or V
VIDPGXXDTNYNENFKG

SEQ ID NO: 97 - protein, 119 aa, synthetic construct
  REGION 1..119: note = Synthetic Sequence_hu179VH3-N55V
EVQLVQSGAEVKKPGSSVKVSCKASGYTFSNYLIEWVRQAPGQGLEWIGVIDPGVGDTNYNENFKGRATLTADKSTSTAYIELSSLRSEDTAVYYCAREDNTGTAFDYWGQGTTVTVSS

SEQ ID NO: 98 - protein, 107 aa, synthetic construct
  REGION 1..107: note = Synthetic Sequence_hu179VL2-Y50E
DIVMTQSPDSLAVSLGERATINCKASQSVSSDVTWYQQKPGQSPKLLIYEVSNHYTGVPDRFSGSGSGTDFTLTISSLQAEDVAVYYCQQHHRFPLTFGQGTKLEIK

SEQ ID NO: 99 - protein, 107 aa, synthetic construct
  REGION 1..107: note = Synthetic Sequence_hu179VL2-S52D
DIVMTQSPDSLAVSLGERATINCKASQSVSSDVTWYQQKPGQSPKLLIYYVDNHYTGVPDRFSGSGSGTDFTLTISSLQAEDVAVYYCQQHHRFPLTFGQGTKLEIK

SEQ ID NO: 100 - protein, 107 aa, synthetic construct
  REGION 1..107: note = Synthetic Sequence_hu179VL2-S52E
DIVMTQSPDSLAVSLGERATINCKASQSVSSDVTWYQQKPGQSPKLLIYYVENHYTGVPDRFSGSGSGTDFTLTISSLQAEDVAVYYCQQHHRFPLTFGQGTKLEIK

SEQ ID NO: 101 - protein, 107 aa, synthetic construct
  REGION 1..107: note = Synthetic Sequence_hu179VL2-N53Q
DIVMTQSPDSLAVSLGERATINCKASQSVSSDVTWYQQKPGQSPKLLIYYVSQHYTGVPDRFSGSGSGTDFTLTISSLQAEDVAVYYCQQHHRFPLTFGQGTKLEIK

SEQ ID NO: 102 - protein, 107 aa, synthetic construct
  REGION 1..107: note = Synthetic Sequence_hu179VL2-N53D
DIVMTQSPDSLAVSLGERATINCKASQSVSSDVTWYQQKPGQSPKLLIYYVSDHYTGVPDRFSGSGSGTDFTLTISSLQAEDVAVYYCQQHHRFPLTFGQGTKLEIK

SEQ ID NO: 103 - protein, 107 aa, synthetic construct
  REGION 1..107: note = Synthetic Sequence_hu179VL2-N53E
DIVMTQSPDSLAVSLGERATINCKASQSVSSDVTWYQQKPGQSPKLLIYYVSEHYTGVPDRFSGSGSGTDFTLTISSLQAEDVAVYYCQQHHRFPLTFGQGTKLEIK

SEQ ID NO: 104 - protein, 107 aa, synthetic construct
  REGION 1..107: note = Synthetic Sequence_hu179VL2-H54Y
DIVMTQSPDSLAVSLGERATINCKASQSVSSDVTWYQQKPGQSPKLLIYYVSNYYTGVPDRFSGSGSGTDFTLTISSLQAEDVAVYYCQQHHRFPLTFGQGTKLEIK

SEQ ID NO: 105 - protein, 107 aa, synthetic construct
  REGION 1..107: note = Synthetic Sequence_hu179VL2-H54D
DIVMTQSPDSLAVSLGERATINCKASQSVSSDVTWYQQKPGQSPKLLIYYVSNDYTGVPDRFSGSGSGTDFTLTISSLQAEDVAVYYCQQHHRFPLTFGQGTKLEIK

SEQ ID NO: 106 - protein, 107 aa, synthetic construct
  REGION 1..107: note = Synthetic Sequence_hu179VL2-H54E
DIVMTQSPDSLAVSLGERATINCKASQSVSSDVTWYQQKPGQSPKLLIYYVSNEYTGVPDRFSGSGSGTDFTLTISSLQAEDVAVYYCQQHHRFPLTFGQGTKLEIK

SEQ ID NO: 107 - protein, 107 aa, synthetic construct
  REGION 1..107: note = Synthetic Sequence_hu179VL2-Y55E
DIVMTQSPDSLAVSLGERATINCKASQSVSSDVTWYQQKPGQSPKLLIYYVSNHETGVPDRFSGSGSGTDFTLTISSLQAEDVAVYYCQQHHRFPLTFGQGTKLEIK

SEQ ID NO: 108 - protein, 7 aa, synthetic construct
  REGION 1..7: note = Synthetic Sequence_hu179 LCDR2-Y50E
EVSNHYT

SEQ ID NO: 109 - protein, 7 aa, synthetic construct
  REGION 1..7: note = Synthetic Sequence_hu179 LCDR2-S52D
YVDNHYT

SEQ ID NO: 110 - protein, 7 aa, synthetic construct
  REGION 1..7: note = Synthetic Sequence_hu179 LCDR2-S52E
YVENHYT

SEQ ID NO: 111 - protein, 7 aa, synthetic construct
  REGION 1..7: note = Synthetic Sequence_hu179LCDR2-N53Q
YVSQHYT

SEQ ID NO: 112 - protein, 7 aa, synthetic construct
  REGION 1..7: note = Synthetic Sequence_hu179 LCDR2-N53D
YVSDHYT

SEQ ID NO: 113 - protein, 7 aa, synthetic construct
  REGION 1..7: note = Synthetic Sequence_hu179 LCDR2-N53E
YVSEHYT

SEQ ID NO: 114 - protein, 7 aa, synthetic construct
  REGION 1..7: note = Synthetic Sequence_hu179 LCDR2-H54Y
YVSNYYT

SEQ ID NO: 115 - protein, 7 aa, synthetic construct
  REGION 1..7: note = Synthetic Sequence_hu179 LCDR2-H54D
YVSNDYT

SEQ ID NO: 116 - protein, 7 aa, synthetic construct
  REGION 1..7: note = Synthetic Sequence_hu179 LCDR2-H54E
YVSNEYT

SEQ ID NO: 117 - protein, 7 aa, synthetic construct
  REGION 1..7: note = Synthetic Sequence_hu179 LCDR2-Y55E
YVSNHET

SEQ ID NO: 118 - skipped

SEQ ID NO: 119 - protein, 107 aa, synthetic construct
  REGION 1..107: note = Synthetic Sequence_hu179VL8-N53E
SIVMTQTPLSLSVTPGQPASISCKASQSVSSDVTWYLQKPGQSPQLLIYYVSEHYTGVPDRFSGSGYGTDFTLKISRVEAEDVGVYYCQQHHRFPLTFGQGTKLEIK

SEQ ID NO: 120 - protein, 107 aa, synthetic construct
  REGION 1..107: note = Synthetic Sequence_hu199VL1 (Grafted)
DIQMTQSPSSLSASVGDRVTITCRASENIYSYLAWYQQKPGKAPKLLIYFAKTLAEGVPSRFSGSGSGTDFTLTISSLQPEDFATYYCQHHYGTPWTFGGGTKVEIK

SEQ ID NO: 121 - protein, 107 aa, synthetic construct
  REGION 1..107: note = Synthetic Sequence_hu199VL2
DIQMTQSPSSLSASVGDRVTITCRASENIYSYLAWYQQKPGKAPKLLVYFAKTLAEGVPSRFSGSGSGTDFTLTISSLQPEDFATYYCQHHYGTPWTFGGGTKVEIK

SEQ ID NO: 122 - protein, 107 aa, synthetic construct
  REGION 1..107: note = Synthetic Sequence_hu199VL3
DIQMTQSPSSLSASVGDRVTITCRASENIYSYLAWYQQKPGKSPQLLVYFAKTLAEGVPSRFSGSGSGTQFTLTISSLQPEDFATYYCQHHYGTPWTFGGGTKVEIK

SEQ ID NO: 123 - protein, 107 aa, synthetic construct
  REGION 1..107: note = Synthetic Sequence_hu199VL4
DIQMTQSPSSLSASVGDRVTITCRASENIYSYLAWYQQKPGKAPKLLIYFAKTLAEGVPSRFSGSVSGTDFTLTISSLQPEDFATYYCQHHYGTPWTFGGGTKVEIK

SEQ ID NO: 124 - protein, 107 aa, synthetic construct
  REGION 1..107: note = Synthetic Sequence_hu199VL5
DIQMTQSPSSLSASVGDRVTITCRASENIYSYLAWYQQKPGKAPKLLVYFAKTLAEGVPSRFSGSVSGTDFTLTISSLQPEDFATYYCQHHYGTPWTFGGGTKVEIK

SEQ ID NO: 125 - protein, 107 aa, synthetic construct
  REGION 1..107: note = Synthetic Sequence_hu199VL6
DIQMTQSPSSLSASVGDRVTITCRASENIYSYLAWYQQKPGKSPQLLVYFAKTLAEGVPSRFSGSVSGTQFTLTISSLQPEDFATYYCQHHYGTPWTFGGGTKVEIK

SEQ ID NO: 126 - protein, 117 aa, synthetic construct
  REGION 1..117: note = Synthetic Sequence_hu199VH1 (Grafted)
EVQLVQSGAEVKKPGASVKVSCKASGYTFTTYWMHWVRQAPGQGLEWMGMIDPSDSETTLIQKFKDRVTMTRDTSTSTVYMELSSLRSEDTAVYYCARTLDGYYDYWGQGTTVTVSS

SEQ ID NO: 127 - protein, 117 aa, synthetic construct
  REGION 1..117: note = Synthetic Sequence_hu199VH2
EVQLVQSGAEVKKPGASVKVSCKASGYTFTTYWMHWVRQAPGQGLEWMGMIDPSDSETTLIQKFKDRVTMTVDKSTSTAYMELSSLRSEDTAVYYCARTLDGYYDYWGQGTTVTVSS

SEQ ID NO: 128 - protein, 117 aa, synthetic construct
  REGION 1..117: note = Synthetic Sequence_hu199VH3
EVQLVQSGAEVKKPGASVKVSCKASGYTFTTYWMHWVRQAPGQGLEWMGMIDPSDSETTLIQKFKDRVTLTVDKSTSTAYMELSSLRSEDTAVYYCARTLDGYYDYWGQGTTVTVSS

SEQ ID NO: 129 - protein, 117 aa, synthetic construct
  REGION 1..117: note = Synthetic Sequence_hu199VH4
EVQLVQSGAEVKKPGASVKVSCKASGYTFTTYWMHWVRQAPGQGLEWIGMIDPSDSETTLIQKFKDRATLTVDKSTSTAYMELSSLRSEDTAVYYCARTLDGYYDYWGQGTTVTVSS

SEQ ID NO: 130 - protein, 117 aa, synthetic construct
  REGION 1..117: note = Synthetic Sequence_hu199VH5
EVQLVQSGAEVKKPGASVKVSCKASGYTFTTYWMHWVKQAPGQGLEWIGMIDPSDSETTLIQKFKDKATLTVDKSTSTAYMELSSLRSEDTAVYYCARTLDGYYDYWGQGTTVTVSS

SEQ ID NO: 131 - protein, 117 aa, synthetic construct
  REGION 1..117: note = Synthetic Sequence_hu199VH6
EVQLVQSGAEVKKPGASVKVSCKASGYTFTTYWMHWVKQAPGQGLEWMGMIDPSDSETTLIQKFKDKVTMTVDKSTSTAYMELSSLRSEDTAVYYCARTLDGYYDYWGQGTTVTVSS

SEQ ID NO: 132 - protein, 117 aa, synthetic construct
  REGION 1..117: note = Synthetic Sequence_hu199VH7
EVQLVQSGAEVKKPGASVKVSCKASGYTFTTYWMHWVKQAPGQGLEWMGMIDPSDSETTLIQKFKDKVTLTVDKSTSTAYMELSSLRSEDTAVYYCARTLDGYYDYWGQGTTVTVSS

SEQ ID NO: 133 - protein, 330 aa, synthetic construct
  REGION 1..330: note = Synthetic Sequence_IgG1-YTE heavy chain constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 107 aa, synthetic construct [sequence omitted]

SEQ ID NO: 135 - protein, 451 aa, synthetic construct
  REGION 1..451: note = Synthetic Sequence_hu3-13 antibody heavy chain
EVQLVQSGAEVKKPGASVKVSCKASGYTFTDDYMNWVRQAPGQRLEWMGIISPYNGGTSYNQKFKGRVTLTVDKSASTAYMELSSLRSEDTAVYYCAREDYDYDGYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 212 aa, synthetic construct
  REGION 1..212: note = Synthetic Sequence_hu3-13 antibody light chain
EIVLTQSPATLSLSPGERATLSCRASSSVSYMHWYQQKPGQAPRPWIYATSNLASGVPARFSGSGSGTSYTLTISRLEPEDFAVYYCQQSDSGREFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 450 aa, synthetic construct
  REGION 1..450: note = Synthetic Sequence_hu119-30 antibody heavy chain
EAQLVQSGAEVKKPGSSVKVSCKASGFTFSTYNMHWVRQAPGQGLEWIGAIYPGNGETSYNQKFKDRATLTVDKSTRTAYMELSSLRSEDTAVYYCAREDDYGEGYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 218 aa, synthetic construct
  REGION 1..218: note = Synthetic Sequence_hu119-30 antibody light chain
DIVLTQSPATLSLSPGERATLSCRASESVDSSGLSFMHWYQQKPGQPPRLLLYRASNLGSGIPARFSGSGSGTDFTLTISSLEPEDFAVYYCQQINTDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 449 aa, synthetic construct
  REGION 1..449: note = Synthetic Sequence_hu179-33 antibody heavy chain
EVQLVQSGAEVKKPGSSVKVSCKASGYTFSNYLIEWVRQAPGQGLEWIGVIDPGVGDTNYNENFKGRATLTADKSTSTAYIELSSLRSEDTAVYYCAREDNTGTAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 214 aa, synthetic construct
  REGION 1..214: note = Synthetic Sequence_hu179-33 antibody light chain
SIVMTQTPLSLSVTPGQPASISCKASQSVSSDVTWYLQKPGQSPQLLIYYVSEHYTGVPDRFSGSGYGTDFTLKISRVEAEDVGVYYCQQHHRFPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 447 aa, synthetic construct
  REGION 1..447: note = Synthetic Sequence_hu199-36 antibody heavy chain
EVQLVQSGAEVKKPGASVKVSCKASGYTFTTYWMHWVKQAPGQGLEWMGMIDPSDSETTLIQKFKDKVTLTVDKSTSTAYMELSSLRSEDTAVYYCARTLDGYYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 214 aa, synthetic construct
  REGION 1..214: note = Synthetic Sequence_hu199-36 antibody light chain
DIQMTQSPSSLSASVGDRVTITCRASENIYSYLAWYQQKPGKSPQLLVYFAKTLAEGVPSRFSGSVSGTQFTLTISSLQPEDFATYYCQHHYGTPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 448 aa, synthetic construct
  REGION 1..448: note = Synthetic Sequence_AMG157 heavy chain sequence
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 144 - protein, 214 aa, synthetic construct
  REGION 1..214: note = Synthetic Sequence_AMG157 light chain sequence
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 145 - protein, 371 aa, synthetic construct
  REGION 1..371: note = Synthetic Sequence_Amino acid sequence of human TSLP receptor full-length sequence
MGRLVLLWGAAVFLLGGWMALGQGGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKFILISSLAILLMVSLLLLSLWKLWRVKKFLIPSVPDPKSIFPGLFEIHQGNFQEWITDTQNVAHLHKMAGAEQESGPEEPLVVQLAKTEAESPRMLDPQTEEKEASGGSLQLPHQPLQGGDVVTIGGFTFVMNDRSYVAL

SEQ ID NO: 146 - protein, 459 aa, synthetic construct [sequence omitted]

(19) United States
(12) Patent Application Publication
(10) Pub. No.: US 2026/0022166 A1
(43) Pub. Date: Jan. 22, 2026

(54) ANTIBODY CAPABLE OF BINDING TO THYMIC STROMAL LYMPHOPOIETIN AND USE THEROF

(71) Applicants: JIANGSU HENGRUI MEDICINE CO., LTD., Lianyungang (CN); SHANGHAI HENGRUI PHARMACEUTICAL CO., LTD., Shanghai (CN)

(72) Inventors: Jinping SHI, Shanghai (CN); Hua YING, Shanghai (CN); Tingting LI, Shanghai (CN); Yifang WANG, Shanghai (CN); Guimei YANG, Shanghai (CN); Hu GE, Shanghai (CN); Weikang TAO, Shanghai (CN)

(21) Appl. No.: 19/284,844

(22) Filed: Jul. 30, 2025

Related U.S. Application Data
(63) Continuation of application No. 17/615,970, now Pat. No. 12,404,323.

(30) Foreign Application Priority Data
Jun. 4, 2019 (CN) 201910480579.9

(51) Int. Cl. C07K 16/24; A61K 39/00; A61P 11/06

(52) U.S. Cl. CPC C07K 16/24; A61P 11/06; A61K 2039/505; C07K 2317/24; C07K 2317/565; C07K 2317/76; C07K 2317/92

(57) ABSTRACT

Disclosed are an antibody capable of binding to thymic stromal lymphopoietin and the use thereof. Disclosed are an anti-TSLP antibody, comprising a murine antibody, chimeric antibody and humanized antibody of the light chain and heavy chain variable regions of the anti-TSLP antibody and antigen-binding fragments thereof, or a pharmaceutically acceptable salt or solvent compound thereof, and the use thereof as a medicament for treating asthma, especially the use thereof in the preparation of a drug for treating TSLP-positive diseases or conditions.

### CROSS REFERENCE TO RELATED APPLICATIONS

The present application is a continuation patent application which claims the benefit of and priority to U.S. patent application Ser. No. 17/615,970, filed on Dec. 2, 2021, which is the national stage of International Patent Application No. PCT/CN2020/094154, filed on Jun. 3, 2020, which claims the benefit of and priority to Chinese Patent Application No. 201910480579.9 filed on Jun. 4, 2019, which are incorporated herein by reference in their entirety.

### SEQUENCE LISTING

The instant application contains a Sequence Listing which has been submitted electronically in XML format and is hereby incorporated by reference in its entirety. Said XML copy, created on Sep. 3, 2025, is named “AIO001US03_ST26_REPLACEMENT.xml” and is 188,761 bytes in size.

### FILED OF INVENTION

The present disclosure relates to the field of antibody agents. Specifically, the present disclosure relates to anti-TSLP antibody agents and the use thereof.

### BACKGROUND OF THE INVENTION

The statements herein only provide background information related to the present disclosure and do not necessarily constitute the prior art.

Asthma is a serious chronic inflammatory airway disease. There are about 334 million asthma patients worldwide and about 30 million asthma patients in China, where the mortality rate is much higher than that in developed countries. As the environment deteriorates and air pollution increases, more people may suffer from this disease, which will seriously endanger human life and health.

Thymic stromal lymphopoietin (TSLP) is an epithelial cell-derived cytokine produced in response to pro-inflammatory stimuli. It mainly promotes allergic inflammation through its activity on dendritic cells and mast cells. TSLP is a type of interleukin 7 (IL-7)-like cytokine, which was first discovered in the conditioned medium of mouse thymic stromal cells. TSLP is mainly expressed in lung, skin and intestinal epithelial cells. TSLP consists of 4 α-helices and two loops AB and CD. In the molecule, there are three pairs of disulfide bonds consisting of six cysteine, two N-glycosylation sites, and the molecular weight is about 15-20 kD. TSLP receptor is a complex consisting of two moieties, one is TSLPR and the other is IL7Rα. TSLP first binds to TSLPR with relatively low affinity, then recruits the binding of IL7Rα with high affinity, and finally activates signal pathways of stat5, etc., leading to the maturation of DCs and the differentiation of T cells.

Myeloid dendritic cells (mDCs) are the major effector cells for TSLP. TSLP acts on immature mDCs, which secrete cytokines IL-8, eotaxin-2, TARC and MDC, while highly express OX40L. In the absence of IL-12, OX40L binds to the native CD4+ T cells, leading to their differentiation into Th2 cells. Th2 cells then secrete Th2 cytokines such as IL-5, IL-4, IL-9, IL-9 and TNF, inducing Th2 inflammatory response in the body. In addition, TSLP can also induce DC cells to produce the cytokine IL-8, which recruits neutrophils in turn, leading to neutrophilic innate immune inflammation. TSLP can also induce DCs to produce eotaxin-2, which recruits eosinophils, and acts together with IL5 to make the body quickly enter the inflammatory state of eosinophil infiltration. TSLP also acts on mast cells and natural killer cells, and mediates innate inflammation by inducing the production of IL-4, IL-6, IgE, etc. In summary, TSLP can cause innate inflammation and Th2 inflammation at the same time, which in turn increases tissue mucus, remodels the airway, which leads to tracheal stenosis, and makes cell fibrosis become severe. The inflammation gradually evolves into the three major allergic diseases, asthma, allergic dermatitis and allergic rhinitis. Therefore, blocking TSLP is a potentially effective strategy for the treatment of diseases such as asthma, allergic dermatitis, etc.

Currently, anti-TSLP antibodies are disclosed in WO2008155365, WO2009035577, WO2011056772, WO2016142426 and WO2017004149. However, there is no corresponding antibody commercially available. Therefore, it is necessary to continue the development of effective medicament for treating TSLP-related diseases.

### SUMMARY OF THE INVENTION

The present disclosure provides an anti-TSLP antibody.

In some embodiments, the anti-TSLP antibody as described above comprises an antibody heavy chain variable region and a light chain variable region, wherein:
i) the heavy chain variable region comprises HCDR1, HCDR2 and HCDR3 as shown in SEQ ID NO: 14, SEQ ID NO: 15 and SEQ ID NO: 47, respectively, and the light chain variable region comprises LCDR1, LCDR2 as shown in SEQ ID NO: 17, SEQ ID NO: 18, respectively, and LCDR3 as shown in SEQ ID NO: 48 or 55;
wherein, the sequence of SEQ ID NO: 47 is EDYDYDGYAMDX₁, the sequence of SEQ ID NO: 48 is QQWSSX₂RT, the sequence of SEQ ID NO: 55 is QQSDX₃X₄RX₅, wherein X₁ is H or Y, X₂ is N or D, X₃ is N or S, X₄ is V or G, X₅ is G or E; or
ii) the heavy chain variable region comprises HCDR1, HCDR2 and HCDR3 as shown in SEQ ID NO: 20, SEQ ID NO: 21 and SEQ ID NO: 22, respectively, and the light chain variable region comprises LCDR1, LCDR2 and LCDR3 as shown in SEQ ID NO: 76, SEQ ID NO: 24 and SEQ ID NO: 25, respectively;
wherein, the sequence of SEQ ID NO: 76 is RASESVDX₆SGLSFMH, wherein, X₆ is selected from N, S or Q; or
iii) the heavy chain variable region comprises HCDR1, HCDR2 and HCDR3 as shown in SEQ ID NO: 26, SEQ ID NO: 96 and SEQ ID NO: 28, respectively, and the light chain variable region comprises LCDR1, LCDR2 and LCDR3 as shown in SEQ ID NO: 29, SEQ ID NO: 118 and SEQ ID NO: 31, respectively;
wherein, the sequence of SEQ ID NO: 96 is VIDPGX₇X₈DTNYNE, the sequence of SEQ ID NO: 118 is X₉VX₁₀X₁₁X₁₂X₁₃T, wherein X₇ is selected from N, Q and V, X₈ is G or V; X₉ is Y or E, X₁₀ is selected from S, D and E, X₁₁ is selected from N, Q, D and E, X₁₂ is selected from H, Y, D and E, X₁₃ is E or Y; or
iv) the heavy chain variable region comprises HCDR1, HCDR2 and HCDR3 as shown in SEQ ID NO: 32, SEQ ID NO: 33 and SEQ ID NO: 34, respectively, and the light chain variable region comprises LCDR1, LCDR2 and LCDR3 as shown in SEQ ID NO: 35, SEQ ID NO: 36 and SEQ ID NO: 37, respectively.

In some embodiments, the anti-TSLP antibody as described above comprises a heavy chain variable region and a light chain variable region, wherein:
i) the heavy chain variable region comprises HCDR1, HCDR2 and HCDR3 as shown in SEQ ID NO: 14, SEQ ID NO: 15 and SEQ ID NO: 16, respectively, and the light chain variable region comprises LCDR1, LCDR2 and LCDR3 as shown in SEQ ID NO: 17, SEQ ID NO: 18 and SEQ ID NO: 19, respectively; or
ii) the heavy chain variable region comprises HCDR1, HCDR2 and HCDR3 as shown in SEQ ID NO: 14, SEQ ID NO: 15 and SEQ ID NO: 45, respectively, and the light chain variable region comprises LCDR1, LCDR2 and LCDR3 as shown in SEQ ID NO: 17, SEQ ID NO: 18 and SEQ ID NO: 46, respectively; or
iii) the heavy chain variable region comprises HCDR1, HCDR2 and HCDR3 as shown in SEQ ID NO: 14, SEQ ID NO: 15 and SEQ ID NO: 45, respectively, and the light chain variable region comprises LCDR1, LCDR2 and LCDR3 as shown in SEQ ID NO: 17, SEQ ID NO: 18 and SEQ ID NO: 53, respectively; or
iv) the heavy chain variable region comprises HCDR1, HCDR2 and HCDR3 as shown in SEQ ID NO: 14, SEQ ID NO: 15 and SEQ ID NO: 45, respectively, and the light chain variable region comprises LCDR1, LCDR2 and LCDR3 as shown in SEQ ID NO: 17, SEQ ID NO: 18 and SEQ ID NO: 54, respectively; or
v) the heavy chain variable region comprises HCDR1, HCDR2 and HCDR3 as shown in SEQ ID NO: 20, SEQ ID NO: 21 and SEQ ID NO: 22, respectively, and the light chain variable region comprises LCDR1, LCDR2 and LCDR3 as shown in SEQ ID NO: 23, SEQ ID NO: 24 and SEQ ID NO: 25, respectively; or
vi) the heavy chain variable region comprises HCDR1, HCDR2 and HCDR3 as shown in SEQ ID NO: 20, SEQ ID NO: 21 and SEQ ID NO: 22, respectively, and the light chain variable region comprises LCDR1, LCDR2 and LCDR3 as shown in SEQ ID NO: 70, SEQ ID NO: 24 and SEQ ID NO: 25, respectively; or
vii) the heavy chain variable region comprises HCDR1, HCDR2 and HCDR3 as shown in SEQ ID NO: 20, SEQ ID NO: 21 and SEQ ID NO: 22, respectively, and the light chain variable region comprises LCDR1, LCDR2 and LCDR3 as shown in SEQ ID NO: 71, SEQ ID NO: 24 and SEQ ID NO: 25, respectively; or
viii) the heavy chain variable region comprises HCDR1 and HCDR3 as shown in SEQ ID NO: 26, SEQ ID NO: 28, respectively, and HCDR2 as shown in SEQ ID NO: 27, 93, 94 or 95, and the light chain variable region comprises LCDR1 and LCDR3 as shown in SEQ ID NO: 29, SEQ ID NO: 31, respectively, and LCDR2 as shown in SEQ ID NO: 30, 108, 109, 110, 111, 112, 113, 114, 115, 116 or 117.

In some embodiments, the anti-TSLP antibody as described above comprises a heavy chain variable region and a light chain variable region, wherein:
a) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 30 and SEQ ID NO: 31, respectively; or
b) the heavy chain variable region comprises HCDR1, HCDR2 and HCDR3 as shown in SEQ ID NO: 26, SEQ ID NO: 93 and SEQ ID NO: 28, respectively, and the light chain variable region comprises LCDR1, LCDR2 and LCDR3 as shown in SEQ ID NO: 29, SEQ ID NO: 30 and SEQ ID NO: 31, respectively; or
c) the heavy chain variable region comprises HCDR1, HCDR2 and HCDR3 as shown in SEQ ID NO: 26, SEQ ID NO: 94 and SEQ ID NO: 28, respectively, and the light chain variable region comprises LCDR1, LCDR2 and LCDR3 as shown in SEQ ID NO: 29, SEQ ID NO: 30 and SEQ ID NO: 31, respectively; or
d) the heavy chain variable region comprises HCDR1, HCDR2 and HCDR3 as shown in SEQ ID NO: 26, SEQ ID NO: 95 and SEQ ID NO: 28, respectively, and the light chain variable region comprises LCDR1, LCDR2 and LCDR3 as shown in SEQ ID NO: 29, SEQ ID NO: 30 and SEQ ID NO: 31, respectively; or
e) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 108 and SEQ ID NO: 31, respectively; or
f) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 109 and SEQ ID NO: 31, respectively; or
g) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 110 and SEQ ID NO: 31, respectively; or
h) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 111 and SEQ ID NO: 31, respectively; or
i) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 112 and SEQ ID NO: 31, respectively; or
j) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 113 and SEQ ID NO: 31, respectively; or
k) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 114 and SEQ ID NO: 31, respectively; or
l) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 115 and SEQ ID NO: 31, respectively; or
m) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 116 and SEQ ID NO: 31, respectively; or
n) the heavy chain variable region comprises HCDR1, HCDR2 and HCDR3 as shown in SEQ ID NO: 26, SEQ ID NO: 27 and SEQ ID NO: 28, respectively, and the light chain variable region comprises LCDR1, LCDR2 and LCDR3 as shown in SEQ ID NO: 29, SEQ ID NO: 117 and SEQ ID NO: 31, respectively.

In some embodiments of the anti-TSLP antibody as described above, the anti-TSLP antibody is a murine antibody, a chimeric antibody or a humanized antibody.

In some embodiments of the anti-TSLP antibody as described above, the anti-TSLP antibody comprises framework region(s) derived from a human antibody, or the anti-TSLP antibody comprises a light chain variable region and/or a heavy chain variable region selected from those described in (a), (b), (c) or (d) below:
a) the heavy chain variable region comprises HCDR1 and HCDR2 as shown in SEQ ID NO: 14, SEQ ID NO: 15, respectively, and HCDR3 as shown in SEQ ID NO: 16 or 45, and the framework region(s) thereof comprise(s) at most 10 back mutations, preferably, the back mutation is selected from one or more of 38K, 48I, 67A, 69L, 71V and 73K; and/or the light chain variable region comprises LCDR1 and LCDR2 as shown in SEQ ID NO: 17, SEQ ID NO: 18, respectively, and LCDR3 as shown in SEQ ID NO: 19, 46, 53 or 54, and the framework region(s) thereof comprise(s) at most 10 amino acid back mutations, preferably, the back mutation is selected from one or more of 46P, 47W, 58V, 70S and 71Y;
b) the heavy chain variable region comprises HCDR1, HCDR2 and HCDR3 as shown in SEQ ID NO: 20, SEQ ID NO: 21 and SEQ ID NO: 22, respectively, and the framework region(s) thereof comprise(s) at most 10 back mutations, preferably, the back mutation is selected from one or more of 2A, 27F, 38K, 39H, 48I, 67A, 69L, 71V and 76R; and/or the light chain variable region comprises LCDR2 and LCDR3 as shown in SEQ ID NO: 24, SEQ ID NO: 25, respectively, and LCDR1 as shown in SEQ ID NO: 23, 70 or 71, and the framework region(s) thereof comprise(s) at most 10 amino acid back mutations, preferably, the back mutation is one or more of 1D, 4L, 43P, 48L and 58I;
c) the heavy chain variable region comprises HCDR1 and HCDR3 as shown in SEQ ID NO: 26, SEQ ID NO: 28, respectively, and HCDR2 as shown in SEQ ID NO: 27, 93, 94 or 95, and the framework region(s) thereof comprise(s) at most 10 back mutations, preferably, the back mutation is selected from one or more of 27Y, 28A, 38K, 48I, 66K, 67A, 69L, 80I and 82b R; and/or the light chain variable region comprises LCDR1 and LCDR3 as shown in SEQ ID NO: 29, SEQ ID NO: 31, respectively, and LCDR2 as shown in SEQ ID NO: 30, 108, 109, 110, 111, 112, 113, 114, 115, 116 or 117, and the framework region(s) thereof comprise(s) at most 10 back mutations, preferably, the back mutation is selected from one or more of 1S, 43S, 67Y and 73F; or
d) the heavy chain variable region comprises HCDR1, HCDR2 and HCDR3 as shown in SEQ ID NO: 32, SEQ ID NO: 33 and SEQ ID NO: 34, respectively, and the framework region(s) thereof comprise(s) at most 10 back mutations, preferably, the back mutation is selected from one or more of 38K, 48I, 66K, 67A, 69L, 71V, 73K and 78A; and/or the light chain variable region comprises LCDR1, LCDR2 and LCDR3 as shown in SEQ ID NO: 35, SEQ ID NO: 36 and SEQ ID NO: 37, respectively, and the framework region(s) thereof comprise(s) at most 10 back mutations, preferably, the back mutation is selected from one or more of 43S, 45Q, 48V, 66V and 70Q.

In some embodiments, the anti-TSLP antibody as described above comprises a heavy chain variable region and a light chain variable region, wherein:
i) the heavy chain variable region has at least 90%, 92%, 94%, 95%, 96%, 97%, 98% or 99% sequence identity with the heavy chain variable region as shown in amino acid sequence SEQ ID NO: 6, 42, 43, 44 or 50, and the light chain variable region has at least 90%, 92%, 94%, 95%, 96%, 97%, 98% or 99% sequence identity with the light chain variable region as shown in amino acid sequence SEQ ID NO: 7, 38, 39, 40, 41, 49, 51 or 52; or
ii) the heavy chain variable region has at least 90%, 92%, 94%, 95%, 96%, 97%, 98% or 99% sequence identity with the heavy chain variable region as shown in amino acid sequence SEQ ID NO: 8, 62, 63, 64, 65, 66, 67, 68 or 69, and the light chain variable region has at least 90%, 92%, 94%, 95%, 96%, 97%, 98% or 99% sequence identity with the light chain variable region as shown in amino acid sequence SEQ ID NO: 9, 56, 57, 58, 59, 60, 61, 72, 73, 74 or 75; or
iii) the heavy chain variable region has at least 90%, 92%, 94%, 95%, 96%, 97%, 98% or 99% sequence identity with the heavy chain variable region as shown in amino acid sequence SEQ ID NO: 10, 85, 86, 87, 88, 89, 90, 91, 92 or 97, and the light chain variable region has at least 90%, 92%, 94%, 95%, 96%, 97%, 98% or 99% sequence identity with the light chain variable region as shown in amino acid sequence SEQ ID NO: 11, 77, 78, 79, 80, 81, 82, 83, 84, 98, 99, 100, 101, 102, 103, 104, 105, 106, 107 or 119; or
iv) the heavy chain variable region has at least 90%, 92%, 94%, 95%, 96%, 97%, 98% or 99% sequence identity with the heavy chain variable region as shown in amino acid sequence SEQ ID NO: 12, 126, 127, 128, 129, 130, 131 or 132, and the light chain variable region has at least 90%, 92%, 94%, 95%, 96%, 97%, 98% or 99% sequence identity with the light chain variable region as shown in amino acid sequence SEQ ID NO: 13, 120, 121, 122, 123, 124 or 125.

In some embodiments of the anti-TSLP antibody as described above, the anti-TSLP antibody is a humanized antibody, which comprises framework region(s) derived from a human antibody or a framework region variant thereof, said framework region variant has at most 1, 2, 3, 4, 5, 6, 7, 8, 9 or 10 amino acid back mutations in the light chain framework region(s) and/or heavy chain framework region(s) of the human antibody, respectively.

In some embodiments of the anti-TSLP antibody as described above, the framework region variant comprises back mutations selected from those described in (a), (b), (c) or (d) below:
a) one or more amino acid back mutations selected from the group consisting of 46P, 47W, 58V, 70S and 71Y comprised in the framework region(s) of the light chain variable region as shown in SEQ ID NO: 38, 49, 51 or 52, and/or one or more amino acid back mutations selected from the group consisting of 38K, 48I, 67A, 69L, 71V and 73K comprised in the framework region(s) of the heavy chain variable region as shown in SEQ ID NO: 42 or 50;
b) one or more amino acid back mutations selected from the group consisting of 1D, 4L, 43P, 48L and 58I comprised in the framework region(s) of the light chain variable region as shown in SEQ ID NO: 56, 59, 72, 73, 74 or 75, and/or one or more amino acid back mutations selected from the group consisting of 2A, 27F, 38K, 39H, 48I, 67A, 69L, 71V and 76R comprised in the framework region(s) of the heavy chain variable region as shown in SEQ ID NO: 62;
c) one or more amino acid back mutations selected from the group consisting of 1S, 43 S, 67Y and 73F comprised in the framework region(s) of the light chain variable region as shown in SEQ ID NO: 77, 81, 98, 99, 100, 101, 102, 103, 104, 105, 106, 107 or 119, and/or one or more amino acid back mutations selected from the group consisting of 27Y, 28A, 38K, 48I, 66K, 67A, 69L, 80I and 82b R comprised in the framework region(s) of the heavy chain variable region as shown in SEQ ID NO: 85, 90, 91, 92 or 97;
d) one or more amino acid back mutations selected from the group consisting of 43S, 45Q, 48V, 66V and 70Q comprised in the framework region(s) of the light chain variable region as shown in SEQ ID NO: 120, and/or one or more amino acid back mutations selected from the group consisting of 38K, 48I, 66K, 67A, 69L, 71V, 73K and 78A comprised in the framework region of the heavy chain variable region as shown in SEQ ID NO: 126.

In some embodiments, the anti-TSLP antibody as described above comprises a heavy chain variable region and a light chain variable region, wherein:
i) the amino acid sequence of the heavy chain variable region is as shown in SEQ ID NO: 6, 42, 43, 44 or 50, and the amino acid sequence of the light chain variable region is as shown in SEQ ID NO: 7, 38, 39, 40, 41, 49, 51 or 52; or
ii) the amino acid sequence of the heavy chain variable region is as shown in SEQ ID NO: 8, 62, 63, 64, 65, 66, 67, 68 or 69, and the amino acid sequence of the light chain variable region is as shown in SEQ ID NO: 9, 56, 57, 58, 59, 60, 61, 72, 73, 74 or 75; or
iii) the amino acid sequence of the heavy chain variable region is as shown in SEQ ID NO: 10, 85, 86, 87, 88, 89, 90, 91, 92 or 97, and the amino acid sequence of the light chain variable region is as shown in SEQ ID NO: 11, 77, 78, 79, 80, 81, 82, 83, 84, 98, 99, 100, 101, 102, 103, 104, 105, 106, 107 or 119; or
iv) the amino acid sequence of the heavy chain variable region is as shown in SEQ ID NO: 12, 126, 127, 128, 129, 130, 131 or 132, and the amino acid sequence of the light chain variable region is as shown in SEQ ID NO: 13, 120, 121, 122, 123, 124 or 125.

In some embodiments, the anti-TSLP antibody as described above comprises a heavy chain variable region and a light chain variable region as shown below:
(a) the sequence of the heavy chain variable region is as shown in SEQ ID NO: 6, and the sequence of the light chain variable region is as shown in SEQ ID NO: 7;
(b) the sequence of the heavy chain variable region is as shown in SEQ ID NO: 42, 43 or 44, and the sequence of the light chain variable region is as shown in SEQ ID NO: 39, 40 or 41;
(c) the sequence of the heavy chain variable region is as shown in SEQ ID NO: 43, and the sequence of the light chain variable region is as shown in SEQ ID NO: 38;
(d) the sequence of the heavy chain variable region is as shown in SEQ ID NO: 50, and the sequence of the light chain variable region is as shown in SEQ ID NO: 49, 51 or 52;
(e) the sequence of the heavy chain variable region is as shown in SEQ ID NO: 8, and the sequence of the light chain variable region is as shown in SEQ ID NO: 9;
(f) the sequence of the heavy chain variable region is as shown in SEQ ID NO: 62, 63, 64 or 65, and the sequence of the light chain variable region is as shown in SEQ ID NO: 56, 57 or 58;
(g) the sequence of the heavy chain variable region is as shown in SEQ ID NO: 64, 66, 67, 68 or 69, and the sequence of the light chain variable region is as shown in SEQ ID NO: 59, 60 or 61;
(h) the sequence of the heavy chain variable region is as shown in SEQ ID NO: 64, and the sequence of the light chain variable region is as shown in SEQ ID NO: 72 or 73;
(i) the sequence of the heavy chain variable region is as shown in SEQ ID NO: 69, and the sequence of the light chain variable region is as shown in SEQ ID NO: 74;
(j) the sequence of the heavy chain variable region is as shown in SEQ ID NO: 10, and the sequence of the light chain variable region is as shown in SEQ ID NO: 11;
(k) the sequence of the heavy chain variable region is as shown in SEQ ID NO: 85, and the sequence of the light chain variable region is as shown in SEQ ID NO: 77, 78, 102 or 104;
(l) the sequence of the heavy chain variable region is as shown in SEQ ID NO: 86 or 88, and the sequence of the light chain variable region is as shown in SEQ ID NO: 77 or 78;
(m) the sequence of the heavy chain variable region is as shown in SEQ ID NO: 87, and the sequence of the light chain variable region is as shown in SEQ ID NO: 77, 78, 79, 81, 82, 83, 84, 98, 99, 100, 101, 103, 105, 106 or 107;
(n) the sequence of the heavy chain variable region is as shown in SEQ ID NO: 89, and the sequence of the light chain variable region is as shown in SEQ ID NO: 79, 81, 82, 83 or 84;
(o) the sequence of the heavy chain variable region is as shown in SEQ ID NO: 90, 91 or 92, and the sequence of the light chain variable region is as shown in SEQ ID NO: 78;
(p) the sequence of the heavy chain variable region is as shown in SEQ ID NO: 97, and the sequence of the light chain variable region is as shown in SEQ ID NO: 119;
(q) the sequence of the heavy chain variable region is as shown in SEQ ID NO: 12, and the sequence of the light chain variable region is as shown in SEQ ID NO: 13;
(r) the sequence of the heavy chain variable region is as shown in SEQ ID NO: 127, 128, 129, 130, 131 or 132, and the sequence of the light chain variable region is as shown in SEQ ID NO: 120, 121, 123, 124 or 125; or
(s) the sequence of the heavy chain variable region is as shown in SEQ ID NO: 132, and the sequence of the light chain variable region is as shown in SEQ ID NO: 125.

In some embodiments of the anti-TSLP antibody as described above, the combinations of the light chain variable region and the heavy chain variable region of the antibodies are shown as follows:

TABLE 1
Combinations of the light and heavy chain variable
regions of the mAb3 humanized antibodies

| | VH (SEQ | VL (SEQ |
|---|---|---|
| Antibody | ID NO) | ID NO) |
| hu3-01 | 42 | 39 |
| hu3-02 | 42 | 40 |
| hu3-03 | 42 | 41 |
| hu3-04 | 43 | 38 |
| hu3-05 | 43 | 39 |
| hu3-06 | 43 | 40 |
| hu3-07 | 43 | 41 |
| hu3-08 | 44 | 39 |
| hu3-09 | 44 | 40 |
| hu3-10 | 44 | 41 |
| hu3-11 | 50 | 49 |
| hu3-12 | 50 | 51 |
| hu3-13 | 50 | 52 |

TABLE 2
Combinations of the light and heavy chain variable
regions of the mAb119 humanized antibodies

| | VH (SEQ | VL (SEQ |
|---|---|---|
| Antibody | ID NO) | ID NO) |
| hu119-01 | 62 | 56 |
| hu119-02 | 63 | 56 |
| hu119-03 | 64 | 56 |
| hu119-04 | 65 | 56 |
| hu119-05 | 62 | 57 |
| hu119-06 | 63 | 57 |
| hu119-07 | 64 | 57 |
| hu119-08 | 65 | 57 |
| hu119-09 | 62 | 58 |
| hu119-10 | 63 | 58 |
| hu119-11 | 64 | 58 |
| hu119-12 | 65 | 58 |
| hu119-13 | 64 | 59 |
| hu119-14 | 66 | 59 |
| hu119-15 | 67 | 59 |
| hu119-16 | 68 | 59 |
| hu119-17 | 69 | 59 |
| hu119-18 | 64 | 60 |
| hu119-19 | 66 | 60 |
| hu119-20 | 67 | 60 |
| hu119-21 | 68 | 60 |
| hu119-22 | 69 | 60 |
| hu119-23 | 64 | 61 |
| hu119-24 | 66 | 61 |
| hu119-25 | 67 | 61 |
| hu119-26 | 68 | 61 |
| hu119-27 | 69 | 61 |
| hu119-28 | 64 | 72 |
| hu119-29 | 64 | 73 |
| hu119-30 | 69 | 74 |

TABLE 3
Combinations of the light and heavy chain variable
regions of the mAb179 humanized antibodies

| | VH (SEQ | VL (SEQ |
|---|---|---|
| Antibody | ID NO) | ID NO) |
| hu179-01 | 85 | 77 |
| hu179-02 | 85 | 78 |
| hu179-03 | 86 | 77 |
| hu179-04 | 86 | 78 |
| hu179-05 | 87 | 77 |
| hu179-06 | 87 | 78 |
| hu179-07 | 87 | 79 |
| hu179-08 | 87 | 81 |
| hu179-09 | 87 | 82 |
| hu179-10 | 87 | 83 |
| hu179-11 | 87 | 84 |
| hu179-12 | 88 | 77 |
| hu179-13 | 88 | 78 |
| hu179-14 | 89 | 79 |
| hu179-15 | 89 | 80 |
| hu179-16 | 89 | 81 |
| hu179-17 | 89 | 82 |
| hu179-18 | 89 | 83 |
| hu179-19 | 89 | 84 |
| hu179-20 | 90 | 78 |
| hu179-21 | 91 | 78 |
| hu179-22 | 92 | 78 |
| hu179-23 | 85 | 102 |
| hu179-24 | 85 | 104 |
| hu179-25 | 87 | 98 |
| hu179-26 | 87 | 99 |
| hu179-27 | 87 | 100 |
| hu179-28 | 87 | 101 |
| hu179-29 | 87 | 103 |
| hu179-30 | 87 | 105 |
| hu179-31 | 87 | 106 |
| hu179-32 | 87 | 107 |
| hu179-33 | 97 | 119 |

TABLE 4
Combinations of the light and heavy chain variable
regions of the mAb199 humanized antibodies

| | VH (SEQ | VL (SEQ |
|---|---|---|
| Antibody | ID NO) | ID NO) |
| hu199-01 | 127 | 120 |
| hu199-02 | 127 | 121 |
| hu199-03 | 127 | 122 |
| hu199-04 | 127 | 123 |
| hu199-05 | 127 | 124 |
| hu199-06 | 127 | 125 |
| hu199-07 | 128 | 120 |
| hu199-08 | 128 | 121 |
| hu199-09 | 128 | 122 |
| hu199-10 | 128 | 123 |
| hu199-11 | 128 | 124 |
| hu199-12 | 128 | 125 |
| hu199-13 | 129 | 120 |
| hu199-14 | 129 | 121 |
| hu199-15 | 129 | 122 |
| hu199-16 | 129 | 123 |
| hu199-17 | 129 | 124 |
| hu199-18 | 129 | 125 |
| hu199-19 | 130 | 120 |
| hu199-20 | 130 | 121 |
| hu199-21 | 130 | 122 |
| hu199-22 | 130 | 123 |
| hu199-23 | 130 | 124 |
| hu199-24 | 130 | 125 |
| hu199-25 | 131 | 120 |
| hu199-26 | 131 | 121 |
| hu199-27 | 131 | 122 |
| hu199-28 | 131 | 123 |
| hu199-29 | 131 | 124 |
| hu199-30 | 131 | 125 |
| hu199-31 | 132 | 120 |
| hu199-32 | 132 | 121 |
| hu199-33 | 132 | 122 |
| hu199-34 | 132 | 123 |
| hu199-35 | 132 | 124 |
| hu199-36 | 132 | 125 |

In some embodiments of the anti-TSLP antibody as described above, the antibody further comprises antibody constant region(s); preferably, the heavy chain constant region of the antibody constant regions is selected from the group consisting of human IgG1, IgG2, IgG3 and IgG4 constant regions and conventional variants thereof, the light chain constant region of the antibody constant regions is selected from the group consisting of human antibody κ and λ chain constant regions and conventional variants thereof, more preferably, the antibody comprises the heavy chain constant region as shown in sequence SEQ ID NO: 133, and the light chain constant region as shown in sequence SEQ ID NO: 134.

In some embodiments, the anti-TSLP antibody as described above comprises a heavy chain and a light chain as shown below:
(a) the amino acid sequence of the heavy chain is as shown in SEQ ID NO: 135 or has at least 90%, 92%, 94%, 95%, 96%, 97%, 98% or 99% sequence identity with the same, and the amino acid sequence of the light chain is as shown in SEQ ID NO: 136 or has at least 90%, 92%, 94%, 95%, 96%, 97%, 98% or 99% sequence identity with the same;
(b) the amino acid sequence of the heavy chain is as shown in SEQ ID NO: 137 or has at least 90%, 92%, 94%, 95%, 96%, 97%, 98% or 99% sequence identity with the same, and the amino acid sequence of the light chain is as shown in SEQ ID NO: 138 or has at least 90%, 92%, 94%, 95%, 96%, 97%, 98% or 99% sequence identity with the same;
(c) the amino acid sequence of the heavy chain is as shown in SEQ ID NO: 139 or has at least 90%, 92%, 94%, 95%, 96%, 97%, 98% or 99% sequence identity with the same, and the amino acid sequence of the light chain is as shown in SEQ ID NO: 140 or has at least 90% sequence identity with the same; or
(d) the amino acid sequence of the heavy chain is as shown in SEQ ID NO: 141 or has at least 90%, 92%, 94%, 95%, 96%, 97%, 98% or 99% sequence identity with the same, and the amino acid sequence of the light chain is as shown in SEQ ID NO: 142 or has at least 90%, 92%, 94%, 95%, 96%, 97%, 98% or 99% sequence identity with the same.

In some embodiments, the anti-TSLP antibody as described above comprises a heavy chain and a light chain as shown below:
(a) the amino acid sequence of the heavy chain is as shown in SEQ ID NO: 135, and the amino acid sequence of the light chain is as shown in SEQ ID NO: 136;
(b) the amino acid sequence of the heavy chain is as shown in SEQ ID NO: 137, and the amino acid sequence of the light chain is as shown in SEQ ID NO: 138;
(c) the amino acid sequence of the heavy chain is as shown in SEQ ID NO: 139, and the amino acid sequence of the light chain is as shown in SEQ ID NO: 140; or
(d) the amino acid sequence of the heavy chain is as shown in SEQ ID NO: 141, and the amino acid sequence of the light chain is as shown in SEQ ID NO: 142.

In some embodiments, the antibody competitively binds to human TSLP with the anti-TSLP antibody as described above or antigen-binding fragment thereof.

In another aspect, the present disclosure also provides a nucleic acid molecule encoding the anti-TSLP antibody as described above.

In another aspect, the present disclosure also provides an expression vector comprising the nucleic acid molecule as described above.

In another aspect, the present disclosure also provides a host cell comprising the nucleic acid molecule as described above or the expression vector as described above, preferably, the cell is a bacterial cell, a fungal cell, an insect animal cell or a mammalian cell.

In some embodiments, the present disclosure provides a method for preparing the TSLP antibody as described above.

In some embodiments, the present disclosure provides a pharmaceutical composition containing a therapeutically effective amount of the anti-TSLP antibody as described above, or the nucleic acid molecule as described above, or the host cell as described above, as well as one or more pharmaceutically acceptable carriers, diluents, buffers or excipients. Preferably, the therapeutically effective amount means 0.1-3000 mg or 1-1000 mg of the anti-TSLP antibody as described above contained in a unit dose of the composition.

In some embodiments, the present disclosure provides a method for immunodetection or determination of TSLP in vitro or ex vivo, which comprises a step of using the anti-TSLP antibody as described above.

In some embodiments, the present disclosure provides use of the anti-TSLP antibody as described above in preparing reagents for immunodetection of human TSLP.

In some embodiments, the present disclosure provides an anti-TSLP antibody as described above for use in immunodetection or determination of TSLP.

In some embodiments, the present disclosure provides a kit comprising the anti-TSLP antibody as described above.

In some embodiments, the present disclosure provides use of the anti-TSLP antibody as described above, or the nucleic acid molecule as described above, or the host cell as described above or the pharmaceutical composition as described above, in preparing a medicament for treating TSLP-related diseases: wherein the TSLP-related disease includes, but is not limited to: asthma, idiopathic pulmonary fibrosis, atopic dermatitis, allergic conjunctivitis, allergic rhinitis, allergic sinusitis, urticaria, Netherton syndrome, eosinophilic esophagitis, food allergy, allergic diarrhea, eosinophilic gastroenteritis, allergic bronchopulmonary aspergillosis, allergic fungal sinusitis, chronic pruritus, cancer, breast cancer, colon cancer, lung cancer, ovarian cancer, prostate cancer, rheumatoid arthritis, chronic obstructive pulmonary disease, systemic sclerosis, multiple sclerosis, keloidosis, ulcerative colitis, nasal polyposis, chronic eosinophilic pneumonia, eosinophilic bronchitis, celiac disease, Churg-Strauss syndrome, eosinophilic myalgia syndrome, hypereosinophilic syndrome, eosinophilic granulomatosis with polyangiitis, inflammatory bowel disease, scleroderma, interstitial lung disease, fibrosis caused by chronic hepatitis B or C, fibrosis induced by radiation and fibrosis caused by wound healing.

In some embodiments, the present disclosure provides a method for treating TSLP-related diseases, which comprises administering to a subject a therapeutically effective amount of the anti-TSLP antibody as described above, or the nucleic acid molecule as described above, or the host cell as described above or the pharmaceutical composition as described above; asthma, idiopathic pulmonary fibrosis, atopic dermatitis, allergic conjunctivitis, allergic rhinitis, allergic sinusitis, urticaria, Netherton syndrome, eosinophilic esophagitis, food allergy, allergic diarrhea, eosinophilic gastroenteritis, allergic bronchopulmonary aspergillosis, allergic fungal sinusitis, chronic pruritus, cancer, breast cancer, colon cancer, lung cancer, ovarian cancer, prostate cancer, rheumatoid arthritis, chronic obstructive pulmonary disease, systemic sclerosis, multiple sclerosis, keloidosis, ulcerative colitis, nasal polyposis, chronic eosinophilic pneumonia, eosinophilic bronchitis, celiac disease, Churg-Strauss syndrome, eosinophilic myalgia syndrome, hypereosinophilic syndrome, eosinophilic granulomatosis with polyangiitis, inflammatory bowel disease, scleroderma, interstitial lung disease, fibrosis caused by chronic hepatitis B or C, fibrosis induced by radiation and fibrosis caused by wound healing.

In some embodiments, the present disclosure provides an anti-TSLP antibody for use as a medicament, wherein the anti-TSLP antibody is for use in treating TSLP-related diseases, wherein the TSLP-related disease includes, but is not limited to: asthma, idiopathic pulmonary fibrosis, atopic dermatitis, allergic conjunctivitis, allergic rhinitis, allergic sinusitis, urticaria, Netherton syndrome, eosinophilic esophagitis, food allergy, allergic diarrhea, eosinophilic gastroenteritis, allergic bronchopulmonary aspergillosis, allergic fungal sinusitis, chronic pruritus, cancer, breast cancer, colon cancer, lung cancer, ovarian cancer, prostate cancer, rheumatoid arthritis, chronic obstructive pulmonary disease, systemic sclerosis, multiple sclerosis, keloidosis, ulcerative colitis, nasal polyposis, chronic eosinophilic pneumonia, eosinophilic bronchitis, celiac disease, Churg-Strauss syndrome, eosinophilic myalgia syndrome, hypereosinophilic syndrome, eosinophilic granulomatosis with polyangiitis, inflammatory bowel disease, scleroderma, interstitial lung disease, fibrosis caused by chronic hepatitis B or C, fibrosis induced by radiation and fibrosis caused by wound healing.

### DESCRIPTION OF THE DRAWINGS

FIG. 1: The result of the antibody blocking the binding activity of TSLP to TSLP receptor.

FIG. 2: The result of the antibody blocking the binding activity of TSLP to cell surface TSLP receptor.

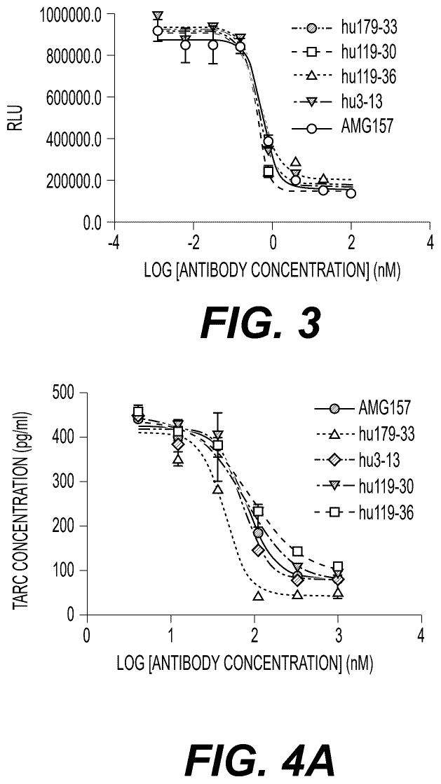

FIG. 3: The antibody inhibits TSLP-induced proliferation activity of BaF3 cells.

FIG. 4A shows the antibody activity of inhibiting the TSLP-induced production of the chemokine TARC; FIG. 4B shows the antibody activity of inhibiting the TSLP-induced production of the chemokine OPG.

FIG. 5A shows the antibody activity of inhibiting the production of the Th2 cytokine IL-13; FIG. 5B shows the antibody activity of inhibiting the production of the Th2 cytokine IL-4; FIG. 5C shows the antibody activity of inhibiting the production of the Th2 cytokine TNF-α; FIG. 5D shows the antibody activity of inhibiting the production of the Th2 cytokine IL-5.

### DETAILED DESCRIPTION OF THE DISCLOSURE

### Detailed Description of the Invention

### Terminology

To make the present disclosure easier to be understood, certain technical and scientific terms are specifically defined below. Unless clearly defined otherwise herein, all other technical and scientific terms used herein have the meanings commonly understood by those of ordinary skill in the art to which the present disclosure belongs.

The three-letter codes and one-letter codes of amino acids used in the present disclosure are as described in J. biol. chem, 243, p 3558 (1968).

The term “Thymic Stromal Lymphopoietin (TSLP)” is a type I cytokine with four α-helix bundle, and also known as an epithelial cell-derived cytokine produced in response to pro-inflammatory stimuli. It is closely related to interleukin-7 (IL-7), initiates allergic reactions by stimulating dendritic cells (DCs), and is an important factor in regulating the immune response in the human body. The term “TSLP” includes variants, isoforms, homologs, orthologs and paralogues of TSLP.

The “antibody” described in the present disclosure refers to an immunoglobulin, generally, the intact antibody is a tetrapeptide chain structure composed of two identical heavy chains and two identical light chains linked by interchain disulfide bonds. Immunoglobulin heavy chain constant regions exhibit different amino acid compositions and rank orders, hence present different antigenicity. Accordingly, immunoglobulins can be divided into five types, or named as immunoglobulin isotypes, namely IgM, IgD, IgG, IgA and IgE, and the corresponding heavy chains are μ chain, δ chain, γ chain, α chain and ε chain, respectively. The same type of Ig can be further divided into different subclasses according to the difference in the amino acid composition of the hinge region and the number and position of heavy chain disulfide bonds. For example, IgG can be divided into IgG1, IgG2, IgG3 and IgG4. The light chain is divided into κ chain or λ chain by the difference of the constant region. Each of the five types of Ig can have κ chain or λ chain.

The sequence of about 110 amino acids near the N-terminus of the antibody heavy and light chains varies greatly and known as the variable region (Fv region); the remaining amino acid sequence near the C-terminus is relatively stable and is the constant region. The variable region includes 3 hypervariable regions (HVR) and 4 framework regions (FR) with relatively conservative sequences. The 3 hypervariable regions determine the specificity of the antibody, and is also known as complementarity determining regions (CDR). Each light chain variable region (VL) and heavy chain variable region (VH) consists of 3 CDR regions and 4 FR regions. The order from the amino terminus to the carboxy terminus is: FR1, CDR1, FR2, CDR2, FR3, CDR3, FR4. The 3 CDR regions of the light chain refer to LCDR1, LCDR2 and LCDR3; the 3 CDR regions of the heavy chain refer to HCDR1, HCDR2 and HCDR3.

The antibodies of the present disclosure include murine antibodies, chimeric antibodies and humanized antibodies.

The term “murine antibody” in the present disclosure refers to a monoclonal antibody against human TSLP prepared according to the knowledge and skills in the art. During preparation, the test subject is injected with TSLP antigen, and then hybridomas expressing antibodies with the desired sequence or functional properties are isolated. In a preferred embodiment of the present disclosure, the murine anti-TSLP antibody or antigen-binding fragment thereof may further comprise a light chain constant region of murine κ, λ chain or variants thereof, or further comprise a heavy chain constant region of murine IgG1, IgG2, IgG3 or variants thereof.

The term “chimeric antibody” is an antibody formed by fusing the variable region of a murine antibody with the constant region of a human antibody, which can alleviate the immune response induced by murine antibody. Establishing a chimeric antibody requires first establishing a hybridoma secreting murine specific monoclonal antibodies, then cloning the variable region gene from the murine hybridoma cells, and then cloning the constant region gene of the human antibody as necessary, linking the murine variable region gene with the human constant region gene to form a chimeric gene to be inserted into an expression vector, and finally expressing the chimeric antibody molecule in a eukaryotic system or a prokaryotic system. In a preferred embodiment of the present disclosure, the antibody light chain of the TSLP chimeric antibody further comprises a light chain constant region of a human κ, λ, chain or variant thereof. The antibody heavy chain of the TSLP chimeric antibody further comprises the heavy chain constant region of human IgG1, IgG2, IgG3, IgG4 or variant thereof, preferably comprises the heavy chain constant region of human IgG1, IgG2 or IgG4, or IgG1, IgG2, or IgG4 variants with amino acid mutations (for example L234A and/or L235A mutations, and/or S228P mutations).

The term “humanized antibody”, also known as CDR-grafted antibody, refers to the antibody produced by grafting murine CDR sequences into the framework of human antibody variable regions, that is, an antibody produced in different types of human germline antibody framework sequences. It can overcome the heterogeneous reaction induced by the chimeric antibody as it carries a large amount of murine protein components. Such framework sequences can be obtained from public DNA databases or published references that include germline antibody gene sequences. For example, the germline DNA sequences of the human heavy chain and light chain variable region genes can be found in the “VBase” human germline sequence database (available on the Internet www.mrccpe.com.ac.uk/vbase), as well as in Kabat, E. A., et al., 1991, Sequences of Proteins of Immunological Interest, 5th edition. In order to avoid the decrease in activity at the same time caused by the decrease in immunogenicity, the human antibody variable region framework sequence can be subjected to minimal reverse mutations or back mutations to maintain activity. The humanized antibody of the present disclosure also includes humanized antibodies on which CDR affinity maturation is performed by yeast display.

CDR grafting may result in reduced affinity of the produced antibody or antigen-binding fragment thereof to the antigen due to changes of the framework residues in contact with the antigen. Such interactions may be the result of hypermutation of somatic cells. Therefore, it may still be necessary to graft such donor framework amino acids to the framework of the humanized antibody. The amino acid residues involved in antigen binding and from non-human antibodies or antigen-binding fragments thereof can be identified by examining the sequence and structure of the animal monoclonal antibody variable region. Residues in the CDR donor framework that differ from the germline can be considered related. If the closest germline cannot be determined, the sequence can be compared with the consensus sequence of a subclass or animal antibody sequence with a high percentage of similarity. Rare framework residues are thought to be the result of hypermutation of somatic cells and thus play an important role in binding.

In one embodiment of the present disclosure, the antibody or antigen-binding fragment thereof may further comprise the light chain constant region of human or murine κ, λ chain or variant thereof, or further comprise the heavy chain constant region of human or murine IgG1, IgG2, IgG3, IgG4 or variant thereof, preferably comprising the heavy chain constant region of human IgG1, IgG2 or IgG4, or IgG1, IgG2 or IgG4 variants with amino acid mutations (for example L234A/L235A mutation, S228P mutation, YTE mutation).

The “conventional variant” of the human antibody heavy chain constant region and the human antibody light chain constant region described in the present disclosure refer to the variant of heavy chain constant region or light chain constant region that has been disclosed in the prior art and does not change the structure and function of the antibody variable region. Exemplary variants include IgG1, IgG2, IgG3 or IgG4 heavy chain constant region variants with site-directed modifications and amino acid substitutions of the heavy chain constant region. Specific substitutions are such as YTE mutations, L234A and/or L235A mutations, S228P mutations, and/or mutations to obtain a knob-into-hole structure (making the antibody heavy chain have a combination of knob-Fc and hole-Fc) known in the art. These mutations have been confirmed to make the antibody have new properties, but does not change the function of the antibody variable region.

“Human antibody (HuMAb)”, “antibody derived from human”, “fully human antibody“and” completely human antibody” can be used interchangeably, and can be antibodies derived from humans or antibodies obtained from a genetically modified organism which has been “engineered” to produce specific human antibodies in response to antigen stimulation and can be produced by any method known in the art. In some technologies, the elements of human heavy chain and light chain gene loci are introduced into cell lines of organisms derived from embryonic stem cell lines, in which the endogenous heavy chain and light chain genetic loci are target disrupted. Transgenic organisms can synthesize human antibodies specific to human antigens, and the organisms can be used to produce human antibody-secreting hybridomas. A human antibody can also be an antibody in which the heavy and light chains are encoded by nucleotide sequences derived from one or more human DNA sources. A fully human antibody can also be constructed by gene or chromosome transfection methods and phage display technology, or constructed by B cells activated in vitro, all of which are known in the art.

The terms “full-length antibody”, “intact antibody”, “complete antibody” and “whole antibody” are used interchangeably herein and refer to an antibody in a substantially intact form, as distinguished from the antigen-binding fragments defined below. These terms specifically refer to an antibody whose light chain and heavy chain comprises constant region. The “antibody” of the present disclosure includes “full-length antibody” and antigen-binding fragments thereof.

In some embodiments, the full-length antibody of the present disclosure includes antibodies formed by linking the light chain variable region to the light chain constant region, and linking the heavy chain variable region to the heavy chain constant region, as shown in the light and heavy chain combination in the table 1 to 4 below. Those skilled in the art can select different antibody-derived light chain constant regions and heavy chain constant regions according to actual needs, for example, human antibody-derived light chain constant regions and heavy chain constant regions.

The term “antigen-binding fragment” or “functional fragment” of an antibody refers to one or more fragments of the antibody that retain the ability to specifically bind to an antigen (for example, TSLP). It has been shown that fragments of full-length antibodies can be used to perform the antigen-binding function of antibodies. Examples of the binding fragment included in the term “antigen-binding fragment” of an antibody include (i) Fab fragment, a monovalent fragment consisting of VL, VH, CL and CH1 domains; (ii) F(ab′)₂ fragment, a bivalent fragment comprising two Fab fragments linked by a disulfide bridge in the hinge region; (iii) Fd fragment, consisting of VH and CH1 domains; (iv) Fv fragment, consisting of VH and VL domains of one arm of the antibody; (V) dsFv, a stable antigen-binding fragment formed by interchain disulfide bonds between VH and VL; (vi) diabody, bispecific antibody and multispecific antibody, comprising fragments like scFv, dsFv, Fab, etc. In addition, although the two domains VL and VH of the Fv fragment are encoded by separate genes, recombination methods can be used to link them by synthetic linkers so that it can be produced as a single protein chain in which the VL and VH regions pair to form a monovalent molecule (referred to as single-chain Fv (scFv); see, for example, Bird et al. (1988) Science 242:423-426; and Huston et al. (1988) Proc. Natl. Acad. Sci USA 85: 5879-5883). Such single chain antibodies are also included in the term “antigen-binding fragment” of an antibody. Such antibody fragments are obtained by using conventional techniques known to those skilled in the art, and screened in the same manner as that used for intact antibodies. The antigen binding moiety can be produced by recombinant DNA technology or by enzymatic or chemical fragmentation of the intact immunoglobulin. The antibodies may be antibodies of different isotypes, for example, IgG (for example, IgG1, IgG2, IgG3 or IgG4 subtypes), IgA1, IgA2, IgD, IgE or IgM antibodies.

Fab is an antibody fragment that has a molecular weight of about 50,000 and has antigen-binding activity among fragments obtained by treating IgG antibody molecules with papain (which cleaves the amino acid residue at position 224 of the H chain), in which about half of the H chain of the N-terminal side and the entire L chain are joined together by disulfide bonds.

F(ab′)2 is an antibody fragment that has a molecular weight of about 100,000 and has antigen-binding activity and comprises two Fab regions connected at the hinge position among fragments obtained by digesting the lower part of the two disulfide bonds in the hinge region of IgG with the enzyme pepsin.

Fab′ is an antibody fragment that has a molecular weight of about 50,000 and has antigen-binding activity obtained by cleaving the disulfide bond in the hinge region of the F(ab′)2. The Fab′ of the present disclosure can be produced by using reducing agents, for example dithiothreitol, to treat the F(ab′)2 of the present disclosure which specifically recognizes TSLP and binds to the amino acid sequence of the extracellular domain or three-dimensional structure thereof.

In addition, the Fab′ can be produced by inserting the DNA encoding the Fab′ fragment of the antibody into a prokaryotic expression vector or a eukaryotic expression vector and introducing the vector into a prokaryotic organism or eukaryotic organism to express the Fab′.

The term “single-chain antibody”, “single-chain Fv” or “scFv” refers to molecules comprising an antibody heavy chain variable domain (or region, VH) and an antibody light chain variable domain (or region, VL) connected by a linker. Such scFv molecules can have the general structure: NH₂-VL-linker-VH-COOH or NH₂-VH-linker-VL-COOH. Suitable prior art linkers consist of repeated GGGGS amino acid sequences or variants thereof, for example using 1 to 4 repeated variants (Holliger et al. (1993), Proc. Natl. Acad. Sci. USA 90: 6444-6448). Other linkers that can be used in the present disclosure are described in Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol.

Diabody is an antibody fragment in which scFv or Fab is dimerized, and is an antibody fragment with bivalent antigen-binding activity. In the bivalent antigen binding activity, the two antigens can be the same or different.

Bispecific antibody and multispecific antibody refer to an antibody that can simultaneously bind to two or more antigens or antigenic determinants, including scFv or Fab fragments that can bind to TSLP.

The diabody of the present disclosure can be produced by the following steps: obtaining the coding cDNA of VH and VL of the monoclonal antibody of the present disclosure which specifically recognizes human TSLP and binds to the amino acid sequence of the extracellular domain or three-dimensional structure thereof, constructing the DNA encoding scFv so that the amino acid sequence length of the peptide linker is 8 residues or less, inserting the DNA into a prokaryotic expression vector or a eukaryotic expression vector, and then introducing the expression vector into a prokaryote organism or eukaryotic organism to express the diabody.

dsFv is obtained by linking VH and VL polypeptides in which one amino acid residue in each is substituted with a cysteine residue via disulfide bonds between the cysteine residues. The amino acid residues substituted with cysteine residues can be selected according to known methods (Protein Engineering, 7, 697 (1994)) based on the three-dimensional structure prediction of the antibody.

The full-length antibody or antigen-binding fragment of the present disclosure can be produced by the following steps: obtaining the coding cDNA of VH and VL of the monoclonal antibody of the present disclosure which specifically recognizes human TSLP and binds to the amino acid sequence of the extracellular domain or three-dimensional structure thereof, constructing the DNA encoding the full-length antibody or antigen-binding fragment, inserting the DNA into a prokaryotic expression vector or a eukaryotic expression vector, and then introducing the expression vector into a prokaryotic organism or eukaryotic organism for expression.

The term “amino acid difference” or “amino acid mutation” refers to the presence of amino acid changes or mutations in the variant protein or polypeptide compared with the original protein or polypeptide, including occurrence of 1, 2, 3 or more amino acid insertion, deletion or substitution on the basis of the original protein or polypeptide.

The term “antibody framework” or “FR region” refers to a moiety of the variable domain VL or VH, which serves as a scaffold for the antigen binding loop (CDR) of the variable domain. Essentially, it is a variable domain without CDR.

The term “complementarity determining region”, “CDR” or “hypervariable region” refers to one of the six hypervariable regions in the variable domain of an antibody that mainly contribute to antigen binding. Generally, there are three CDRs (HCDR1, HCDR2, HCDR3) in each heavy chain variable region, and three CDRs (LCDR1, LCDR2, LCDR3) in each light chain variable region. Any one of a variety of well-known schemes can be used to determine the amino acid sequence boundaries of the CDRs, including the “Kabat” numbering rules (see Kabat et al. (1991), “Sequences of Proteins of Immunological Interest”, 5th edition, Public Health Service, National Institutes of Health, Bethesda, MD), “Chothia” numbering rules (see Al-Lazikani et al., (1997) JMB 273: 927-948) and ImmunoGenTics (IMGT) numbering rules (Lefranc M. P., Immunologist, 7, 132-136 (1999); Lefranc, M. P., et al., Dev. Comp. Immunol., 27, 55-77 (2003)), etc. For example, for the classical format, following the Kabat rule, the amino acid residue numbers of CDRs in the heavy chain variable domain (VH) are 31-35 (HCDR1), 50-65 (HCDR2) and 95-102 (HCDR3); the amino acid residue numbers of CDRs in the light chain variable domain (VL) are 24-34 (LCDR1), 50-56 (LCDR2) and 89-97 (LCDR3). Following the Chothia rule, the amino acid residue numbers of CDRs in VH are 26-32 (HCDR1), 52-56 (HCDR2) and 95-102 (HCDR3); and the amino acid residue numbers in VL are 26-32 (LCDR1), 50-52 (LCDR2) and 91-96 (LCDR3). By combining the CDR definitions of both Kabat and Chothia, the CDRs consist of amino acid residues 26-35 (HCDR1), 50-65 (HCDR2) and 95-102 (HCDR3) in human VH and amino acid residues 24-34 (LCDR1), 50-56 (LCDR2) and 89-97 (LCDR3) in human VL. Following IMGT rules, the amino acid residue numbers of CDRs in VH are roughly 26-35 (CDR1), 51-57 (CDR2) and 93-102 (CDR3), and the amino acid residue numbers of CDRss in VL are roughly 27-32 (CDR1), 50-52 (CDR2) and 89-97 (CDR3). Following IMGT rules, the CDR regions of an antibody can be determined by using the program IMGT/DomainGap Align.

The term “epitope” or “antigenic determinant” refers to a site on an antigen where an immunoglobulin or antibody specifically binds (for example, a specific site on TSLP molecules). Epitopes generally include at least 3, 4, 5, 6, 7, 8, 9, 10, 11, 12, 13, 14 or 15 consecutive or non-consecutive amino acids in a unique spatial conformation. See, for example, Epitope Mapping Protocols in Methods in Molecular Biology, Vol. 66, G. E. Morris, Ed. (1996).

The terms “specifically binds”, “selectively binds”, “binds selectively” and “binds specifically” refer to the binding of an antibody to an epitope on a predetermined antigen. Generally, an antibody binds with an affinity (KD) of about less than 10⁻⁸M, for example about less than 10⁻⁹M, 10⁻¹⁰M, 10⁻¹¹M, 10⁻¹²M or less.

The term “KD” refers to the dissociation equilibrium constant of a specific antibody-antigen interaction. Generally, the antibody of the present disclosure binds to TSLP with an affinity (KD) of about less than 10⁻⁷M, for example about less than 10⁻⁸M or 10⁻⁹M, for example, in the present disclosure, the affinity of the antibody to the cell surface antigen is determined by the FACS or Biacore method to determine the KD value.

When the term “competition” is used in the context of antigen-binding proteins (for example neutralizing antigen-binding protein or neutralizing antibody) that compete for the same epitope, it refers to the competition between the antigen-binding proteins, which is determined by the following assay: in the assay, the antigen-binding proteins to be tested (for example antibodies or immunological functional fragments thereof) prevent or inhibit (for example reduce) the specific binding of a reference antigen-binding protein (for example a ligand or a reference antibody) to a common antigen (for example TSLP antigen or fragment thereof). Numerous types of competitive binding assays can be used to determine whether one antigen-binding protein competes with another, these assays are for example: solid phase direct or indirect radioimmunoassay (RIA), solid phase direct or indirect enzyme immunoassay (EIA), sandwich competition assay (see for example Stahli et al., 1983, Methods in Enzymology 9:242-253); solid phase direct biotin-avidin EIA (see for example Kirkland et al., 1986, J. Immunol. 137:3614-3619), solid phase direct labeling assay, solid phase direct labeling sandwich assay (see for example Harlow and Lane, 1988, Antibodies, A Laboratory Manual, Cold Spring Harbor Press); solid phase direct labeling RIA with I-125 labels (see for example Morel et al., 1988, Molec. Immunol. 25: 7-15); solid-phase direct biotin-avidin EIA (see for example Cheung, et al., 1990, Virology 176: 546-552); and directly labeling RIA (Moldenhauer et al., 1990, Scand. J. Immunol. 32:77-82). Generally, the assays involve using any one of unlabeled test antigen-binding protein and labeled reference antigen-binding protein to bind purified antigens bound to a solid surface or cells. Competitive inhibition is measured by measuring the amount of label bound to the solid surface or cells in the presence of the test antigen-binding protein. Generally, the test antigen-binding protein is present in excess. The antigen-binding proteins identified by competition assays (competitive antigen-binding proteins) include: antigen-binding proteins that bind to the same epitope as the reference antigen-binding protein; and antigen-binding proteins that binds to adjacent epitopes that are sufficiently close to the binding epitope of the reference antigen-binding protein, the two epitopes sterically hindering each other from binding. Generally, when the competitive antigen-binding protein is present in excess, it will inhibit (for example reduce) the specific binding of the reference antigen-binding protein to the common antigen by at least 40-45%, 45-50%, 50-55%, 55-60%, 60-65%, 65-70%, 70%-75% or 75% or more. In some cases, the binding is inhibited by at least 80-85%, 85-90%, 90-95%, 95-97%, or 97% or more.

The term “nucleic acid molecule” used herein refers to DNA molecule and RNA molecule. The nucleic acid molecule can be single-stranded or double-stranded, and is preferably double-stranded DNA or single-stranded mRNA or modified mRNA. When a nucleic acid is placed in a functional relationship with another nucleic acid sequence, the nucleic acid is “operably linked”. For example, if a promoter or enhancer affects the transcription of a coding sequence, then the promoter or enhancer is operably linked to the coding sequence.

Amino acid sequence “identity” “identity” refers to the percentage of the amino acid residues that are identical between the first and the second sequence when the amino acid sequences are aligned (introducing gaps when necessary) to achieve the maximum percentage of sequence identity, and no conservative substitutions are considered as part of the sequence identity. For the purpose of determining the percentage of amino acid sequence identity, the alignment can be achieved by a variety of ways within the technical scope of the art, for example, using publicly available computer software, such as BLAST, BLAST-2, ALIGN, ALIGN-2 or Megalign (DNASTAR) software. Those skilled in the art can determine the parameters suitable for measuring the alignment, including any algorithm required to achieve the maximum alignment over the entire length of the sequences being compared.

The term “expression vector” refers to a nucleic acid molecule capable of transporting another nucleic acid to which it has been linked. In one embodiment, the vector is a “plasmid”, which refers to a circular double-stranded DNA loop into which additional DNA segments can be linked. In another embodiment, the vector is a viral vector in which additional DNA segments can be linked into the viral genome. The vectors disclosed herein can replicate autonomously in the host cell into which they have been introduced (for example, bacterial vectors with bacterial origin of replication and episomal mammalian vectors) or can be integrated into the genome of the host cell after being introduced into the host cell, so as to replicate together with the host genome (for example, non-episomal mammalian vectors).

The methods for producing and purifying antibodies and antigen-binding fragments are well known in the prior art, such as Antibody Experiment Technical Guide, Cold Spring Harbor, Chapters 5-8 and 15. For example, mice can be immunized with human TSLP or fragment thereof, and the obtained antibodies can be renatured and purified, and amino acid sequencing can be performed by using conventional methods. Antigen-binding fragments can also be prepared by using conventional methods. The antibody or antigen-binding fragment according to the present disclosure is genetically engineered to add one or more human FR regions to the non-human CDR regions. The human FR germline sequences can be obtained from the ImmunoGeneTics (IMGT) website http://imgt.cines.fr by comparing the IMGT human antibody variable region germline gene database and MOE software, or be obtained from The Immunoglobulin FactsBook, 2001ISBN012441351.

The term “host cell” refers to a cell into which an expression vector has been introduced. Host cells can include bacteria, microorganisms, plant or animal cells. Bacteria that can be easily transformed include members of the enterobacteriaceae, for example Escherichia coli or Salmonella strains; Bacillaceae, for example Bacillus subtilis; Pneumococcus; Streptococcus and Haemophilus influenzae. Suitable microorganisms include Saccharomyces cerevisiae and Pichia pastoris. Suitable animal host cell lines include CHO (Chinese Hamster Ovary Cell Line), 293 cells and NS0 cells.

The engineered antibodies or antigen-binding fragments of the present disclosure can be prepared and purified by conventional methods. For example, the cDNA sequences encoding the heavy chain and light chain can be cloned and recombined into a GS expression vector. The recombinant immunoglobulin expression vectors can stably transfect CHO cells. As a more recommended prior art, mammalian expression systems can lead to glycosylation of antibodies, especially in the highly conserved N-terminal sites of the Fc region. Stable clones are obtained by expressing antibodies that specifically bind to human TSLP. Positive clones are expanded in serum-free medium of bioreactors to produce antibodies. The medium into which the antibodies are secreted can be purified by conventional techniques. For example, using A or G Sepharose FF column with adjusted buffer for purification. Non-specifically bound components are washed off. Then the bound antibodies are eluted by the pH gradient method, and the antibody fragments are detected by SDS-PAGE and collected. The antibodies can be filtered and concentrated by conventional methods. Soluble mixtures and polymers can also be removed by conventional methods, for example molecular sieves and ion exchange. The resulting product needs to be frozen immediately, such as at −70° C., or lyophilized.

“Administering”, “giving” and “treating”, when applied to animals, humans, experimental subjects, cells, tissues, organs or biological fluids, refer to the contact of the exogenous medicament, therapeutic agent, diagnostic agent or composition with the animals, humans, subjects, cells, tissues, organs or biological fluids. “Administering”, “giving” and “treating” can refer to for example treatment, pharmacokinetics, diagnosis, research and experimental methods. Treating cells includes contacting reagents with cells, and contacting reagents with fluids, wherein the fluids are in contact with the cells. “Administering”, “giving” and “treating” also refer to treating for example cells by reagents, diagnosis, binding compositions or by another cell in vitro and ex vivo. “Treating” when applied to human, veterinary or research subjects, refers to therapeutic treatment, preventive or prophylactic measures, research and diagnostic applications.

“Treatment” refers to giving an internal or external therapeutic agent, for example a composition comprising any one of the binding compounds of the present disclosure, to a patient with one or more disease symptoms on which the therapeutic agent is known to have therapeutic effect. Generally, the therapeutic agent is given at an amount effective to alleviate one or more disease symptoms in the patient or population treated to induce the regression of such symptoms or inhibit the development of such symptoms to any clinically measurable extent. The amount of therapeutic agent that is effective to alleviate any specific disease symptom (also referred to as a “therapeutically effective amount”) can vary according to a variety of factors, for example the patient's disease state, age and body weight, and the ability of the drug to produce the desired therapeutic effect in the patient. Whether the disease symptoms have been alleviated can be evaluated by any clinical testing methods commonly used by doctors or other health care professionals to evaluate the severity or progression of the symptoms. Although the embodiments of the present disclosure (for example treatment methods or products) may not be effective in alleviating the target disease symptom(s) in every patient, as determined according to any statistical testing methods known in the art, such as Student t-test, chi-square test, Mann and Whitney's U test, Kruskal-Wallis test (H test), Jonckheere-Terpstra test and Wilcoxon test, they should reduce the target disease symptom(s) in a statistically significant number of patients.

“Conservative modification” or “conservative replacement or substitution” refers to substitutions of amino acids in a protein with other amino acids having similar characteristics (e.g. charge, side-chain size, hydrophobicity/hydrophilicity, backbone conformation and rigidity, etc.), so that changes can be frequently made without changing the biological activity of the protein. Those skilled in the art know that, generally speaking, a single amino acid substitution in a non-essential region of a polypeptide does not substantially change the biological activity (see for example Watson et al. (1987) Molecular Biology of the Gene, The Benjamin/Cummings Pub. Co., Page 224, (4th edition)). In addition, the substitution of amino acids with similar structure or function is unlikely to disrupt the biological activity. Exemplary conservative substitutions are stated in the table “Exemplary amino acid conservative substitutions” below.

TABLE 5
Exemplary amino acid conservative substitutions

| Original residue | Conservative substitution |
|---|---|
| Ala (A) | Gly; Ser |
| Arg (R) | Lys; His |
| Asn (N) | Gln; His; Asp |
| Asp (D) | Glu; Asn |
| Cys (C) | Ser; Ala; Val |
| Gln (Q) | Asn; Glu |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Effective amount” or “effective dose” refers to the amount of a drug, compound or pharmaceutical composition necessary to obtain any one or more beneficial or desired therapeutic results. For preventive use, the beneficial or desired results include elimination or reduction of risk, reduction of severity or delay of the disease onset, including the biochemistry, histology and/or behavioral symptoms of the disease, complications thereof and intermediate pathological phenotypes that appear during the developmental process of the disease. For therapeutic applications, the beneficial or desired results include clinical results, such as reducing the incidence of various target antigen-related disorders of the present disclosure or improving one or more symptoms of the disorder, reducing the dose of other agents required to treat the disorder, enhancing the therapeutic effect of another agent, and/or delaying the progression disorders of the patient related to the target antigen of the present disclosure.

“Exogenous” refers to substances produced outside organisms, cells or human bodies according to circumstances. “Endogenous” refers to substances produced inside cells, organisms or human bodies according to circumstances.

“Homology” refers to the sequence similarity between two polynucleotide sequences or between two polypeptides. When the positions in the two sequences compared are occupied by the same base or amino acid monomer subunit, for example if each position of two DNA molecules is occupied by adenine, then the molecules are homologous at that position. The homology percentage between two sequences is a function of the number of matched or homologous positions shared by the two sequences divided by the number of positions compared ×100. For example, in the optimal sequence alignment, if 6 out of 10 positions in the two sequences are matched or homologous, then the two sequences are 60% homologous; if 95 out of 100 positions in the two sequences are matched or homologous, then the two sequences are 95% homologous. Generally, when aligning two sequences, comparisons are made to give the maximum percentage homology. For example, the comparison can be performed by the BLAST algorithm, wherein the parameters of the algorithm are selected to give the maximum match between each sequence over the entire length of each reference sequence. The following references relate to the BLAST algorithm that is often used for sequence analysis: BLAST ALGORITHMS: Altschul, S. F. et al., (1990) J. Mol. Biol. 215:403-410; Gish, W. et al., (1993) Nature Genet. 3:266-272; Madden, T. L. et al., (1996) Meth. Enzymol. 266:131-141; Altschul, S. F. et al., (1997) Nucleic Acids Res. 25:3389-3402; Zhang, J. et al., (1997) Genome Res. 7:649-656. Other conventional BLAST algorithms, such as those provided by NCBI BLAST, are also well known to those skilled in the art.

The expressions “cell”, “cell line” and “cell culture” as used herein can be used interchangeably, and all such names include the progeny. Therefore, the words “transformant” and “transformed cell” include primary test cells and cultures derived therefrom, regardless of the number of passages. It should also be understood that due to deliberate or unintentional mutations, all offspring cannot be exactly the same in terms of DNA content. Mutant progeny with the same function or biological activity as screened in the original transformed cells is included. It is clearly visible from the context when a different name is referred to.

“Polymerase chain reaction” or “PCR” as used herein refers to a procedure or technique in which a trace amount of a specific moiety of nucleic acid, RNA and/or DNA is amplified as described in, for example, U.S. Pat. No. 4,683,195. Generally speaking, it is necessary to obtain sequence information from the end or outside of the target region, so that oligonucleotide primers can be designed; these primers are the same or similar in terms of sequence to the corresponding strand of the template to be amplified. The 5′ terminal nucleotides of the two primers can be identical to the ends of the material to be amplified. PCR can be used to amplify specific RNA sequences, specific DNA sequences from total genomic DNA and cDNA sequences transcribed from total cellular RNA, phage or plasmid sequences, etc. Generally, see Mullis et al. (1987) Cold Spring Harbor, Symp. Ouant. Biol. 51:263; Erlich ed., (1989) PCR TECHNOLOGY (Stockton Press, N.Y.). The PCR used herein is regarded as an example, but not the only example, of a nucleic acid polymerase reaction method for amplifying a nucleic acid test sample, and the method includes using known nucleic acids as primers and nucleic acid polymerases to amplify or produce a specific moiety of the nucleic acid.

“Isolated” refers to a purified state, and in this case means that the designated molecule is substantially free of other biomolecules, for example nucleic acids, proteins, lipids, carbohydrates or other materials, for example cell debris and growth medium. Generally, the term “isolated” is not intended to mean the complete absence of these materials or the absence of water, buffer or salt, unless they are present in an amount that significantly interferes with the experimental or therapeutic use of the compound as described herein.

“Optional” or “optionally” means that the event or environment described later can occur, but does not have to occur, and this description includes occasions where the event or environment occurs or does not occur.

“Pharmaceutical composition” means a mixture containing one or more of the compounds described herein or physiologically/pharmaceutically acceptable salts or prodrugs thereof, and other chemical components, for example physiological/pharmaceutically acceptable carriers and excipients. The purpose of the pharmaceutical composition is to promote the administration to organisms, which facilitates the absorption of the active ingredient and thereby exerts biological activity.

The term “pharmaceutically acceptable carrier” refers to any inactive substance suitable for use in a formulation for the delivery of antibodies or antigen-binding fragments. The carrier can be an anti-adhesive agent, binder, coating, disintegrant, filler or diluent, preservative (such as antioxidant, antibacterial or antifungal agent), sweetener, absorption delaying agent, wetting agent, emulsifier, buffer, etc. Examples of suitable pharmaceutically acceptable carriers include water, ethanol, polyol (for example glycerol, propanediol, polyethylene glycol, etc.), dextrose, vegetable oil (for example olive oil), saline, buffer, buffered saline, and isotonic agent for example sugar, polyol, sorbitol and sodium chloride.

In addition, the present disclosure includes agents for treating TSLP-related diseases, comprising the anti-TSLP antibody of the present disclosure or antigen-binding fragment thereof as an active ingredient.

There is no limitation for the TSLP-related disease in the present disclosure, as long as it is a disease related to TSLP. For example, the therapeutic response induced by the molecule of the present disclosure can be achieved by binding to human TSLP, and then blocking the binding of TSLP to its receptors, or killing cells overexpressing TSLP.

In addition, the present disclosure relates to methods for immunodetection or determination of the target antigen (for example TSLP), reagents for immunodetection or determination of the target antigen (for example TSLP), methods for immunodetection or determination of cells expressing the target antigen (for example TSLP) and diagnostic agents for diagnosing diseases related to target antigen (for example TSLP) positive cells, which includes the antibody or antibody fragment of the present disclosure as active ingredient, which specifically recognizes the target antigen (for example human TSLP) and binds with the amino acid sequence of the extracellular domain or three-dimensional structure thereof.

In the present disclosure, the method used for detection or measurement of the amount of the target antigen (for example TSLP) may be any known method. For example, it includes immunodetection or measurement methods.

The immunodetection or measurement methods are methods of detecting or measuring the amount of antibody or antigen using labeled antigens or antibodies. Examples of immunodetection or measurement methods include radioimmunoassay (RIA), enzyme immunoassay (EIA or ELISA), fluorescence immunoassay (FIA), luminescence immunoassay, western blotting, physicochemical methods, etc.

The aforementioned TSLP-related diseases can be diagnosed by detecting or measuring cells expressing TSLP by using the antibody or antibody fragment of the present disclosure.

In order to detect cells expressing the polypeptide, known immunodetection methods can be used, preferably using immunoprecipitation, fluorescent cell staining, immunohistochemical staining, etc. In addition, fluorescent antibody staining method utilizing the FMAT8100HTS system (Applied Biosystem) can be used.

In the present disclosure, there is no particular limitation for the in vivo sample used for detection or measurement of the target antigen (for example TSLP), as long as it has the possibility of comprising cells expressing the target antigen (for example TSLP), for example histocyte, blood, plasma, serum, pancreatic juice, urine, feces, tissue fluid or culture fluid.

According to the required diagnostic method, the diagnostic agent containing the monoclonal antibody or antibody fragment thereof of the present disclosure can also contain reagents for performing antigen-antibody reaction or reagents for detecting the reaction. The reagents used for performing the antigen-antibody reaction include buffers, salts, etc. The reagents used for detection include reagents commonly used in immunodetection or measurement methods, for example labeled second antibodies that recognize the monoclonal antibody, antibody fragment thereof or conjugate thereof, and substrates corresponding to the label, etc.

In the above specification, presented are the details of one or more embodiments of the present disclosure. Although any methods and materials similar or identical to those described herein can be used to implement or test the present invention, the preferred methods and materials are described below. The other features, purposes and advantages of the present disclosure will be obvious through the specification and the claims. In the specification and the claims, unless otherwise clearly indicated in the context, the singular form includes the cases of plural referent. Unless otherwise defined, all technical and scientific terms used herein have the general meanings understood by those of ordinary skill in the art to which the present invention belongs. All patents and publications cited in the specification are incorporated by reference. The following examples are presented to more comprehensively illustrate the preferred embodiments of the present invention. These examples should not be construed as limiting the scope of the present invention in any way, and the scope of the present invention is defined by the claims.

### EXAMPLES

The examples below are incorporated for further description of the present disclosure, but these examples do not limit the scope of the present disclosure.

The experimental methods with unspecified conditions in the examples or test examples of the present disclosure generally follow conventional conditions, or according to the conditions recommended by the raw material or commodity manufacturer. See Sambrook et al., Molecular Cloning: A Laboratory Manual, Cold Spring Harbor; Current Protocols Molecular Biology, Ausubel et al., Greene Publishing Associates, Wiley Interscience, NY. The reagents with unspecified sources are conventional reagents purchased on the market.

### Example 1. Expression of TSLP and TSLP Receptor

The sequences encoding His-tagged human TSLP and cyno TSLP, human IgG1-Fc-tagged human TSLP and cyno TSLP, and TSLP receptor extracellular domain sequences were loaded onto phr vector to construct expression plasmids, which were then transfected into HEK293. The specific transfection steps were as follows: on the previous day, HEK293E cells were seeded in Freestyle expression medium (containing 1% FBS) at 0.8×10⁶/ml, placed on a 37° C. constant temperature shaker (120 rpm) and continued culturing for 24 hours. After 24 hours, the transfection plasmid and the transfection reagent PEI were sterilized with 0.22 μm filters. Then the transfection plasmid was adjusted to 100 μg/100 ml cells, and the mass ratio of PEI (1 mg/ml) and plasmid was 3:1. Taking the transfection of 200 ml HEK293E cells as an example, 10 ml of Opti-MEM and 200 μg plasmid were taken and mixed well, and let stand for 5 min; another 10 ml of Opti-MEM and 600 μg PEI were taken and mixed well, and let stand for 5 min. The plasmid and PEI were mixed well and let stand for 15 min, better not exceeding 20 min. The mixture of the plasmid and PEI was slowly added to 200 ml HEK293E cells, and placed on a shaker at 8% CO₂, 120 rpm and 37° C. for culturing. On day 3 of transfection, the culture was supplemented with 10% volume of supplemented medium. Until day 6 of transfection, samples were taken and centrifuged at 4500 rpm for 10 min to collect the cell supernatant. The supernatant was filtered and purified to obtain the recombinant TSLP and TSLP receptor proteins through Example 2. The purified proteins could be used in the experiments of each example below. The relevant sequences are as follows.

1. Amino Acid Sequence of His-Tagged Human TSLP Antigen (huTSLP-his)

SEQ ID NO: 1
MFPFALLYVLSVSFRKIFILQLVGLVLTYDFTNCDFEKIKAAYLS
TISKDLITYMSGTKSTEFNNTVSCSNRPHCLTEIQSLTFNPTAGC
ASLAKEMFAMKTKAALAIWCPGYSETQINATQAMKKARKSKVTTN
KCLEQVSQLQGLWRRFNRPLLKQQGSSDYKDDDDKHHHHHH
Note:
Underlined is the signal peptide sequence;
the italicized part is the Flag-His6-tag.

2. Amino Acid Sequence of Fc-Tagged Human TSLP Antigen (huTSLP-Fc)

SEQ ID NO: 2
MFPFALLYVLSVSFRKIFILQLVGLVLTYDFTNCDFEKIKAAYLS
TISKDLITYMSGTKSTEFNNTVSCSNRPHCLTEIQSLTFNPTAGC
ASLAKEMFAMKTKAALAIWCPGYSETQINATQAMKKARKSKVTTN
KCLEQVSQLQGLWRRFNRPLLKQQDIEGRMDEPKSSDKTHTCPPC
PAPELLGGPSVFLFPPKPKDTLMISRTPEVTCVVVDVSHEDPEVK
FNWYVDGVEVHNAKTKPREEQYNSTYRVVSVLTVLHQDWLNGKEY
KCKVSNKALPAPIEKTISKAKGQPREPQVYTLPPSRDELTKNQVS
LTCLVKGFYPSDIAVEWESNGQPENNYKTTPPVLDSDGSFFLYSK
LTVDKSRWQQGNVFSCSVMHEALHNHYTQKSLSLSPGK
Note:
Underlined is the signal peptide sequence;
the italicized part is the linker-human Fc-tag.

3. Amino Acid Sequence of His-Tagged Cyno TSLP Antigen (cynoTSLP-his)

SEQ ID NO: 3
METDTLLLWVLLLWVPGSTGYDFTNCDFQKIEADYLRTISKDLIT
YMSGTKSTDFNNTVSCSNRPHCLTEIQSLTFNPTPRCASLAKEMF
ARKTKATLALWCPGYSETQINATQAMKKARKSKVTTNKCLEQVSQ
LLGLWRRFIRTLLKQQGSSDYKDDDDKHHHHHH
Note:
Underlined is the signal peptide sequence;
the italicized part is the flag-His6-tag.

4. Amino Acid Sequence of Fc-Tagged Cyno TSLP Antigen (cynoTSLP-Fc)

SEQ ID NO: 4
METDTLLLWVLLLWVPGSTGYDFTNCDFQKIEADYLRTISKDLIT
YMSGTKSTDFNNTVSCSNRPHCLTEIQSLTFNPTPRCASLAKEMF
ARKTKATLALWCPGYSETQINATQAMKKARKSKVTTNKCLEQVSQ
LLGLWRRFIRTLLKQQDIEGRMDEPKSSDKTHTCPPCPAPELLGG
PSVFLFPPKPKDTLMISRTPEVTCVVVDVSHEDPEVKFNWYVDGV
EVHNAKTKPREEQYNSTYRVVSVLTVLHQDWLNGKEYKCKVSNKA
LPAPIEKTISKAKGQPREPQVYTLPPSRDELTKNQVSLTCLVKGF
YPSDIAVEWESNGQPENNYKTTPPVLDSDGSFFLYSKLTVDKSRW
QQGNVFSCSVMHEALHNHYTQKSLSLSPGK
Note:
Underlined is the signal peptide sequence;
the italicized part is the linker-human Fc-
tag.

### 5. Amino Acid Sequence of Fc-Tagged Human TSLP Receptor Extracellular Domain (Human-TSLPR-Fc-ECD):

SEQ ID NO: 5
GAAEGVQIQIIYFNLETVQVTWNASKYSRTNLTFHYRFNGDEAY
DQCTNYLLQEGHTSGCLLDAEQRDDILYFSIRNGTHPVFTASRW
MVYYLKPSSPKHVRFSWHQDAVTVTCSDLSYGDLLYEVQYRSPE
DTEWQSKQENTCNVTIEGLDAEKCYSFWVRVKAMEDVYGPDTYP
SDWSEVTCWQRGEIRDACAETPTPPKPKLSKDIEGRMDEPKSSD
KTHTCPPCPAPELLGGPSVFLFPPKPKDTLMISRTPEVTCVVVD
VSHEDPEVKFNWYVDGVEVHNAKTKPREEQYNSTYRVVSVLTVL
HQDWLNGKEYKCKVSNKALPAPIEKTISKAKGQPREPQVYTLPP
SRDELTKNQVSLTCLVKGFYPSDIAVEWESNGQPENNYKTTPPV
LDSDGSFFLYSKLTVDKSRWQQGNVFSCSVMHEALHNHYTQKSL
SLSPGK
Note:
The underlined part is the human-TSLPR
extracellular domain, and the italicized
part is linker-human Fc-tag.

### Example 2. Purification of TSLP and TSLP Receptor (TSLPR) Recombinant Proteins

### 2.1 Purification of His-Tagged TSLP Recombinant Proteins of Each Species

The cell expression supernatant samples were centrifuged at high speed to remove impurities and filtered. Nickel columns were equilibrated with PBS solution and washed with 10 times of the column volume. The filtered supernatant samples were applied to the columns. The columns were washed with PBS solution containing 30 mM imidazole until the A₂₈₀ reading dropped to baseline. The target proteins were then eluted with PBS solution containing 300 mM imidazole, and the elution peaks were collected. The proteins were concentrated and exchanged into PBS, and aliquoted for use after being identified as correct by LC-MS. Obtained were his-tagged human TSLP and cyno TSLP.

### 2.1 Purification of Human Fc-Tagged TSLP of Each Species and Human TSLP Receptor Extracellular Domain Recombinant Proteins

The cell expression supernatant samples were centrifuged at high speed to remove impurities. The recombinant antibody expression supernatant was purified by Protein A columns. The columns were washed with PBS until the A280 reading dropped to baseline. The target proteins were eluted with 100 mM acetate buffer pH 3.5, and neutralized with 1 M Tris-HCl pH 8.0. The obtained proteins were concentrated and exchanged into new solution, and aliquoted for use after being identified as correct by electrophoresis and LC-MS.

### Example 3. Construction and Identification of Recombinant TSLP Receptor and IL7Rα Receptor Cell Lines

In order to screen antibodies that can block TSLP from binding to TSLP receptor, CHO-K1 and BaF3 cell lines simultaneously expressing both human TSLP receptor and human IL7Rα (TSLPR/IL7Rα) were constructed. Lentivirus was used to package the target gene TSLPR/IL7 Rα and cloned into the target cell lines to form stable high-expressing cell lines. Firstly, human TSLPR and human IL7Rα genes were cloned into the plasmids pCDH-CMV-MCS-EF1-puro and pCDH-CMV-MCS-EF1-Neo (SBI, CD500B-1) respectively. Then the lentivirus infection method was used to insert human TSLPR into CHO-K1 and BaF3 cell lines, which were cultured under the selection pressure of 10 μg/ml puromycin (Gibco, US) for three weeks. Then, the second round of infection was carried out. The human IL7Rα gene was inserted into the cell lines and screened with 1 mg/ml G418 (Gibco, US) and 10 μg/ml puromycin for two to three weeks. Finally, CHO-K1 and BaF3 monoclonal cell lines with simultaneous high expression of TSLPR and IL7Rα were screened out by the flow sorting method.

### Example 4. Preparation and Screening of Anti-Human TSLP Antibodies

Anti-human TSLP monoclonal antibodies were produced by immunizing laboratory SJL white mice, female, 6-8 weeks old (Beijing Charles River Laboratory Animal Technology Co., Ltd., animal production license number: SCXK (Beijing) 2012-0001). Housing environment: SPF level. After the mice were purchased, they were kept in a laboratory environment for 1 week, with 12/12 hours light/dark cycle adjustment, temperature 20-25° C.; humidity 40-60%. Mice that had adapted to the environment were immunized with recombinant proteins huTSLP-Fc (25 μg), huTSLP-his (12.5 μg) and cyno TSLP-his (12.5 μg) and TiterMax, Alum or CpG adjuvant. After 4-5 immunizations, mice with high antibody titers in the serum and the titers tending to reach a plateau were selected and sacrificed. The spleen cells were collected and fused with myeloma cells. Splenic lymphocytes and the myeloma cell Sp2/0 cells (ATCC® CRL-8287™) were fused to obtain hybridoma cells by using optimized PEG-mediated fusion steps.

For the initial screening, ELISA binding assays for human and cyno TSLP, assays of blocking human TSLP from binding to its receptor TSLPR, and experiments of inhibiting TSLP-induced proliferation of BaF3 cells were performed. After transferring the hybridoma cells to 24-well plates, the supernatant was re-screened. Hybridoma clones were obtained after two rounds of subcloning of the selected positive clones, and were used for antibody production and purification was performed by affinity methods.

The monoclonal hybridoma cell lines No. 3, No. 119, No. 179 and No. 199 with good activity were obtained after screening, and the hybridoma cells in logarithmic growth phase were collected. RNA was extracted with NucleoZol (MN), and reverse transcription was performed (PrimeScript™ Reverse Transcriptase, Takara, cat #2680A). The cDNA obtained by reverse transcription was amplified by PCR using mouse Ig-Primer Set (Novagen, TB326 Rev.B 0503) and sent to a sequencing company for sequencing. Murine anti-TSLP antibodies were obtained after sequencing: mab3, mab119, mab179 and mab199 sequences, the amino acid sequence of the variable regions thereof are as follows:

>mab3 murine heavy chain variable region sequence:
SEQ ID NO: 6
EVQLQQSGPVLVKPGASVKMSCKASGYTFTDDYMNWVKQSHGKSLEWIGI
ISPYNGGTSYNQKFKGKATLTVDKSSSTAYMELNSLTSEDSAVYYCARED
YDYDGYAMDHWGQGTSVTVSS
>mab3 murine light chain variable region sequence:
SEQ ID NO: 7
QIVLSQSPAILSASPGEKVTMTCRASSSVSYMHWYQQKPGSSPKPWIYAT
SNLASGVPARFSGSGSGTSYSLTISRVEAEDAATYYCQQWSSNRTFGGGT
KLEIK
>mab 119 murine heavy chain variable region
sequence:
SEQ ID NO: 8
QAYLQQSGAELVRPGASVKMSCKASGFAFTTYNMHWVKHTPGQGLEWIGA
IYPGNGETSYNQKFKDRATLTVDKSSRTAYMQLSSLTSEDSAVYFCARED
DYGEGYFDVWGAGTTVTVSS
>mab 119 murine light chain variable region
sequence:
SEQ ID NO: 9
DIVLTQSPASLAVSLGQRATISCRASESVDNSGLSFMHWYQQKPGQPPRL
LLYRASNLGSGIPARFSGSGSGTDFTLTLNPVETDDVATYYCQQINTDPL
TFGAGTKLELK
>mab 179 murine heavy chain variable region
sequence:
SEQ ID NO: 10
QVQLQQSGAELVRPGTSVKVSCKASGYAFTNYLIEWVKQRPGQGLEWIG
VIDPGNGDTNYNENFKGKATLTADKSSSTAYIELSRLTSEDSAVYFCAR
EDNTGTAFDYWGQGTTLTVSS
>mab 179 murine light chain variable region
sequence:
SEQ ID NO: 11
SIVMTQTPKFLLVSAGDRVTISCKASQSVSSDVTWYQQKPGQSPKLLIY
YVSNHYTGVPDRFTGSGYGTDFTFTISSVQAEDLAVYFCQQHHREPLTF
GAGTKLELK
>mab 199 murine heavy chain variable region
sequence:
SEQ ID NO: 12
QVQLQQSGPQLVRPGASVKISCKASGYSFTTYWMHWYKQRPGQGLEWIG
MIDPSDSETTLIQKFKDKATLIVDKSSSTAYMQLSSPTSEDSAVYYCAR
TLDGYYDYWGQGTTLTVSS
>mab 199 murine light chain variable region
sequence:
SEQ ID NO: 13
DIQMTQSPASLSASVGETVTITCRASENIYSYLAWYQQKQGKSPQLLVY
FAKTLAEGVPSRFSGSVSGTQFSLKINSLQPEDFGSYYCQHHYGTPWTF
GGGTKLEIK

The amino acid sequences of the CDR regions obtained according to the Kabat numbering rules are shown in the following table:

TABLE 6
Sequences of heavy chain and light chain CDR
regions of antibodies from hybridoma clones

| Antibody | Heavy chain | | Light chain | |
|---|---|---|---|---|
| mab3 | HCDR1 | DDYMN | LCDR1 | RASSSVSYMH |
| | | SEQ ID | | SEQ ID |
| | | NO: 14 | | NO: 17 |
| | HCDR2 | IISPYNGGTS | LCDR2 | ATSNLAS |
| | | YNQKFKG | | SEQ ID |
| | | SEQ ID | | NO: 18 |
| | | NO: 15 | | |
| | HCDR3 | EDYDYDGYAM | LCDR3 | QQWSSNRT |
| | | DH | | SEQ ID |
| | | SEQ ID | | NO: 19 |
| | | NO: 16 | | |
| mab119 | HCDR1 | TYNMH | LCDR1 | RASESVDNSG |
| | | SEQ ID | | LSFMH |
| | | NO: 20 | | SEQ ID |
| | | | | NO: 23 |
| | HCDR2 | AIYPGNGETS | LCDR2 | RASNLGS |
| | | YNQKFKD | | SEQ ID |
| | | SEQ ID | | NO: 24 |
| | | NO: 21 | | |
| | HCDR3 | EDDYGEGYFD | LCDR3 | QQINTDPLT |
| | | V | | SEQ ID |
| | | SEQ ID | | NO: 25 |
| | | NO: 22 | | |
| mab179 | HCDR1 | NYLIE | LCDR1 | KASQSVSSDV |
| | | SEQ ID | | T |
| | | NO: 26 | | SEQ ID |
| | | | | NO: 29 |
| | HCDR2 | VIDPGNGDTN | LCDR2 | YVSNHYT |
| | | YNENFKG | | SEQ ID |
| | | SEQ ID | | NO: 30 |
| | | NO: 27 | | |
| | HCDR3 | EDNTGTAFDY | LCDR3 | QQHHRFPLT |
| | | SEQ ID | | SEQ ID |
| | | NO: 28 | | NO: 31 |
| mab 199 | HCDR1 | TYWMH | LCDR1 | RASENIYSYL |
| | | SEQ ID | | A |
| | | NO: 32 | | SEQ ID |
| | | | | NO: 35 |
| | HCDR2 | MIDPSDSETT | LCDR2 | FAKTLAE |
| | | LIQKFKD | | SEQ ID |
| | | SEQ ID | | NO: 36 |
| | | NO: 33 | | |
| | HCDR3 | TLDGYYDY | LCDR3 | QHHYGTPWT |
| | | SEQ ID | | SEQ ID |
| | | NO: 34 | | NO: 37 |

Chimeric antibodies were formed by linking the light and heavy chain variable regions of the aforementioned murine antibody with the light and heavy chain constant regions of the human antibody (such as the kappa constant region as shown in SEQ ID NO: 134 and the IgG1-YTE constant region as shown in SEQ ID NO: 133). The chimeric antibody corresponding to clone mab3 was named Ch3, and so forth for other antibodies.

### Example 5. Design of Humanization of Anti-Human TSLP Monoclonal Antibodies

In order to reduce the immunogenicity of murine antibodies, the screened mab3, mab119, mab179 and mab199 antibodies with excellent in vivo and in vitro activities were humanized. Humanization of the murine monoclonal antibodies was performed according to the methods published in many documents in the art. Briefly, human antibody constant domains were used to replace parental (murine antibody) constant domains, human germline antibody sequences were selected according to the homology between the murine and human antibodies, and CDR grafting was performed. Then, based on the three-dimensional structure of the murine antibody, the amino acid residues of VL and VH were subjected to back-mutation, and the constant regions of the murine antibody were replaced with human constant regions, resulting in the final humanized molecule.

### 5.1 Selection and Back Mutations of the Human FR Regions for Mab3

### (1) Selection and Back Mutations of the Human FR Regions

For mab3, the humanized VH template was IGHV1-3*01+IGHJ6*01, and the humanized VL template was IGKV3-20+IGKJ4*01. The CDRs of mab3 were grafted to the human template, and the variable region sequences obtained after grafting are as follows:

hu3 VL-CDR grafted:
SEQ ID NO: 38
EIVLTQSPATLSLSPGERATLSCRASSSVSYMHWYQQKPGQAPRLLI
YATSNLASGIPARFSGSGSGTDFTLTISRLEPEDFAVYYCQQWSSNR
TFGGGTKVEIK
hu3 VH-CDR grafted:
SEQ ID NO: 42
EVQLVQSGAEVKKPGASVKVSCKASGYTFTDDYMNWVRQAPGQRLEW
MGIISPYNGGTSYNQKFKGRVTITRDTSASTAYMELSSLRSEDTAVY
YCAREDYDYDGYAMDHWGQGTTVTVSS

Back mutation design of the mab3 humanized antibody is as shown in the following table:

TABLE 7
Back mutations of the mab3 humanized antibody

| hu3VL | | hu3VH | |
|---|---|---|---|
| hu3VL1 | Grafted | hu3VH1 | Grafted |
| hu3VL2 | L46P, F71Y | hu3VH2 | I69L, R71V, T73K |
| hu3VL3 | L46P, L47W, | hu3VH3 | R38K, M48I, V67A, |
| | I58V, F71Y | | I69L, R71V, T73K |
| hu3VL4 | L46P, L47W, | | |
| | I58V, D70S, F71Y | | |

Note:
Grafted represents grafting the murine antibody CDRs into the human germline FR region sequences. L46P represents that according to the Kabat numbering system, the L at position 46 is mutated back to P.

The sequences of the variable regions of the mab3 humanized antibody are as follows:

>hu3VL1 (hu3 VL-CDR grafted)
SEQ ID NO: 38
EIVLTQSPATLSLSPGERATLSCRASSSVSYMHWYQQKPGQAPRLLIYATSNLA
SGIPARFSGSGSGTDFTLTISRLEPEDFAVYYCQQWSSNRTFGGGTKVEIK
>hu3VL2
SEQ ID NO: 39
EIVLTQSPATLSLSPGERATLSCRASSSVSYMHWYQQKPGQAPRPLIYATSNLA
SGIPARFSGSGSGTDYTLTISRLEPEDFAVYYCQQWSSNRTFGGGTKVEIK
>hu3VL3
SEQ ID NO: 40
EIVLTQSPATLSLSPGERATLSCRASSSVSYMHWYQQKPGQAPRPWIYATSNLA
SGVPARFSGSGSGTDYTLTISRLEPEDFAVYYCQQWSSNRTFGGGTKVEIK
>hu3VL4
SEQ ID NO: 41
EIVLTQSPATLSLSPGERATLSCRASSSVSYMHWYQQKPGQAPRPWIYATSNLA
SGVPARFSGSGSGTSYTLTISRLEPEDFAVYYCQQWSSNRTFGGGTKVEIK
>hu3VH1 (hu3 VH-CDR Grafted)
SEQ ID NO: 42
EVQLVQSGAEVKKPGASVKVSCKASGYTFTDDYMNWVRQAPGQRLEWMGII
SPYNGGTSYNQKFKGRVTITRDTSASTAYMELSSLRSEDTAVYYCAREDYDYD
GYAMDHWGQGTTVTVSS
>hu3VH2
SEQ ID NO: 43
EVQLVQSGAEVKKPGASVKVSCKASGYTFTDDYMNWVRQAPGQRLEWMGII
SPYNGGTSYNQKFKGRVTLTVDKSASTAYMELSSLRSEDTAVYYCAREDYDY
DGYAMDHWGQGTTVTVSS
>hu3VH3
SEQ ID NO: 44
EVQLVQSGAEVKKPGASVKVSCKASGYTFTDDYMNWVKQAPGQRLEWIGII
SPYNGGTSYNQKFKGRATLTVDKSASTAYMELSSLRSEDTAVYYCAREDYDY
DGYAMDHWGQGTTVTVSS

Note:
The single underline represents CDR regions, and the double underline represents back mutation sites.

The aforementioned light and heavy chain variable regions were combined with human germline light and heavy chain constant region sequences to form the final complete light and heavy chain sequences, thus obtaining the antibody with full-length sequence. Exemplarily, for the mab3 humanized antibody in the present disclosure, the heavy chain constant region is the IgG1-YTE constant region shown in SEQ ID NO: 133, and the light chain constant region is the kappa chain constant region shown in SEQ ID NO: 134, but they can also be replaced with other constant regions known in the art.

The sequences of the heavy and light chain variable regions of the obtained mab3 humanized antibodies are shown in the following table:

TABLE 8
Heavy and light chain variable region
sequences of mab3 humanized antibody

| | VH (SEQ | VL (SEQ |
|---|---|---|
| Antibody | ID No.) | ID No.) |
| hu3-01 | 42 | 39 |
| hu3-02 | 42 | 40 |
| hu3-03 | 42 | 41 |
| hu3-04 | 43 | 38 |
| hu3-05 | 43 | 39 |
| hu3-06 | 43 | 40 |
| hu3-07 | 43 | 41 |
| hu3-08 | 44 | 39 |
| hu3-09 | 44 | 40 |
| hu3-10 | 44 | 41 |

The binding activity of mab3 humanized antibody to human TSLP was detected by ELISA method, and the results showed that mab3 humanized antibodies have very good binding ability to human TSLP.

### (2) Point Mutation to hu3 Antibody

It was found by detection that there were hot spots on the MDH sequence of HCDR3 and the NTR sequence of LCDR3 of the mab3 humanized antibody. Therefore, the corresponding hot spots were mutated. The sequences of the CDR regions of the mab3 humanized antibodies obtained after mutation are as follows:

TABLE 9
HCDR3 and LCDR3 sequences after mutation

| | |
|---|---|
| hu3 HCDR3-H110Y | EDYDYDGYAMDY |
| | SEQ ID NO: 45 |
| hu3LCDR3-N93D | QQWSSDRT |
| | SEQ ID NO: 46 |

Note:
The positions of mutation sites in Table 9 are numbered according to the natural order of the variable region sequences.

It can be concluded that the CDR sequences of the mab3 humanized antibody are as follows:

TABLE 10
CDRs after mutation of mab3 humanized antibody

| Heavy chain | | Light chain | |
|---|---|---|---|
| HCDR1 | DDYMN | LCDR1 | RASSSVSYMH |
| | SEQ ID NO: 14 | | SEQ ID NO: 17 |
| HCDR2 | IISPYNGGTSYNQKFKG | LCDR2 | ATSNLAS |
| | SEQ ID NO: 15 | | SEQ ID NO: 18 |
| HCDR3 (general | EDYDYDGYAMDX1 | LCDR3 | QQWSSX2RT |
| formula) | SEQ ID NO: 47 | (general | SEQ ID NO: 48 |
| | | formula 1) | |

Wherein, X₁ is selected from H or Y, X₂ is selected from N or D.

Exemplarily, the CDRs and heavy and light chain variable regions of the humanized antibody hu3-11 obtained after mutation are as follows:

TABLE 11
CDR regions of hu3-11

| Heavy chain | | Light chain | |
|---|---|---|---|
| HCDR1 | DDYMN | LCDR1 | RASSSVSYMH |
| | SEQ ID NO: 14 | | SEQ ID NO: 17 |
| HCDR2 | IISPYNGGTSYNQK | LCDR2 | ATSNLAS |
| | FKG | | SEQ ID NO: 18 |
| | SEQ ID NO: 15 | | |
| HCDR3- | EDYDYDGYAMDY | LCDR3- | QQWSSDRT |
| H110Y | SEQ ID NO: 45 | N93D | SEQ ID NO: 46 |

>Light chain variable region of hu3-11 (hu3 VL4-N93D)
SEQ ID NO: 49
EIVLTQSPATLSLSPGERATLSCRASSSVSYMHWYQQKPGQAPRPWIYATSNLA
SGVPARFSGSGSGTSYTLTISRLEPEDFAVYYCQQWSSDRTFGGGTKVEIK
>Heavy chain variable region of hu3-11 (hu3 VH2-H110Y)
SEQ ID NO: 50
EVQLVQSGAEVKKPGASVKVSCKASGYTFTDDYMNWVRQAPGQRLEWMGII
SPYNGGTSYNQKEKGRVTLTVDKSASTAYMELSSLRSEDTAVYYCAREDYDY
DGYAMDYWGQGTTVTVSS

The light and heavy chain variable regions after hot spot mutation were recombined with human germline light and heavy chain constant region sequences to form complete light and heavy chain sequences, thus obtaining the antibody with full-length sequence.

The binding activity of the antibody obtained after mutation to human TSLP was detected by using ELISA method. The results showed that the affinity activity of hu3-11 to human TSLP is still high, indicating that the hot spot mutations on the HCDR3 and LCDR3 of the mab3 humanized antibody do not affect the activity of antibody.

(3) Affinity Maturation of hu3-11 Antibody

The hu3-11 molecule was subjected to affinity maturation. The process of affinity maturation was as follows:

Construction of the yeast library: degenerate primers were designed, and the designed mutant amino acids were introduced into the antibody hu3-11 scFv mutant libraries by PCR method, with the size of each library of about 10⁹. The constructed yeast libraries were verified for their diversity by sequencing method.

In the first round of screening, about 5×10¹⁰ cells from the hu3-11-scFv mutant libraries and biotinylated TSLP-Fc protein (1-10 μg/ml) were incubated in 50 ml 0.1% bovine serum albumin (BSA)-containing phosphate buffered saline (PBSA) for 1 hour at room temperature. Then, the mixture was washed with 0.1% PBSA to remove unbound antibody fragments. Then, 100 μl of streptomycin beads (Milenyi Biotec, Auburn, CA) were added to the hu3-11-scFv antibody mutant libraries bound to the biotinylated TSLP-Fc, and were loaded on the AutoMACS system for sorting. The cells with high affinity to TSLP-Fc were collected from the antibody library and induced at 250 rpm and 20° C. for 18 h. The obtained enriched library was subjected to the second round of screening against biotinylated recombinant TSLP-Fc protein.

For the third and fourth rounds of screening, the library cells from the previous round were incubated with biotinylated recombinant TSLP-Fc protein (0.1-1 μg/ml) and 10 μg/ml Mouse Anti-cMyc (9E10, sigma) antibody in 0.1% PBSA at room temperature for 1 h. The mixture was washed with 0.1% PBSA to remove unbound antibody fragments. Goat anti-mouse-Alexa488 (A-11001, life technologies) and Streptavidin-PE (5-866, Life technologies) were added and incubated at 4° C. for 1 h. The mixture was washed with 0.1% PBSA to remove unbound antibody fragments. Finally, antibodies with high affinity were screened out by FACS screening (BD FACSAria™ FUSION).

The hu3-11-scFv mutant libraries underwent 2 rounds of MACS screening and 2 rounds of FACS screening by utilizing biotinylated TSLP-Fc antigen. About 400 yeast single clones were then selected for culturing and inducing expression. The binding of yeast single clones to TSLP-Fc antigen was detected by using FACS, and yeast single clones with high affinity were selected and subjected to sequencing verification. The sequenced clones were compared and analyzed. After removing redundant sequences, the non-redundant sequences were converted into full-length antibodies for mammalian cell expression.

The sequences of the light chain variable regions obtained by affinity maturation are as follows:

>hu3VL5
SEQ ID NO: 51
EIVLTQSPATLSLSPGERATLSCRASSSVSYMHWYQQKPGQAPRPWIY
ATSNLASGVPARFSGSGSGTSYTLTISRLEPEDFAVYYCQQSDNVRGF
GGGTKVEIK
>hu3VL6
SEQ ID NO: 52
EIVLTQSPATLSLSPGERATLSCRASSSVSYMHWYQQKPGQAPRPWIY
ATSNLASGVPARFSGSGSGTSYTLTISRLEPEDFAVYYCQQSDSGREF
GGGTKVEIK

The obtained light chain variable regions were recombined with the heavy chain variable regions of the mab3 humanized antibody to obtain a new mab3 humanized antibody. Exemplarily, huVL5 and huVL6 were respectively combined with hu3VH2-H110Y to obtain the new antibody molecules hu3-12 and hu3-13, which in details are as shown follows:

TABLE 12
Antibodies obtained by affinity maturation

| Antibody | hu3VH | hu3VL |
|---|---|---|
| hu3-12 | hu3VH2-H110Y | hu3VL5 |
| hu3-13 | hu3VH2-H110Y | hu3VL6 |

The CDR sequences of the mab humanized antibody obtained after affinity maturation are shown as follows:

TABLE 13
Antibody CDRs of mab3 humanized antibody obtained by affinity maturation

| Antibody | Heavy chain | | Light chain | |
|---|---|---|---|---|
| hu3-12 | HCDR1 | DDYMN | LCDR1 | RASSSVSYMH |
| | | SEQ ID NO: 14 | | SEQ ID NO: 17 |
| | HCDR2 | IISPYNGGTSYNQKFKG | LCDR2 | ATSNLAS |
| | | SEQ ID NO: 15 | | SEQ ID NO: 18 |
| | HCDR3- | EDYDYDGYAMDY | LCDR3-V1 | QQSDNVRG |
| | H110Y | SEQ ID NO: 45 | | SEQ ID NO: 53 |
| hu3-13 | HCDR1 | DDYMN | LCDR1 | RASSSVSYMH |
| | | SEQ ID NO: 14 | | SEQ ID NO: 17 |
| | HCDR2 | IISPYNGGTSYNQKFKG | LCDR2 | ATSNLAS |
| | | SEQ ID NO: 15 | | SEQ ID NO: 18 |
| | HCDR3- | EDYDYDGYAMDY | LCDR3-V2 | QQSDSGRE |
| | H110Y | SEQ ID NO: 45 | | SEQ ID NO: 54 |

The obtained new antibody mab3 humanized antibody was subjected to ELISA to detect its binding activity to human TSLP. The results showed that hu3-12 and hu3-13 still have high binding ability to human TSLP. It showed that changes of LCDR3 would not affect the activity of the hu3 series of antibodies.

In summary, the CDRs of the mab3 humanized antibody have the sequences shown as follows:

TABLE 14
General formula sequences of CDR regions of mab3 humanized antibody

| Heavy chain | | Light chain | |
|---|---|---|---|
| HCDR1 | DDYMN | LCDR1 | RASSSVSYMH |
| | SEQ ID NO: 14 | | SEQ ID NO: 17 |
| HCDR2 | IISPYNGGTSYNQKFKG | LCDR2 | ATSNLAS |
| | SEQ ID NO: 15 | | SEQ ID NO: 18 |
| HCDR3 (general | EDYDYDGYAMDX₁ | LCDR3 (general | QQSDX₃X₄RX ₅ |
| formula) | SEQ ID NO: 47 | formula 2) | SEQ ID NO: 55 |

Wherein, X₁ is H or Y, X₃ is N or S, X₄ is V or G, X₅ is G or E.

The combinations of the antibody heavy and light chain variable regions of the mab3 humanized antibody after hot spot mutation and affinity maturation are shown in the following table:

TABLE 15
Sequences of antibodies after affinity maturation

| | VH (SEQ | VL (SEQ |
|---|---|---|
| Antibody | ID NO) | ID NO) |
| hu3-11 | 50 | 49 |
| hu3-12 | 50 | 51 |
| hu3-13 | 50 | 52 |

### 5.2 Selection and Back Mutations of the Human FR Regions for Mab119

For mab119, IGHV1-69*02 and HJ6*01 were selected as templates for the VH, and IGKV4-1*01 and IGKJ2*01 as well as IGKV3-11*01 and IGKJ2*01 were selected as templates for the VL. The CDR regions of the murine antibody were grafted to the selected humanized templates, and the FR regions were subjected to back mutation to obtain different light chain and heavy chain variable regions. The variable region sequences obtained by CDR grafting are as follows:

>hu119-VL CDR (Grafted, IGKV4-1*01)
SEQ ID NO: 56
DIVMTQSPDSLAVSLGERATINCRASESVDNSGLSFMHWYQQKPGQPP
KLLIYRASNLGSGVPDRFSGSGSGTDFTLTISSLQAEDVAVYYCQQIN
TDPLTFGQGTKLEIK
>hu119VL4 (Grafted, IGKV3-11*01)
SEQ ID NO: 59
EIVLTQSPATLSLSPGERATLSCRASESVDNSGLSFMHWYQQKPGQAP
RLLIYRASNLGSGIPARFSGSGSGTDFTLTISSLEPEDFAVYYCQQIN
TDPLTFGQGTKLEIK
>hu119-VH CDR (Grafted,IGHV1-69*02)
SEQ ID NO: 62
EVQLVQSGAEVKKPGSSVKVSCKASGGTFSTYNMHWVRQAPGQGLEWM
GAIYPGNGETSYNQKFKDRVTITADKSTSTAYMELSSLRSEDTAVYYC
AREDDYGEGYFDVWGQGTTVTVSS

Back mutations of the mab119 humanized antibody are as shown in the following table:

TABLE 16
Back mutations of mab119

| hu119VL | | hu119VH | |
|---|---|---|---|
| hu119VL1 | Grafted (IGKV4-1*01) | hu119VH1 | Grafted (IGHV1-69*02) |
| hu119VL2 | Grafted (IGKV4-1*01) + M4L | hu119VH2 | G27F, I69L, A71V |
| hu119VL3 | Grafted (IGKV4-1*01) + | hu119VH3 | G27F, M48I, |
| | I48L, V58I | | V67A, I69L, A71V |
| hu119VL4 | Grafted | hu119VH4 | G27F, R38K, Q39H, |
| | (IGKV3-11*01) | | M48I, V67A, I69L, A71V |
| hu119VL5 | Grafted (IGKV3-11*01) + | hu119VH5 | M48I, V67A, |
| | A43P, I48L | | I69L, A71V |
| hu119VL6 | Grafted (IGKV3-11*01) + | hu119VH6 | V2A, G27F, M48I, |
| | E1D, A43P, I48L | | V67A, I69L, A71V |
| | | hu119VH7 | M48I, V67A, |
| | | | I69L, A71V, S76R |
| | | hu119VH8 | V2A, G27F, M48I, |
| | | | V67A, I69L, A71V, S76R |

Note:
For example, M4L represents that according to the Kabat numbering system, M at position 4 is mutated back to L. Grafted represents that the murine antibody CDR is implanted into the human germline FR region sequence.

The specific sequences of the variable regions of the mab119 humanized antibody are as follows:

>hu119VL1 (Grafted (IGKV4-1*01))
SEQ ID NO: 56
DIVMTQSPDSLAVSLGERATINCRASESVDNSGLSFMHWYQQKPGQPPKLLIY
RASNLGSGVPDRFSGSGSGTDFTLTISSLQAEDVAVYYCQQINTDPLTFGQGTK
LEIK
>hu119VL2
SEQ ID NO: 57
DIVLTQSPDSLAVSLGERATINCRASESVDNSGLSFMHWYQQKPGQPPKLLIY
RASNLGSGVPDRFSGSGSGTDFTLTISSLQAEDVAVYYCQQINTDPLTFGQGTK
LEIK
>hu119VL3
SEQ ID NO: 58
DIVMTQSPDSLAVSLGERATINCRASESVDNSGLSFMHWYQQKPGQPPKLLLY
RASNLGSGIPDRFSGSGSGTDFTLTISSLQAEDVAVYYCQQINTDPLTFGQGTKL
EIK
>hu119VL4 (Grafted, IGKV3-11*01)
SEQ ID NO: 59
EIVLTQSPATLSLSPGERATLSCRASESVDNSGLSFMHWYQQKPGQAPRLLIYR
ASNLGSGIPARFSGSGSGTDFTLTISSLEPEDFAVYYCQQINTDPLTFGQGTKLEI
K
>hu119VL5
SEQ ID NO: 60
EIVLTQSPATLSLSPGERATLSCRASESVDNSGLSFMHWYQQKPGQPPRLLLYR
ASNLGSGIPARFSGSGSGTDFTLTISSLEPEDFAVYYCQQINTDPLTFGQGTKLEI
K
>hu119VL6
SEQ ID NO: 61
DIVLTQSPATLSLSPGERATLSCRASESVDNSGLSFMHWYQQKPGQPPRLLLYR
ASNLGSGIPARFSGSGSGTDFTLTISSLEPEDFAVYYCQQINTDPLTFGQGTKLEI
K
>hu119VH1 (Grafted)
SEQ ID NO: 62
EVQLVQSGAEVKKPGSSVKVSCKASGGTFSTYNMHWVRQAPGQGLEWMGA
IYPGNGETSYNQKFKDRVTITADKSTSTAYMELSSLRSEDTAVYYCAREDDYG
EGYFDVWGQGTTVTVSS
>hu119VH2
SEQ ID NO: 63
EVQLVQSGAEVKKPGSSVKVSCKASGFTFSTYNMHWVRQAPGQGLEWMGAI
YPGNGETSYNQKFKDRVTLTVDKSTSTAYMELSSLRSEDTAVYYCAREDDYG
EGYFDVWGQGTTVTVSS
>hu119VH3
SEQ ID NO: 64
EVQLVQSGAEVKKPGSSVKVSCKASGFTFSTYNMHWVKHAPGQGLEWIGAI
YPGNGETSYNQKFKDRATLTVDKSTSTAYMELSSLRSEDTAVYYCAREDDYG
EGYFDVWGQGTTVTVSS
>hu119VH4
SEQ ID NO: 65
EVQLVQSGAEVKKPGSSVKVSCKASGFTFSTYNMHWVKHAPGQGLEWIGAI
YPGNGETSYNQKFKDRATLTVDKSTSTAYMELSSLRSEDTAVYYCAREDDYG
EGYFDVWGQGTTVTVSS
>hu119VH5
SEQ ID NO: 66
EVQLVQSGAEVKKPOSSVKVSCKASGGTFSTYNMHWVRQAPGQGLEWIGAI
YPGNGETSYNQKFKDRATLTVDKSTSTAYMELSSLRSEDTAVYYCAREDDYG
EGYFDVWGQGTTVTVSS
>hu119VH6
SEQ ID NO: 67
EAQLVQSGAEVKKPGSSVKVSCKASGFTFSTYNMHWVRQAPGQGLEWIGAI
YPGNGETSYNQKFKDRATLTVDKSTSTAYMELSSLRSEDTAVYYCAREDDYG
EGYFDVWGQGTTVTVSS
>hu119VH7
SEQ ID NO: 68
EVQLVQSGAEVKKPGSSVKVSCKASGGTFSTYNMHWVRQAPGQGLEWIGAI
YPGNGETSYNQKFKDRATLTVDKSTRTAYMELSSLRSEDTAVYYCAREDDYG
EGYFDVWGQGTTVTVSS
>hu119VH8
SEQ ID NO: 69
EAQLVQSGAEVKKPGSSVKVSCKASGETFSTYNMHWVRQAPGQGLEWIGAI
YPGNGETSYNQKFKDRATLTVDKSTRTAYMELSSLRSEDTAVYYCAREDDYG
EGYFDVWGQGTTVTVSS

Note:
The single underline represents variable regions, and the double underline represents back mutations.

The aforementioned light and heavy chain variable regions were combined with human germline light and heavy chain constant region sequences to form the final complete light and heavy chain sequences, thus obtaining the antibody with full-length sequence. Exemplarily, for the mab119 humanized antibody in the present disclosure, the heavy chain constant region is the IgG1-YTE constant region shown in SEQ ID NO: 133, and the light chain constant region is the kappa chain constant region shown in SEQ ID NO: 134, but they can also be replaced with other constant regions known in the art.

The heavy and light chain variable regions of the mab119 humanized antibody are shown in Table 17.

TABLE 17
Heavy and light chain variable regions
of the mab119 humanized antibody

| Antibody | VH (SEQ ID NO) | VL (SEQ ID NO) |
|---|---|---|
| hu119-01 | 62 | 56 |
| hu119-02 | 63 | 56 |
| hu119-03 | 64 | 56 |
| hu119-04 | 65 | 56 |
| hu119-05 | 62 | 57 |
| hu119-06 | 63 | 57 |
| hu119-07 | 64 | 57 |
| hu119-08 | 65 | 57 |
| hu119-09 | 62 | 58 |
| hu119-10 | 63 | 58 |
| hu119-11 | 64 | 58 |
| hu119-12 | 65 | 58 |
| hu119-13 | 64 | 59 |
| hu119-14 | 66 | 59 |
| hu119-15 | 67 | 59 |
| hu119-16 | 68 | 59 |
| hu119-17 | 69 | 59 |
| hu119-18 | 64 | 60 |
| hu119-19 | 66 | 60 |
| hu119-20 | 67 | 60 |
| hu119-21 | 68 | 60 |
| hu119-22 | 69 | 60 |
| hu119-23 | 64 | 61 |
| hu119-24 | 66 | 61 |
| hu119-25 | 67 | 61 |
| hu119-26 | 68 | 61 |
| hu119-27 | 69 | 61 |

The binding activity of the humanized antibody to human TSLP was detected by ELISA method, and the results showed that mab119 humanized antibodies can specifically bind to human TSLP.

(2) Mutations of hu119

It was found by detection that a hot spot was present in the LCDR1 DNS sequence of the mab119 humanized antibody, thus, the corresponding site was mutated to N31S or N31Q. The LCDR1 sequences obtained after mutation are as follows:

TABLE 18
LCDR1 after site mutation of mab119 humanized
antibody

| | |
|---|---|
| hu119 LCDR1-N31S | RASESVDSSGLSFMH |
| | SEQ ID NO: 70 |
| hu119 LCDR1-N31Q | RASESVDQSGLSFMH |
| | SEQ ID NO: 71 |

Note:
The positions of mutation sites in Table 19 are numbered according to the natural order.

Exemplarily, the hu119VL2, hu119VL6 mutant sequences obtained after mutation are as follows:

>hu119V12-N31S
SEQ ID NO: 72
DIVLTQSPDSLAVSLGERATINCRASESVDSSGLSFMHWYQQKPGQPPKLLIYR
ASNLGSGVPDRFSGSGSGTDFTLTISSLQAEDVAVYYCQQINTDPLTFGQGTKL
EIK
>hu119VL2-N31Q
SEQ ID NO: 73
DIVLTQSPDSLAVSLGERATINCRASESVDQSGLSFMHWYQQKPGQPPKLLIY
RASNLGSGVPDRFSGSGSGTDFTLTISSLQAEDVAVYYCQQINTDPLTFGQGTK
LEIK
>hu119VL6-N31S
SEQ ID NO: 74
DIVLTQSPATLSLSPGERATLSCRASESVDSSGLSFMHWYQQKPGQPPRLLLYR
ASNLGSGIPARFSGSGSGTDFTLTISSLEPEDFAVYYCQQINTDPLTFGQGTKLEI
K
>hu119VL6-N31Q
SEQ ID NO: 75
DIVLTQSPATLSLSPGERATLSCRASESYDQSGLSFMHWYQQKPGQPPRLLLYR
ASNLGSGIPARFSGSGSGTDFTLTISSLEPEDFAVYYCQQINTDPLTFGQGTKLEI
K

Note:
The single underline represents variable regions, and the double underline represents back mutations.

The obtained hu119VL2, hu119VL6 mutants were combined with hu119VH to obtain new humanized hu119 antibodies. Exemplarily, hu119VL2-N31S, hu119VL2-N31Q were respectively combined with hu119VH3 to obtain antibodies hu119-28 and hu119-29; hu119VL3-N31S was combined with hu119VH8 to obtain the antibody hu119-30. Exemplary combinations of variable regions of the mutated antibodies are as follows:

TABLE 19
Combinations of the humanized antibody variable
regions after hot spot mutation

| | hu119VH | hu119VL |
|---|---|---|
| hu119-28 | hu119VH3 | hu119VL2-N31S |
| hu119-29 | hu119VH3 | hu119VL2-N31Q |
| hu119-30 | hu119VH8 | hu119VL6-N31S |

The affinity of the antibody obtained after mutation with human TSLP was detected by using ELISA method. The results showed that hu119-28 and hu119-29 antibodies still have relatively high affinity with human TSLP, showing that the N31S and N31Q mutations of LCDR2 will not affect the anti-TSLP antibody activity.

In summary, the CDRs of the mab119 humanized antibody have the sequences shown as follows:

TABLE 20
CDRs of mab 119 humanized antibody

| | | | |
|---|---|---|---|
| HCDR1 | TYNMH | LCDR1- | RASESVDX₆SGLSFMH |
| | SEQ ID NO: 20 | general | SEQ ID NO: 76 |
| | | formula | |
| HCDR2 | AIYPGNGETSYNQKF | LCDR2 | RASNLGS |
| | KD | | SEQ ID NO: 24 |
| | SEQ ID NO: 21 | | |
| HCDR3 | EDDYGEGYFDV | LCDR3 | QQINTDPLT |
| | SEQ ID NO: 22 | | SEQ ID NO: 25 |

Wherein, X₆ is selected from N, S and Q.
   5.3. Humanization of mab179

### (1) Template Selection and Back Mutations for Humanization of Mab179 Murine Antibody

For mab179, IGHV1-69*02 and IGHJ6*01 were selected as templates for the VH, and IGKV4-1*01 and IGKJ2*01 or IGKV2-29*02 and IGKJ2*01 were selected as templates for the VL. The CDR regions of the murine antibody were grafted to the selected humanized templates, and the FR regions were subjected to back mutation to obtain light chain and heavy chain variable regions with different sequences. The humanized variable region sequences and back mutations are as follows:

>hu179VL1 (Graft (IGKV4-1*01))
SEQ ID NO: 77
DIVMTQSPDSLAVSLGERATINCKASQSVSSDVTWYQQKPGQPPKLLIY
YVSNHYTGVPDRFSGSGSGTDFTLTISSLQAEDVAVYYCQQHHRFPLTF
GQGTKLEIK
>hu179VIL (Grafted(IGKV2-29*02))
SEQ ID NO: 81
DIVMTQTPLSLSVTPGQPASISCKASQSVSSDVTWYLQKPGQSPQLLIY
YVSNHYTGVPDRFSGSGSGTDFTLKISRVEAEDVGVYYCQQHHRFPLTF
GQGTKLEIK
>hu179VH1 (Grafted)
SEQ ID NO: 85
EVQLVQSGAEVKKPGSSVKVSCKASGGTFSNYLIEWVRQAPGQGLEWMG
VIDPGNGDTNYNENGKGRVTITADKSTSTAYMELSSLRSEDTAVYYCAR
EDNTGTAFDYWGQGTTVTVSS

TABLE 21
Templates and back mutations for Humanization of mab179

| hu179VL1VL | | hu179VH | |
|---|---|---|---|
| hu179VL1 | Graft (IGKV4-1*01) | hu179VH1 | Graft (IGHV1-69*02) |
| hu179VL2 | Graft (IGKV4-1*01) | hu179VH2 | G27Y, I69L |
| | P43S | | |
| hu179VL3 | Graft (IGKV4-1*01) | hu179VH3 | G27Y, M48I, V67A, |
| | P43S, L73F | | I69L, M80I |
| hu179VL4 | Graft (IGKV4-1*01) | hu179VH4 | G27Y, R38K, M48I, |
| | D1S, P43S | | R66K, V67A, I69L, |
| | | | M80I, S82bR |
| hu179VL5 | Grafted(IGKV2- | hu179VH5 | G27Y, T28A, M48I, |
| | 29*02) | | V67A, I69L |
| hu179VL6 | Graft (IGKV2-29*02), | | |
| | D1S | | |
| hu179VL7 | Graft (IGKV2-29*02) | | |
| | D1S, L73F | | |
| hu179VL8 | Graft (IGKV2-29*02) | | |
| | D1S, S67Y | | |

Note:
For example, P43S represents that according to the Kabat numbering system, P at position 43 is mutated back to S. Grafted represents that the murine antibody CDRs are implanted into the human germline FR region sequences.

The variable regions of the mab179 humanized antibody are shown as follows:

>hu179VL1 (Graft (IGKV4-1*01))
SEQ ID NO: 77
DIVMTQSPDSLAVSLGERATINCKASQSVSSDVTWYQQKPGQPPKLLIYYVSN
HYTGVPDRFSGSGSGTDFTLTISSLQAEDVAVYYCQQHHRFPLTFGQGTKLRIK
>hu179VL2
SEQ ID NO: 78
DIVMTQSPDSLAVSLGERATINCKASQSVSSDVTWYQQKPGQSPKLLIYYVSN
HYTGVPDRFSGSGSGTDFTLTISSLQAEDVAYYYCQQHHRFPLTFGQGTKLEIK
>hu179VL3
SEQ ID NO: 79
DIVMTQSPDSLAVSLGERATINCKASQSVSSDVTWYQQKPGQSPKLLIYYVSN
HYTGVPDRFSGSGSGTDFTFTISSLQAEDVAVYYCQQHHRFPLTFGQGTKLEIK
>hu179VL4
SEQ ID NO: 80
SIVMTQSPDSLAVSLGERATINCKASQSVSSDVTWYQQKPGQSPKLLIYYVSN
HYTGVPDRFSGSGSGTDFTLTISSLQAEDVAVYYCQQHHRFPLTFGQGTKLEIK
>hu179VL5 (Grafted(IGKV2-29*02))
SEQ ID NO: 81
DIVMTQTPLSLSVTPGQPASISCKASQSVSSDVTWYLQKPGQSPQLLIYYVSNH
YTGVPDRFSGSGSGTDFTLKISRVEAEDVGVYYCQQHHRFPLTFGQGTKLEIK
>hu179VL6
SEQ ID NO: 82
SIVMTQTPLSLSVTPGQPASISCKASQSVSSDVTWYLQKPGQSPQLLIYYVSNH
YTGVPDRFSGSGSGTDFTLKISRVEAEDVGVYYCQQHHRFPLTFGQGTKLEIK
>hu179VL7
SEQ ID NO: 83
SIVMTQTPLSLSVTPGQPASISCKASQSVSSDVTWYLQKPGQSPQLLIYYVSNH
YTGVPDRFSGSGSGTDFTFKISRVRAEDVGVYYCQQHHRFPLTFGQGTKLEIK
>hu179VL8
SEQ ID NO: 84
SIVMTQTPLSLSVTPGQPASISCKASQSVSSDVTWYLQKPGQSPQLLIYYVSNH
YTGVPDRFSGSGYGTDFTLKISRVEAEDVGVYYCQQHHRFPLTFGQGTKLEIK
>hu179VH1 (Grafted)
SEQ ID NO: 85
EVQLVQSGAEVKKPGSSVKVSCKASGGTFSNYLIEWVRQAPGQGLEWMGVI
DPGNGDTNYNENFKGRVTITADKSTSTAYMELSSLRSEDTAVYYCAREDNTGT
AFDYWGQGTTVTVSS
>hu179VH2
SEQ ID NO: 86
EVQLVQSGAEVKKPGSSVKVSCKASGYTFSNYLIEWVRQAPGQGLEWMGVI
DPGNGDTNYNENFKGRVTLTADKSTSTAYMELSSLRSEDTAVYYCAREDNTG
TAFDYWGQGTTVTVSS
>hu179VH3
SEQ ID NO: 87
EVQLVQSGAEVKKPGSSVKVSCKASGYTFSNYLIEWVRQAPGQGLEWIGVID
PGNGDTNYNENFKGRATLTADKSTSTAYIELSSLRSEDTAVYYCAREDNTGTA
FDYWGQGTTVTVSS
>hu179VH4
SEQ ID NO: 88
EVQLVQSGAEVKKPGSSVKVSCKASGYTFSNYLIEWVKQAPGQGLEWIGVID
PGNGDINYNENFKGKATLTADKSTSTAYIELSRLRSEDTAVYYCAREDNTGTA
FDYWGQGTTVTVSS
>hu179VHS
SEQ ID NO: 89
EVQLVQSGAEVKKPGSSYKVSCKASGYAFSNYLIEWVRQAPGQGLEWIGVID
PGNGDTNYNENFKGRATLTADKSTSTAYMELSSLRSEDTAVYYCAREDNTGT
AFDYWGQGTTVTVSS

Note:
The single underlined part represents CDRs, and the double underlined represents back mutation sites.

The aforementioned light and heavy chain variable regions were combined with human germline light and heavy chain constant region sequences to form the final complete light and heavy chain sequences, thus obtaining the antibody with full-length sequence. Exemplarily, for the mab199 humanized antibody in the present disclosure, the heavy chain constant region is the IgG1-YTE constant region shown in SEQ TD NO: 133, and the light chain constant region is the kappa chain constant region shown in SEQ TD NO: 134, but they can also be replaced with other constant regions known in the art.

TABLE 22
Combinations of heavy and light chain variable
regions of the mab179 humanized antibody

| Antibody | VH (SEQ ID NO) | VL (SEQ ID NO) |
|---|---|---|
| hu179-01 | 85 | 77 |
| hu179-02 | 85 | 78 |
| hu179-03 | 86 | 77 |
| hu179-04 | 86 | 78 |
| hu179-05 | 87 | 77 |
| hu179-06 | 87 | 78 |
| hu179-07 | 87 | 79 |
| hu179-08 | 87 | 81 |
| hu179-09 | 87 | 82 |
| hu179-10 | 87 | 83 |
| hu179-11 | 87 | 84 |
| hu179-12 | 88 | 77 |
| hu179-13 | 88 | 78 |
| hu179-14 | 89 | 79 |
| hu179-15 | 89 | 80 |
| hu179-16 | 89 | 81 |
| hu179-17 | 89 | 82 |
| hu179-18 | 89 | 83 |
| hu179-19 | 89 | 84 |

The affinity of mab179 humanized antibody with human TSLP was detected by using ELISA method, and the results showed that mab179 humanized antibodies have very good affinity with human TSLP.

(2) Mutations of hu179 Antibody

It was found by detection that there were hot spots on the HCDR2 and LCDR2 sequences of the mab179 humanized antibody. Therefore, the corresponding hot spots were mutated to eliminate the risk of molecule modification.

In one of the embodiments, GNG of HCDR2 of hu179VH1 was subjected to amino acid mutation, and the sequences of hu179VH1 after mutation are:

hu179VH1-N55Q
EVQLVQSGAEVKKPGSSVKVSCKASGGTFSNYLIEWVRQAPGQGLEWMG
VIDPGQGDTNYNENEKGRVTITADKSTSTAYMELSSLRSEDTAVYYCAR
EDNTGTAFDYWGQGTTVTVSS
hu179VH1-N55V
SEQ ID NO: 91
EVQLVQSGAEVKKPGSSVKVSCKASGGTFSNYLIEWVRQAPGQGLEWMG
VIDPGVGDTNYNENFKGRVTITADKSTSTAYMELSSLRSEDTAVYYCAR
EDNTGTAFDYWGQGTTVTVSS
hu179VH1-G56V
SEQ ID NO: 92
EVQLVQSGAEVKKPGSSVKVSCKASGGTFSNYLIEWVRQAPGQGLEWMG
VIDPGNVDTNYNENEKGRVTITADKSTSTAYMELSSLRSEDTAVYYCAR
EDNTGTAFDYWGQGTTVTVSS
Note:
The single underlined part represents CDRs, and the double underlined represents back mutation sites.

The sequences of HCDR2 regions of the mab179 humanized antibody obtained after mutation are as follows:

TABLE 23
HCDR2 mutants of the mab 179 humanized antibody

| | |
|---|---|
| hu179 HCDR2-N55Q | VIDPGQGDTNYNENFKG |
| | SEQ ID NO: 93 |
| hu179 HCDR2-N55V | VIDPGVGDTNYNENFKG |
| | SEQ ID NO: 94 |
| hu179 HCDR2-G56V | VIDPGNVDTNYNENFKG |
| | SEQ ID NO: 95 |

Note:
The positions of mutation sites in Table 24 are numbered according to the natural order.

The CDR regions of the mab179 humanized antibody can be obtained from above, and are shown as follows:

TABLE 24
CDRs of mab 179 humanized antibody after mutation

| | | | |
|---|---|---|---|
| HCDR1 | NYLIE | LCDR1 | KASQSVSSDVT |
| | SEQ ID NO: 26 | | SEQ ID NO: 29 |
| HCDR2 (general | VIDPGX₇X₈DTNYNENFK | LCDR2 | YVSNHYT |
| formula) | G | | SEQ ID NO: 30 |
| | SEQ ID NO: 96 | | |
| HCDR3 | EDNTGTAFDY | LCDR3 | QQHHRFPLT |
| | SEQ ID NO: 28 | | SEQ ID NO: 31 |

Wherein, X₇ is selected from N, Q or V, X₈ is selected from G or V.

The hu179VH1 mutants obtained after mutation were combined with the humanized hu179VL to obtain new mab179 humanized antibodies. Exemplary antibodies of combination of hu179VH1 mutant and hu179VL2 are as follows:

TABLE 25
Combinations of the antibody variable regions after mutation

| Variable region | hu179VH1-N55Q | hu179VH1-N55V | hu179VH1-G56V |
|---|---|---|---|
| hu179VL2 | hu179-20 | hu179-21 | hu179-22 |

The affinity of the antibody obtained after mutation with human TSLP was detected by using ELISA method. The results showed that antibodies after HCDR2 mutation still maintain relatively high affinity with human TSLP. This showed that the N55Q, N55V and G56V point mutations of HCDR2 of the mab179 humanized antibody will basically not affect the affinity activity of the antibody with TSLP.

According to the same method, N55Q, N55V and G56V point mutations (numbered by the natural order) were made on hu179VH2, hu179VH3, hu179VH4 and hu179VH5 respectively, and the heavy and light chain variable regions obtained by mutation were recombined to obtain new mab179 humanized antibodies. Exemplarily, the mutated sequence of hu179VH3 is shown as follows:

>hu179VH3-NSSV
SEQ ID NO: 97
EVQLVQSGAEVKKPGSSVKVSCKASGYTFSNYLIEWVRQAPGQGLEWIG
VIDPGVGDTNYNENEKGRATLTADKSTSTAYIELSSLRSEDTAVYYCAR
EDNTGTAFDYWGQGTTVTVSS

Note: The single underlined part represents CDRs, and the double underlined represents back mutation sites.

In some other examples, LCDR2 of mab179 humanized antibody was subjected to amino acid mutation. Exemplarily, the sequences of hu179VL2 after mutation are as follows:

>hu179VL2-Y50E
SEQ ID NO: 98
DIVMTQSPDSLAVSLGERATINCKASQSYSSDVTWYQQKPGQSPKLLIYEVSN
HYTGVPDRFSGSGSGTDFTLTISSLQAEDVAVYYCQQHHRFPLTFGQGTKLEIK
>hu179VL2-S52D
SEQ ID NO: 99
DIVMTQSPDSLAVSLGERATINCKASQSVSSDVTWYQQKPGQSPKLLIYYVDN
HYTGVPDRFSGSGSGTDFTLTISSLQAEDVAVYYCQQHHRFPLTFGQGTKLEIK
>hu179VL2-S52E
SEQ ID NO: 100
DIVMTQSPDSLAVSLGERATINCKASQSYSSDVTWYQQKPGQSPKLLIYYVEN
HYTGVPDRFSGSGSGTDFTLTISSLQAEDVAVYYCQQHHRFPLTFGQGTKLEIK
>hu179VL2-N53Q
SEQ ID NO: 101
DIVMTQSPDSLAVSLGERATINCKASQSYSSDVTWYQQKPGQSPKLLIYYVSQ
HYTGVPDRFSGSGSGTDFTLTISSLQAEDVAVYYCQQHHRFPLTFGQGTKLEIK
>hu179VL2-N53D
SEQ ID NO: 102
DIVMTQSPDSLAVSLGERATINCKASQSYSSDVTWYQQKPGQSPKLLIYYVSD
HYTGVPDRFSGSGSGTDFTLTISSLQAEDVAVYYCQQHHRFPLTFGQGTKLEIK
>hu179VL2-N53E
SEQ ID NO: 103
DIVMTQSPDSLAVSLGERATINCKASQSVSSDVTWYQQKPGQSPKLLIYYVSE
HYTGVPDRFSGSGSGTDFTLTISSLQAEDVAVYYCQQHHRFPLTFGQGTKLEIK
>hu179VL2-HS4Y
SEQ ID NO: 104
DIVMTQSPDSLAVSLGERATINCKASQSYSSDVTWYQQKPGQSPKLLIYYVSN
YYTGVPDRFSGSGSGTDFTLTISSLQAEDVAVYYCQQHHRFPLTFGQGTKLEIK
>hu179VL2-H54D
SEQ ID NO: 105
DIVMTQSPDSLAVSLGERATINCKASQSVSSDVTWYQQKPGQSPKLLIYYVSN
DYTGVPDRFSGSGSGTDFTLTISSLQAEDVAVYYCQQHHRFPLTFGQGTKLEIK
>hu179VL2-HS4B
SEQ ID NO: 106
DIVMTQSPDSLAVSLGERATINCKASQSYSSDVTWYQQKPGQSPKLLIYYVSN
EYTGVPDRFSGSGSGTDFTLTISSLQAEDVAVYYCQQHHRFPLTFGQGTKLEIK
>hu179VL2-YSSE
SEQ ID NO: 107
DIVMTQSPDSLAVSLGERATINCKASQSYSSDVTWYQQKPGQSPKLLIYYVSN
HETGVPDRFSGSGSGTDFTLTISSLQAEDVAVYYCQQHHFEPLTFGQGTKLEIK

Note: The single underlined part represents CDRs, and the double underlined represents back mutation sites.

The sequences of the mab179 humanized antibody LCDR2 obtained after mutation are as follows:

TABLE 26
LCDR2 mutants of the mab 179 humanized antibody

| Mutant | Sequence |
|---|---|
| hu179 LCDR2-Y50E | EVSNHYT |
| | SEQ ID NO: 108 |
| hu179 LCDR2-S52D | YVDNHYT |
| | SEQ ID NO: 109 |
| hu179 LCDR2-S52E | YVENHYT |
| | SEQ ID NO: 110 |
| hu179LCDR2-N53Q | YVSQHYT |
| | SEQ ID NO: 111 |
| hu179 LCDR2-N53D | YVSDHYT |
| | SEQ ID NO: 112 |
| hu179 LCDR2-N53E | YVSEHYT |
| | SEQ ID NO: 113 |
| hu179 LCDR2-H54Y | YVSNYYT |
| | SEQ ID NO: 114 |
| hu179 LCDR2-H54D | YVSNDYT |
| | SEQ ID NO: 115 |
| hu179 LCDR2-H54E | YVSNEYT |
| | SEQ ID NO: 116 |
| hu179 LCDR2-Y55E | YVSNHET |
| | SEQ ID NO: 117 |

It can be seen from the above that the general formula of LCDR2 of mab179 humanized antibody is: X₉VX₁₀X₁₁X₁₂X₁₃T (SEQ ID NO: 118), wherein X₉ is selected from Y or E, X₁₀ is selected from S, D or E, X₁₁ is selected from N, Q, D or E; X₁₂ is selected from H, Y, D or E; X₁₃ is selected from E or Y. The CDR regions of the mab179 humanized antibody are as shown in the following table:

TABLE 27
CDRs of mab179 humanized antibody

| | | | |
|---|---|---|---|
| HCDR1 | NYLIE | LCDR1 | KASQSVSSDVT |
| | SEQ ID NO: 26 | | SEQ ID NO: 29 |
| HCDR2 | VIDPGX₇X₈DTNYNENFKG | LCDR2 | X₉VX₁₀X₁₁X₁₂X₁₃T |
| | SEQ ID NO: 96 | | SEQ ID NO: 118 |
| HCDR3 | EDNTGTAFDY | LCDR3 | QQHHRFPLT |
| | SEQ ID NO: 28 | | SEQ ID NO: 31 |

Wherein, X₇ is selected from N, Q or V, X₈ is selected from G or V; X₉ is selected from Y or E; X₁₀ is selected from S, D or E; X₁₁ is selected from N, Q, D or E; X₁₂ is selected from H, Y, D or E; X₁₃ is selected from E or Y.

The hu179VL2 mutants obtained after mutation were combined with the humanized hu179 heavy chain variable regions to obtain new mab179 humanized antibodies. As an example, hu179VL2 mutants were combined with hu179VH1, hu179VH3, and the CDRs and the combinations of the heavy and light chain variable regions of the obtained mab179 humanized antibodies are shown as follows:

TABLE 28
The sequences of CDR regions of the mab179 humanized
antibody after LCDR2 mutation

| | | | |
|---|---|---|---|
| HCDR1 | NYLIE | LCDR1 | KASQSVSSDVT |
| | SEQ ID NO: 26 | | SEQ ID NO: 29 |
| HCDR2 | VIDPGNGDTNYNENFKG | LCDR2 | X₅VX₆X₇X₈X₉T |
| | SEQ ID NO: 27 | | SEQ ID NO: 118 |
| HCDR3 | EDNTGTAFDY | LCDR3 | QQHHRFPLT |
| | SEQ ID NO: 28 | | SEQ ID NO: 31 |

Wherein, X₅ is selected from Y or E; X₆ is selected from S, D or E; X₇ is selected from N, Q, D or E; X₈ is selected from H, Y, D or E; X₉ is selected from E or Y.

TABLE 29
Combinations of heavy and light chain variable regions
of mab179 humanized antibody after LCDR2 mutation

| Antibody | VH (SEQ ID NO) | VL (SEQ ID NO) |
|---|---|---|
| hu179-23 | 85 | 102 |
| hu179-24 | 85 | 104 |
| hu179-25 | 87 | 98 |
| hu179-26 | 87 | 99 |
| hu179-27 | 87 | 100 |
| hu179-28 | 87 | 101 |
| hu179-29 | 87 | 103 |
| hu179-30 | 87 | 105 |
| hu179-31 | 87 | 106 |
| hu179-32 | 87 | 107 |

The affinity of the mab179 humanized antibodies obtained after LCDR2 mutation with human TSLP was detected by using ELISA method. The results showed that antibodies obtained after hot spot site mutation to LCDR2 still have relatively good affinity with human TSLP. This showed that hot spot site mutation to LCDR2 will not affect the binding activity of the mab179 humanized antibodies.

According to the same method, N53Q, N53D, N53S, H54Y, Y50E, S52D, S52E, N53E, H54D, H54E, Y55E mutations were made on LCDR2 of hu179VL3, hu179VL4, hu179VL5, hu179VL6, hu179VL7 and hu179VL8. The light chain variable regions and the heavy chain variable regions after mutation were combined to form new mab humanized antibodies. In one embodiment, the sequence of hu179VL8 after mutation is shown as follows:

hu179VL8-N53E:
SEQ ID NO: 119
SIVMTQTPLSLSVTPGQPASISCKASQSVSSDVTWYLQKPGQSPQLLI
YYVSEHYTGVPDRFSGSGYGTDFTLKISRVEAEDVGVYYCQQHHRFPL
TFGQGTKLEIK

hu179VL8-N53E and hu179VH3-N55V obtained by mutation were combined to obtain a new antibody molecule hu179-33, the CDR sequences of which are shown as follows:

TABLE 30
CDR regions of hu179-33 antibody

| | | | |
|---|---|---|---|
| HCDR1 | NYLIE | LCDR1 | KASQSVSSDVT |
| | SEQ ID NO: 26 | | SEQ ID NO: 29 |
| HCDR2- | VIDPGVGDTNYNENFK | LCDR2- | YVSEHYT |
| N55V | G | N53E | SEQ ID NO: 113 |
| | SEQ ID NO: 94 | | |
| HCDR3 | EDNTGTAFDY | LCDR3 | QQHHRFPLT |
| | SEQ ID NO: 28 | | SEQ ID NO: 31 |

The binding activity of antibodies obtained after mutation to human TSLP was detected by Biacore. Exemplary binding activity of antibodies is shown as follows:

TABLE 31
Affinity of hu179-33 with human TSLP

| Antibody | Affinity to huTSLP KD (M) |
|---|---|
| AMG157 | 8.12E−12 |
| hu179-33 | 9.03E−13 |

The results showed that antibody hu179-33 has relatively high specific binding activity to human TSLP. This indicated that point mutations of hot spots on both HCDR2 and LCDR2 will not affect the affinity of the mab179 humanized antibody to human TSLP. It can be seen that in the mab179 humanized antibody molecule, mutations of N55Q, N55V, G56V made on HCDR2, and mutations of N53Q, N53D, N53S, H54Y, Y50E, S52D, S52E, N53E, H54D, H54E, Y55E made on LCDR2 will not affect the binding of the antibody to human TSLP, i.e., will not affect the activity of anti-TSLP antibodies.

### 5.4 Selection and Back Mutations of the Human FR Regions for Mab199 Antibody

For mab199, IGHV1-46*01 and HJ6*01 were selected as templates for the VH, and IGKV1-39*01 and IGKJ4*01 were selected as templates for the VL. The CDR regions of the murine antibody were grafted to the selected humanized templates, and the FR region was subjected to back mutation to obtain light chain and heavy chain variable regions with different sequences. The back mutations are as shown in Table 32.

TABLE 32
Design of back mutations for mab199

| hu199VL | | hu199VH | |
|---|---|---|---|
| hu199VL1 | Grafted | hu199VH1 | Grafted |
| hu199VL2 | I48V | hu199VH2 | R71V, T73K, V78A |
| hu199VL3 | A43S, K45Q, | hu199VH3 | M69L, R71V, T73K, |
| | I48V, D70Q | | V78A |
| hu199VL4 | G66V | hu199VH4 | M48I, V67A, M69L, |
| | | | R71V, T73K, V78A |
| hu199VL5 | I48V, G66V | hu199VH5 | R38K, M48I, R66K, |
| | | | V67A, M69L, R71V, |
| | | | T73K, V78A |
| hu199VL6 | A43S, K45Q, | hu199VH6 | R38K, R66K, R71V, |
| | I48V, G66V, | | T73K, V78A |
| | D70Q | | |
| | | hu199VH7 | R38K, R67K, M69L, |
| | | | R71V, T73K, V78A |

Note:
For example, I48V represents that according to the Kabat numbering system, I at position 48 is mutated back to V. Grafted represents that the murine antibody CDRs are implanted into the human germline FR region sequences.

The variable regions of the mab199 humanized antibody are shown as follows:

>hu199VL1 (Grafted)
SEQ ID NO: 120
DIQMTQSPSSLSASVGDRVTITCRASENIYSYLAWYQQKPGKAPKLLIYFAKTL
AEGVPSRFSGSGSGTDFTLTISSLQPEDFATYYCQHHYGTPWTFGGGTKVEIK
>hu199VL2
SEQ ID NO: 121
DIQMTQSPSSLSASVGDRVTITCRASENIYSYLAWYQQKPGKAPKLLVYFAKT
LAEGVPSRFSGSGSGTDFTLTISSLQPEDFATYYCQHHYGTPWTFGGGTKVEIK
>hu199VL3
SEQ ID NO: 122
DIQMTQSPSSLSASVGDRVTITCRASENIYSYLAWYQQKPGKSPQLLVYFAKT
LAEGVPSRFSGSGSGTQFTLTISSLQPEDFATYYCQHHYGTPWTFGGGTKVEIK
>hu199VL4
SEQ ID NO: 123
DIQMTQSPSSLSASVGDRVTITCRASENIYSYLAWYQQKPGKAPKLLIYFAKTL
AEGVPSRFSGSVSGTDFTLTISSLQPEDFATYYCQHHYGTPWTFGGGTKVEIK
>hu199VL5
SEQ ID NO: 124
DIQMTQSPSSLSASVGDRVTITCRASENIYSYLAWYQQKPGKAPKLLVYFAKT
LAEGVPSRFSGSVSGTDFTLTISSLQPEDFATYYCQHHYGTPWTFGGGTKVEIK
>hu199VL6
SEQ ID NO: 125
DIQMTQSPSSLSASVGDRVTITCRASENIYSYLAWYQQKPGKSPQLLVYFAKT
LAEGVPSRFSGSVSGTQFTLTISSLQPEDFATYYCQHHYGTPWTFGGGTKVEIK
>hu199VH1 (Grafted)
SEQ ID NO: 126
EVQLVQSGAEVKKPGASVKVSCKASGYTFTTYWMHWVRQAPGQGLEWMG
MIDPSDSETTLIQKFKDRVTMTRDTSTSTVYMELSSLRSEDTAVYYCARTLDG
YYDYWGQGTTVTVSS
>hu199VH2
SEQ ID NO: 127
EVQLVQSGAEVKKPGASVKVSCKASGYTFTTYWMHWVRQAPGQGLEWMG
MIDPSDSETTLIQKFKDRVTMTVDKSTSTAYMELSSLRSEDTAVYYCARTLDG
YYDYWGQGTTVTVSS
>hu199VH3
SEQ ID NO: 128
EVQLVQSGAEVKKPGASVKVSCKASGYTFTTYWMHWVRQAPGQGLEWMG
MIDPSDSETTLIQKFKDRVTLTVDKSTSTAYMELSSLRSEDTAVYYCARTLDG
YYDYWGQGTTVTVSS
>hu199VH4
SEQ ID NO: 129
EVQLVQSGAEVKKPGASVKVSCKASGYTFTTYWMHWVRQAPGQGLEWIGM
IDPSDSETTLIQKFKDRATLTVDKSTSTAAMELSSLRSEDTAVYYCARTLDGY
YDYWGQGTTVTVSS
>hu199VH5
SEQ ID NO: 130
EVQLVQSGAEVKKPGASVKVSCKASGYTFTTYWMHWVKQAPGQGLEWIGM
IDPSDSETTLIQKFKDKAKATLTVDKSTSTAYMELSSLRSEDTAVYYCARTLDGY
YDYWGQGTTVTVSS
>hu199VH6
SEQ ID NO: 131
EVQLVQSGAEVKKPGASVKVSCKASGYTFTTYWMHWVKQAPGQGLEWMG
MIDPSDSETTLIQKFKDKVTMTVDKSTSTAYMELSSLRSEDTAVYYCARTLDG
YYDYWGQGTTVTVSS
>hu199VH7
SEQ ID NO: 132
EVQLVQSGAEVKKPGASVKVSCKASGYTFTTYWMHWVKQAPGQGLEWMG
MIDPSDSETTLIQKFKDKVTLTVDKSTSTAYMELSSLRSEDTAVYYCARTLDG
YYDYWGQGTTVTVSS

Note:
The single underlined part represents CDRs, and the double underlined represents back mutation sites.

The aforementioned light and heavy chain variable regions were combined with human germline light and heavy chain constant region sequences to form the final complete light and heavy chain sequences, thus obtaining the antibody with full-length sequence. For the mab199 humanized antibodies, if there is no clear description in the present disclosure, the light chain constant region is the constant region shown in SEQ ID NO: 134, and the heavy chain constant region is the constant region shown in SEQ ID NO: 133.

The obtained mab199 humanized antibodies are shown as follows:

TABLE 33
Sequences of heavy and light chain variable
regions of the mab199 humanized antibodies

| Antibody | VH (SEQ ID NO) | VL (SEQ ID NO) |
|---|---|---|
| hu199-01 | 127 | 120 |
| hu199-02 | 127 | 121 |
| hu199-03 | 127 | 122 |
| hu199-04 | 127 | 123 |
| hu199-05 | 127 | 124 |
| hu199-06 | 127 | 125 |
| hu199-07 | 128 | 120 |
| hu199-08 | 128 | 121 |
| hu199-09 | 128 | 122 |
| hu199-10 | 128 | 123 |
| hu199-11 | 128 | 124 |
| hu199-12 | 128 | 125 |
| hu199-13 | 129 | 120 |
| hu199-14 | 129 | 121 |
| hu199-15 | 129 | 122 |
| hu199-16 | 129 | 123 |
| hu199-17 | 129 | 124 |
| hu199-18 | 129 | 125 |
| hu199-19 | 130 | 120 |
| hu199-20 | 130 | 121 |
| hu199-21 | 130 | 122 |
| hu199-22 | 130 | 123 |
| hu199-23 | 130 | 124 |
| hu199-24 | 130 | 125 |
| hu199-25 | 131 | 120 |
| hu199-26 | 131 | 121 |
| hu199-27 | 131 | 122 |
| hu199-28 | 131 | 123 |
| hu199-29 | 131 | 124 |
| hu199-30 | 131 | 125 |
| hu199-31 | 132 | 120 |
| hu199-32 | 132 | 121 |
| hu199-33 | 132 | 122 |
| hu199-34 | 132 | 123 |
| hu199-35 | 132 | 124 |
| hu199-36 | 132 | 125 |

The activity of the mab199 humanized antibodies blocking the binding of TSLP to TSLP receptor was detected by using ELSA method, and the detection results are as follows:

TABLE 34
Activity of mab199 humanized antibody blocking
the binding of TSLP to TSLP receptor

| Antibody | IC50 (nM) | Antibody | IC50 (nM) | Antibody | IC50 (nM) | Antibody | IC50 (nM) |
|---|---|---|---|---|---|---|---|
| hu199-01 | 0.1912 | hu199-10 | 0.3910 | hu199-191 | 0.6584 | hu199-28 | 0.4619 |
| hu199-02 | 0.2193 | hu199-11 | 0.3648 | hu199-20 | 0.4001 | hu199-29 | 0.5543 |
| hu199-03 | 0.2077 | hu199-12 | 0.3700 | hu199-21 | 0.5353 | hu199-30 | 0.3493 |
| hu199-04 | 0.4242 | hu199-13 | 0.2395 | hu199-22 | 0.3449 | hu199-31 | 0.3044 |
| hu199-05 | 0.4726 | hu199-14 | 0.3112 | hu199-23 | 0.3370 | hu199-32 | 0.2870 |
| hu199-06 | 0.3806 | hu199-15 | 0.2866 | hu199-24 | 0.4960 | hu199-33 | 0.2055 |
| hu199-07 | 0.2834 | hu199-16 | 0.7367 | hu199-25 | 0.2460 | hu199-34 | 0.7107 |
| hu199-08 | 0.2828 | hu199-17 | 0.6111 | hu199-26 | 0.3651 | hu199-35 | 0.4849 |
| hu199-09 | 0.2732 | hu199-18 | 0.4806 | hu199-27 | 0.3544 | hu199-36 | 0.7273 |
| Ch199 | 0.4266 | | | | | | |

The results showed that the mab199 humanized antibodies still have relatively high activity of blocking the binding of TSLP to TSLP receptor.

### 5.5 Antibody Constant Regions

The heavy chain constant region of humanized antibody and chimera antibody can be selected from the group consisting of the constant regions of IgG1, IgG2, IgG4 and variants thereof. Exemplarily, IgG1-YTE constant region was used in the present disclosure, and its sequence is as shown in SEQ TD NO: 133. The light chain constant region can be selected from the light chain constant regions of human κ, λ, chain or variants thereof. Exemplarily, human κ chain constant region was used in the present disclosure, and its sequence is as shown in SEQ ID NO: 134.

>IgG1-YTE heavy chain constant region:
SEQ ID NO: 133
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLYITREPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK
>κ light chain constant region:
SEQ ID NO: 134
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC
Note:
Underlined refers to the designed M252Y, S254T, T256E mutations

The humanized heavy and light chain variable regions in the present disclosure were recombined with the above constant regions to obtain the full-length sequences of the heavy and light chains. Exemplarily, the antibody sequences are as follows:

hu3-13 antibody heavy chain:
SEQ ID NO: 135
EVQLVQSGAEVKKPGASVKVSCKASGYTFTDDYMNWVRQAPGQRLEWMGII
SPYNGGTSYNQKFKGRVTLTVDKSASTAYMELSSLRSEDTAVYYCAREDYDY
DGYAMDYWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLYITREPEVTCVVVDVS
HEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEAL
HNHYTQKSLSLSPGK
hu3-13 antibody light chain:
SEQ ID NO: 136
EIVLTQSPATLSLSPGERATLSCRASSSVSYMHWYQQKPGQAPRPWIYATSNLA
SGVPARFSGSGSGTSYTLTISRLEPEDFAVYYCQQSDSGREFGGGTKVEIKRTVA
APSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC
hu119-30 antibody heavy chain
SEQ ID NO: 137
EAQLVQSGAEVKKPGSSVKVSCKASGFTFSTYNMHWVRQAPGQGLEWIGAI
YPGNGETSYNQKFKDRATLTVDKSTRTAYMELSSLRSEDTAVYYCAREDDYG
EGYFDVWGQGTTVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLYITREPEVTCVVVDVSH
EDPEVKFNWYVDGVEVHNAKTKPREEQYNSTYRVVSVLTYLHQDWLNGKEYKCK
VSNKALPAPIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMHEALH
NHYTQKSLSLSPGK
hu119-30 antibody light chain
SEQ ID NO: 138
DIVLTQSPATLSLSPGERATLSCRASESVDSSGLSFMHWYQQKPGQPPRLLLYR
ASNLGSGIPARFSGSGSGTDFTLTISSLEPEDEAVYYCQQINTDPLTFGQGTKLEI
KRTVAAPSVFIFPPSDEQLKSGTASVVCLLNNFYPREAKVQWKVDNALQSGNSQES
VTEQDSKDSTYSLSSTLTLSKADYEKHKVYACEVTHQGLSSPVTKSFNRGEC
hu179-33 antibody heavy chain
SEQ ID NO: 139
EVQLVQSGAEVKKPGSSVKYSCKASGYTFSNYLIEWVRQAPGQGLEWIGVID
PGVGDTNYNENFKGRATLTADKSTSTAYIELSSLRSEDTAVYYCAREDNTGTA
FDYWGQGTTVTVSSASTKGPSVFPLAPSSKSTSGGTAALGCLVKDYFPEPVTVSW
NSGALTSGVHTPPAVLQSSGLYSLSSVVTVPSSSLGTQTYICNVNHKPSNTKVDKKV
EPKSCDKTHTCPPCPAPELLGGPSVFLFPPKPKDTLYITREPEVTCVVVDVSHEDP
EVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYT
QKSLSLSPGK
hu179-33 antibody light chain
SEQ ID NO: 140
SIVMTQTPLSLSVTPGQPASISCKASQSVSSDVTWYLQKPGQSPQLLIYYVSEH
YTGVPDRFSGSGYGTDFTLKISRVEAEDVGVYYCQQHHRFPLTFGQGTKLEIK
RTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPYTKSFNRGEC
hu199-36 antibody heavy chain
SEQ ID NO: 141
EVQLVQSGAEVKKPGASVKVSCKASGYTFTTYWMHWVKQAPGQGLEWMG
MIDPSDSETTLIQKFKDKVTLTVDKSTSTAYMELSSLRSEDTAVYYCARTLDG
YYDYWGQGTTVTVSSASTKGPSVFPLAPSSKSTSGGTAALGCLVKDYFPEPVTVS
WNSGALTSGVHTFPAVLQSSGLYSLSSVVTVPSSSLQTQTYICNVNHKPSNTKVDKK
VEPKSCDKTHTCPPCPAPELLGGPSVFLFPPKPKDTLYITREPEVTCVVVDVSHED
PEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHY
TQKSLSLSPGK
hu199-36 antibody light chain:
SEQ ID NO: 142
DIQMTQSPSSLSASVGDRVTITCRASENIYSYLAWYQQKPGKSPQLLVYFAKT
LAEGVPSRFSGSVSGTQFTLTISSLQPEDFATYYCQHHYGTPWTFGGGTKVEIK
RTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC

Note:
The underlined part represents CDR, and the italicized part represents constant region.

AMG157 was used as a positive control for the present disclosure, and its sequence is as shown in SEQ ID NO: 143 and SEQ ID NO: 144.

Heavy chain sequence of AMG157
SEQ ID NO: 143
QMQLVESGGGVVQPGRSLRLSCAASGFTFRTYGMHWVRQAPGKGLEWVAV
IWYDGSNKHYADSVKGRFTITRDNSKNTLNLQMNSLRAEDTAVYYCARAPQ
WELVHEAFDIWGQGTMVTVSSASTKGPSVFPLAPCSRSTSESTAALGCLVKD
YFPEPVTVSWNSGALTSGVHTFPAVLQSSGLYSLSSVVTVPSSNFGTQTYTCN
VDHKPSNTKVDKTVERKCCVECPPCPAPPVAGPSVFLFPPKPKDTLMISRTPE
VTCVVVDVSHEDPEVQFNWYVDGVEVHNAKTKPREEQFNSTFRVVSVLTVV
HQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVD
KSRWQQGNVFSCSVMHEALHNHYTQKSLSLSPGK
Light chain sequence of AMG157
SEQ ID NO: 144
SYVLTQPPSVSVAPGQTARITCGGNNLGSKSVHWYQQKPGQAPVLVVYDDS
DRPSWIPERFSGSNSGNTATLTISRGEAGDEADYYCQVWDSSSDHVVFGGGT
KLTVLGQPKAAPSVTLFPPSSEELQANKATLVCLISDFYPGAVTVAWKADSSP
VKAGVETTTPSKQSNNKYAASSYLSLTPEQWKSHRSYSCQVTHEGSTVEKTV
APTECS

In addition, when testing the antibody activity, the present disclosure also used human TSLP receptor and human IL7Rα to construct cell lines, and their sequences are as follows:

Full-length amino acid sequence of human TSLP receptor:
SEQ ID NO: 145
MGRLVLLWGAAVFLLGGWMALGQGGAAEGVQIQIIYFNLETVQVTWNASK
YSRTNLTFHYRENGDEAYDQCTNYLLQEGHTSGCLLDAEQRDDILYESIRNGT
HPVFTASRWMVYYLKPSSPKHVRFSWHQDAVTVTCSDLSYGDLLYEVQYRSP
FDTEWQSKQENTCNVTIEGLDAEKCYSFWVRVKAMEDVYGPDTYPSDWSEV
TCWQRGEIRDACAETPTPPKPKLSKFILISSLAILLMVSLLLLSLWKLWRVKKF
LIPSVPDPKSIFPGLFEIHQGNFQEWITDTQNVAHLHKMAGAEQESGPEEPLVV
QLAKTEAESPRMLDPQTEEKEASGGSLQLPHQPLQGGDVVTIGGFTFVMNDR
SYVAL
Note: The underlined part refers to the signal peptide
Full-length amino acid sequence of human IL7Rα (Uniprot ID: P16871)
SEQ ID NO: 146
MTILGTTFGMVFSLLQVVSGESGYAQNGDLEDAELDDYSFSCYSQLEVNQSQ
HSLTCAFEDPDVNTTNLEFEICGALVEVKCLNFRKLQEIYFIETKKFLLIGKSNI
CVKVGEKSLTCKKIDLTTIVKPEAPFDLSVIYREGANDFVVTENTSHLQKKYV
KVLMHDVAYRQEKDENKWTHVNLSSTKLTLLQRKLQPAAMYEIKVRSIPDHY
FKGFWSEWSPSYYFRTPEINNSSGEMDPILLTISILSFFSVALLVILACVLWKKRI
KPIVWPSLPDHKKTLEHLCKKPRKNLNVSFNPESFLDCQIHRVDDIQARDEVE
GFLQDTFPQQLEESEKQRLGGDVQSPNCPSEDVVITPESFGRDSSLTCLAGNVS
ACDAPILSSSRSLDCRESGKNGPHVYQDLLLSLGTTNSTLPPPFSLQSGILTLNP
VAQGQPILTSLGSNQEEAYVTMSSFYQNQ
Note: The underlined part refers to the signal peptide

The antibodies of the present disclosure can be cloned, expressed and purified using conventional gene cloning and recombinant expression methods.

### TEST EXAMPLES

### Biological Evaluation of In Vitro Activity

### Test Example 1: ELSA Detection of the Binding of Anti-TSLP Antibodies to Human TSLP

Human TSLP-his (SEQ ID NO: 1) was diluted to 1 μg/ml with pH 7.4 PBS (Shanghai BasalMedia, B320) buffer, added at 100 μg/well to 96-well microtiter plates (Corning, CLS3590-100EA) and incubated overnight at 4° C. After discarding the liquid, 200 μl/well of blocking solution with 5% skimmed milk (Bright Dairy skimmed milk powder) diluted in PBS was added and incubated in a 37° C. incubator for 2 hours for blocking. After blocking was over, the blocking solution was discarded, and the plates were washed with PBST buffer (PBS containing 0.1% tween-20, pH 7.4) for 3 times. The antibodies to be tested and the positive antibody AMG157 at different concentrations diluted with the sample diluent were added at 100 μl/well and incubated in a 37° C. incubator for 1 hour. After incubation was over, the plates were washed with PBST for 3 times. HRP-labeled goat anti-mouse secondary antibody (Jackson Immuno Research, 115-035-003) diluted with sample diluent was added at 100 μl/well and incubated at 37° C. for 1 hour. After washing the plate with PBST for 6 times, 50 μl/well TMB chromogenic substrate (KPL, 52-00-03) was added and incubated at room temperature for 10-15 min, and 50 μl/well 1 M H₂SO₄ was added to stop the reaction. The absorption value was read by using a NOVOStar microplate reader at 450 nm. The EC50 value of the TSLP antibodies binding to TSLP was calculated and the results are shown in the following table.

TABLE 35
Results of binding activity of antibodies to human TSLP

| Antibody | EC50 (nM) |
|---|---|
| Ch3 | 0.4929 |
| hu3-01 | 0.8494 |
| hu3-02 | 0.6285 |
| hu3-03 | 0.5545 |
| hu3-04 | 0.4353 |
| hu3-05 | 0.5168 |
| hu3-06 | 0.594 |
| hu3-07 | 0.3853 |
| hu3-08 | 0.4687 |
| hu3-09 | 0.4941 |
| hu3-10 | 0.3879 |
| hu3-12 | 0.1519 |
| hu3-13 | 0.1477 |
| Ch119 | 0.851 |
| hu119-01 | 0.107 |
| hu119-02 | 0.1938 |
| hu119-03 | 0.1593 |
| hu119-04 | 0.1881 |
| hu119-05 | 0.1445 |
| hu119-06 | 0.2206 |
| hu119-07 | 0.2132 |
| hu119-08 | 0.2015 |
| hu119-09 | 0.1492 |
| hu119-10 | 0.2329 |
| hu119-11 | 0.174 |
| hu119-12 | 0.2034 |
| hu119-13 | 0.3438 |
| hu119-14 | 0.345 |
| hu119-15 | 0.3497 |
| hu119-16 | 0.366 |
| hu119-17 | 0.3515 |
| hu119-18 | 0.3455 |
| hu119-19 | 0.3533 |
| hu119-20 | 0.3412 |
| hu119-21 | 0.3987 |
| hu119-22 | 0.351 |
| hu119-23 | 0.3404 |
| hu119-24 | 0.3446 |
| hu119-25 | 0.3575 |
| hu119-26 | 0.3782 |
| hu119-27 | 0.3347 |
| hu119-28 | 0.2648 |
| hu119-29 | 0.2729 |
| hu119-28 | 0.2648 |
| hu119-29 | 0.2729 |
| Ch179 | 0.2023 |
| hu179-01 | 0.1248 |
| hu179-02 | 0.1697 |
| hu179-03 | 0.138 |
| hu179-04 | 0.1886 |
| hu179-05 | 0.1416 |
| hu179-06 | 0.2188 |
| hu179-07 | 0.4478 |
| hu179-08 | 1.01615 |
| hu179-09 | 0.1573 |
| hu179-10 | 0.19 |
| hu179-11 | 0.1369 |
| hu179-12 | 0.1437 |
| hu179-13 | 0.2011 |
| hu179-14 | 0.2053 |
| hu179-15 | 0.2035 |
| hu179-16 | 0.2287 |
| hu179-17 | 0.218 |
| hu179-18 | 0.2458 |
| hu179-19 | 0.1616 |
| hu179-20 | 0.7077 |
| hu179-21 | 0.9784 |
| hu179-22 | 0.7519 |
| hu179-23 | 0.997 |
| hu179-24 | 0.6358 |
| hu179-25 | 0.1313 |
| hu179-26 | 0.2006 |
| hu179-27 | 0.1799 |
| hu179-28 | 0.0906 |
| hu179-29 | 0.2041 |
| hu179-30 | 0.246 |
| hu179-31 | 0.2012 |
| hu179-32 | 0.145 |
| Ch199 | 0.5157 |
| AMG157 | 0.7219 |

The results showed that the antibodies in the present disclosure have very good binding activity with human TSLP.

### Test Example 2: Biacore Detection of the Affinity of Anti-TSLP Humanized Antibodies with Different Species of TSLP

The affinity of the humanized TSLP antibodies to be tested with human and cyno TSLP was detected by a Biacore T200 (GE) instrument.

The molecules to be tested were affinity captured by Protein A biosensor chips (Cat. #29127556, GE). Then the antigens (huTSLP-his, cynoTSLP-his, prepared in Example 1) were allowed to flow across the chip surface, and the reaction signal was detected in real time by using the Biacore T200 instrument to obtain the binding and dissociation curves. After the dissociation of each experimental cycle was completed, the biosensor chips were washed and regenerated with glycine-hydrochloric acid regeneration solution (pH 1.5, Cat. #BR-1003-54, GE). The data was fit with a (1:1) Langmuir model by using BIAevaluation version 4.1, GE software to obtain the affinity value, as shown in the following table.

TABLE 36
Affinity of anti-TSLP antibodies with TSLP of different species

| | affinity to huTSLP | affinity to Cyno TSLP |
|---|---|---|
| Antibody | KD (M) | KD (M) |
| AMG157 | 8.12E−12 | 9.22E−12 |
| hu179-33 | 9.03E−13 | 3.04E−11 |
| hu3-13 | 1.0E−12 | 3.40E−10 |
| hu119-30 | 5.0E−12 | 1.95E−09 |
| hu199-36 | 10.5E−12 | 1.72E−11 |

The results showed that the anti-TSLP antibodies in the present disclosure have relatively high affinity to human TSLP, and can also bind to cyno TSLP.

### Test Example 3: ELSA-Based Experiment of Anti-TSLP Antibodies Blocking the Binding of TSLP to TSLP Receptor

The TSLP receptor has two subunits, TSLPR and IL7R, of which TSLPR is a specific receptor for TSLP, and IL7R is a common receptor for TSLP and IL7. TSLP binds first to TSLPR and then to IL7R. This test example was used to identify whether the TSLP antibodies can block the binding of TSLP to the extracellular domain of recombinant expressed TSLPR receptor protein.

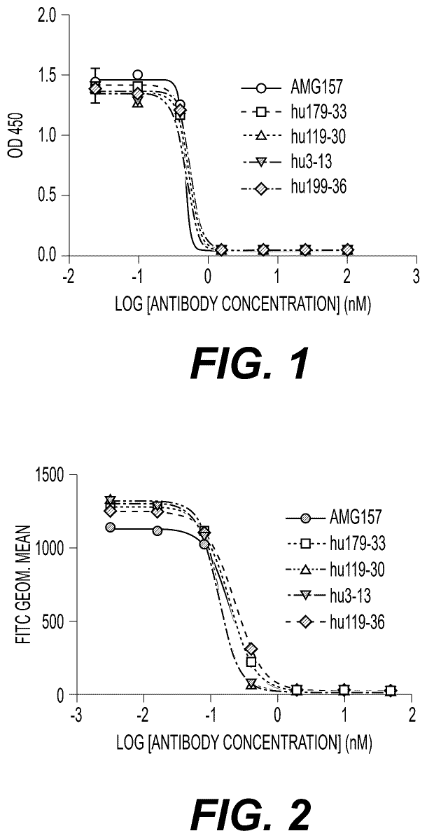

The ELISA plates were coated with human-TSLPR-Fc-ECD (2 μg/ml, SEQ ID NO: 5) and incubated overnight at 4° C. After discarding the liquid, 200 μl/well blocking solution with 5% skimmed milk diluted in PBS was added and incubated in a 37° C. incubator for 2 hours for blocking. After blocking was over, the blocking solution was discarded and the plates were washed with PBST buffer (PBS containing 0.05% tween-20, pH7.4) for 3 times. Biotin-labeled huTSLP-Fc antigen was prepared at 3 nM, and the antibodies to be tested was serially diluted starting from 200 nM. The antigen and antibody were 1:1 mixed, then placed at 37° C. for 15 min, added at 100 μl per well to the microtiter plates and placed at 37° C. for 1 h. The plates were washed with PBST for 3 times. Streptavidin-Peroxidase Polymer diluted to 1:4000 with the sample diluent was added at 100 μl/well and incubated at 37° C. for 1 hour. After washing the plates with PBST for 5 times, 100 μl/well TMB chromogenic substrate (KPL, 52-00-03) was added and incubated at room temperature for 3-10 min, and 100 μl/well 1M H₂SO₄ was added to stop the reaction. The absorption value was read by using a NOVOStar microplate reader at 450 nm. The IC50 value of the TSLP antibodies blocking the binding of TSLP to TSLPR was calculated and the results are shown in Table 37 and FIG. 1.

TABLE 37
Results of blocking activity of antibodies

| Antibody | hu179-33 | hu119-30 | hu3-13 | hu199-36 |
|---|---|---|---|---|
| IC50 (nM) | 0.5038 | 0.5192 | 0.4975 | 0.5693 |

The results showed that all the antibodies of the present disclosure can strongly inhibit the binding of TSLP to its receptor TSLPR.

### Test Example 5: FACS-Based Experiment of TSLP Antibody Blocking the Binding of TSLP to TSLP Receptor

This test example was used to identify whether the anti-TSLP antibodies can respectively block the binding of TSLP to TSLPR/IL7R receptors on the surface of CHOK1 cell line.

The detailed method was: CHOK1-TSLPR/IL7R was cultured with DME/F12 containing 10% FBS, 1 mg/ml G418 and 10 μg/ml puromycin. CHOK1-TSLPR/IL7R cells in good condition were centrifuged (1000 rpm, 5 min), washed once with 2% FBS in PBS. The cells were counted and adjusted to a cell concentration of 1×10⁶/ml. 50 μl of cells were added to round-bottomed 96-well plates. The antibodies to be tested were diluted with PBS solution containing 2% BSA, with an initial concentration of 20 nM and 8 gradients at a ratio of 1:4. Biotin-labeled TSLP-Fc antigen was prepared at 2 nM. The antigen and antibody were 1:1 mixed and placed at 37° C. for 15 min. The mixture was added at 50 μl per well to the 96-well plates where the cells have been plated, and incubated at 4° C. for 1 hour. After incubation was over, the plates were centrifuged at 4° C. (800 g, 5 min) and the supernatant was discarded. The plates were washed twice with 200 μl of pre-cooled PBS by centrifugation. 1:1000 diluted PE-SA secondary antibody was added and incubated at 4° C. in the dark for 40 min. Then the plates were centrifuged at 4° C. (800 g, 5 min) and the supernatant was discarded. 200 μl of pre-cooled PBS was added to blow up the cells, which were washed by centrifugation at 4° C. for three times. 100 μl PBS was added and the plate was loaded onto the machine for plate reading. The IC50 value of TSLP antibodies blocking the binding of TSLP to TSLPR/IL7R was calculated according to the value of the fluorescence signals. The results are as shown in Table 38.

TABLE 38
Results of antibodies blocking cell surface TSLPR

| Antibody | AMG157 | hu179-33 | hu119-30 | hu3-13 | hu199-36 |
|---|---|---|---|---|---|
| IC50 (nM) | 0.2068 | 0.1867 | 0.1368 | 0.1325 | 0.2270 |

The results showed that the antibodies of the present disclosure can all relatively strongly block the binding of TSLP to cell surface TSLPR/IL7R.

### Test Example 6: Anti-TSLP Antibodies Inhibited TSLP-Induced Chemokine Production

TSLP can induce the naive myeloid dendritic cells (mDCs) to be matured and to secrete the chemokine thymus activation regulatory chemokine (TARC) and osteoprotegerin (OPG), thereby further mediating the innate and adaptive immune inflammatory response. This test example was used to verify that the obtained antibodies can block TSLP-induced chemokine production by mDCs, thereby blocking the occurrence of innate and adaptive inflammation response.

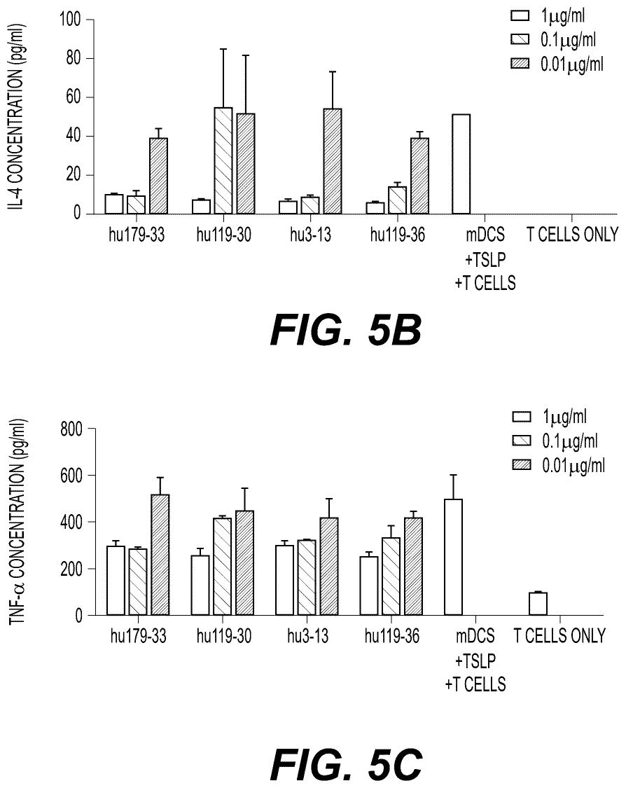

Naive myeloid mDCs were separated and purified from human peripheral blood mononuclear cells (PBMCs) by using magnetic bead sorting method (CD1c (BDCA-1)+Dendritic Cell Isolation Kit, Miltenyi Biotec). The obtained mDCs were seeded in 96-well cell culture plates. Serially diluted antibody samples and human TSLP (huTSLP-his, final concentration of 50 ng/ml) were pre-incubated for about 45 minutes (37° C.) and then respectively added to each cell culture well containing mDCs to stimulate mDCs in vitro. The plates were placed in an incubator to culture for 48 hours. The cell culture supernatant was collected and diluted properly, and then the chemokine content therein was detected by using ELISA method. TARC was detected by using human CCL17/TARC Quantikine ELISA Kit from R&D Company; OPG content was detected by using human CCL22/MDC Quantikine ELISA Kit (R&D), and the results are shown in FIG. 4A-FIG. 4B.

The results showed that all the antibodies in the present disclosure can significantly inhibit TSLP-induced TARC and OPG chemokine production, indicating that the antibodies in the present disclosure can block the occurrence of innate and adaptive inflammatory response.

### Test Example 7. Anti-TSLP Antibodies Blocked the Proliferation of BaF3-TLSPR/IL7R Cells Induced by Native TSLP

BaF3-hTSLPR/hIL7R cells can proliferate under the stimulation of native TSLP. Binding of antibodies to native TSLP reduces the stimulatory effect of TSLP on BaF3-hTSLPR/hIL7R cells.

NHLF cells (BeNa Culture Collection BNCC340764) and HLF1 cells (BeNa Culture Collection BNCC337730) were cultured until the cells grew to 80%, and the supernatant was discarded. Human lung fibroblasts, NHLF (BeNa Culture Collection BNCC340764) and HLF1 (BeNa Culture Collection BNCC337730), were stimulated with 10 ng/ml human IL1-β (Sino Biological GMP-10139-HNAE), 20 ng/ml IL13 (R&D 213-ILB-005), 20 ng/ml TNF-α (PEPROTECH 300-01A) for 72 hours to induce the production of native TSLP. After stimulation was over, the cell supernatant was collected and centrifuged at 4500 rpm for 5 min to remove cell debris. The supernatant was collected, concentrated for about 10 times by concentration columns, and filtered for later use.

BaF3-hTSLPR/hIL17R cells were cultured in RPMI1640 with 10% FBS (10 ng/mL mIL3, R&D 213-ILB-005), adjusted to a density of 1×10⁴ cells/ml and cultured in a 37° C., 5% CO₂ incubator to Logarithmic growth phase. The cells were collected, centrifuged at 800 rpm/min for 5 min, and the supernatant was discarded; the cells were washed with PBS for three times to remove the cytokines that stimulate their proliferation in the culture medium. The cells were resuspended in RPMI1640 medium with 4% FBS, seeded into 96-well plates at 4000 cells/50 μl/well and cultured in an incubator for 2 h. The antibodies to be tested was serially diluted by using native TSLP at 10-fold ratio, with an initial antibody concentration of 100 nM, resulting in 3 dilution gradients, 100 nM, 10 nM and 1 nM. 50 μl/well of the diluted antibody/antigen mixture was added to the cells with the final antibody concentration of 50 nM, 5 nM, 0.5 nM. The plates were incubated in a 37° C., 5% CO₂ incubator for 72 h. Then 30 μL CellTiter-Glo (Promega) was added to each well and incubated in the dark at room temperature for 10 min, and detected by using the Luminescence program with Cytation5 cell imager. The results are shown in the following table.

TABLE 39
Results of anti-TSLP antibodies inhibiting the
proliferation of BaF3-TLSPR/IL7R cells

| Antibody | AMG157 | hu179-33 | hu3-13 | hu119-30 |
|---|---|---|---|---|
| IC50 (nM) | 3.379 | 0.02279 | 0.2888 | 1.533 |

The results showed that all the antibodies obtained in the present disclosure can significantly inhibit the activity of native TSLP to stimulate the proliferation of BaF3, especially hu179-33, the activity of which was more than 100 times of that of AMG157.

### Test Example 8: Experiment of Anti-TSLP Antibodies Inhibiting TSLP-Induced Proliferation of BaF3 Cells Overexpressing TSLPR/IL7R

TSLP can bind to TSLPR/IL7R on the surface of BaF3, thereby promoting the proliferation of BaF3. This test example was used to identify whether the antibodies of the present disclosure can block the activity of TSLP to induce the proliferation of BaF3.

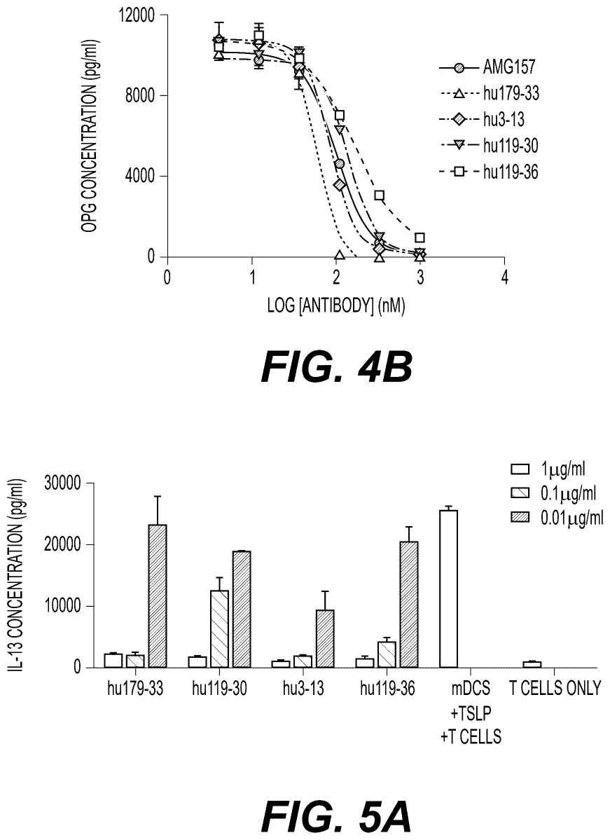

Specifically, BaF3 cells overexpressing TSLPR/IL7R were cultured in RPMI1640 with 10% FBS and 2 ng/mL rhIL3 (MultiSciences, Catalog No. 96-AF-300-03-20), cultured in a 37° C., 5% CO₂ incubator, with the cell density not exceeding 1×10⁶ cells/ml. When detecting the antibodies, cells in logarithmic growth phase were washed with PBS for three times and centrifuged at 800 rpm for 5 min. The cell density was adjusted to 8000 cells/well/90 μl with RPMI1640 (2% FBS, recombinant human TSLP-Fc: 40 ng/ml). 10 μl of serially diluted antibody to be tested was added to the 96-well plates and cultured for 2 days. 30 μl cell titer was added and mixed for detection. IC50 was calculated according to the reading. The results are shown in Table 40 and FIG. 3.

TABLE 40
Inhibition of the proliferation activity of BaF3 cells by antibodies

| Antibody | AMG157 | hu179-33 | hu119-30 | hu3-13 | hu199-36 |
|---|---|---|---|---|---|
| IC50 (nM) | 0.5730 | 0.4092 | 0.4305 | 0.4436 | 0.4760 |

The results showed that all the antibodies of the present disclosure have relatively strong ability to inhibit TSLP-mediated proliferation of BaF3 cells.

### Test Example 9: Humanized Anti-TSLP Antibodies Blocked TSLP-Induced Differentiation of Native CD4⁺ T Cells into Th2 Cells

TSLP can induce the maturation of primary myeloid mDC cells. Mature mDC cells highly express OX40 ligand, which can bind to OX40 on the surface of native CD4⁺ T cells, thereby differentiating the native CD4⁺ T into Th2 cells, which produce factors related to immune response such as IL4/IL5/IL13, etc., leading to Th2 inflammatory response in the body. This test example was used to detect whether the antibodies obtained in the present disclosure can block TSLP-induced differentiation of Th2 cells.

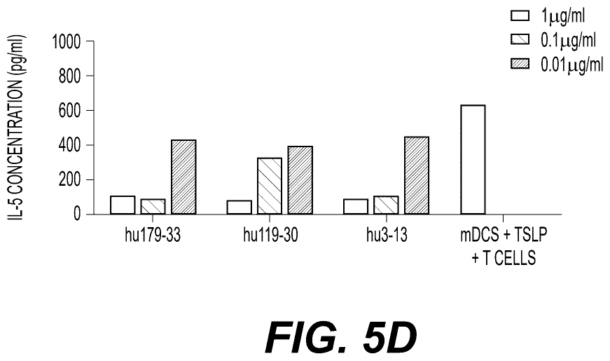

Naive myeloid DCs were separated and purified from human peripheral blood mononuclear cells (PBMCs) by using magnetic bead sorting method (CD1c (BDCA-1)+Dendritic Cell Isolation Kit, Miltenyi Biotec). The obtained mDCs were seeded in 96-well cell culture plates. Serially diluted antibody samples and recombinant expressed human TSLP (huTSLP-his, final concentration 50 ng/ml) were pre-incubated (37° C.) for about 45 minutes and then respectively added to each cell culture well containing mDCs and cultured at 37° C. for 24 hours. The mature mDCs after stimulation were collected and washed with PBS twice. CD4⁺CD45RA⁺ native T cells were extracted from PBMCs by magnetic bead separation method (Myltenyi, Biotec). The native T cells obtained by separation and the mature mDCs were mixed and seeded in 96-well cell culture plates at a ratio of 5:1, and co-cultured for 6 days. The cells were collected and seeded in 96-well plates pre-coated with anti-CD3 (10 μg/ml), and anti-CD28 (1 μg/mL) was added to stimulate the differentiated T cells again. The cells were cultured for 24 hours and finally the cell culture supernatant was collected. Th2-related cytokines secreted by cells in the supernatant were detected by ELISA. TL-4 and TL-5 cytokines were detected by ELISA kits from R&D, and TNF-α and IL-13 were detected by ELISA kits from NeoBioscience. The results are shown in FIG. 5A-FIG. 5D.

The results showed that the antibodies obtained in the present disclosure can significantly inhibit the production of Th2 cytokines IL4, IL5, IL13 and TNF-α, indicating that the antibodies obtained in the present disclosure can block TSLP-induced differentiation of Th2 cells.

What is claimed is:

1. A nucleic acid encoding an anti-TSLP antibody wherein the anti-TSLP antibody comprises a heavy chain variable region and a light chain variable region, wherein: the heavy chain variable region comprises HCDR1, HCDR2 and HCDR3 as shown in SEQ ID NO: 26, SEQ ID NO: 94 and SEQ ID NO: 28, respectively, and the light chain variable region comprises LCDR1, LCDR2 and LCDR3 as shown in SEQ ID NO: 29, SEQ ID NO: 113 and SEQ ID NO: 31, respectively.

2. The nucleic acid according to claim 1, wherein the anti-TSLP antibody is a murine antibody, chimeric antibody, or a humanized antibody.

3. The nucleic acid according to claim 1, wherein the amino acid sequence of the heavy chain variable region comprises the sequence of SEQ ID NO: 97, and the amino acid sequence of the light chain variable region comprises the sequence of SEQ ID NO: 119.

4. The nucleic acid according to claim 1, wherein the anti-TSLP antibody further comprises an antibody heavy chain constant region and a light chain constant region; wherein the heavy chain constant region is selected from the group consisting of human IgG1, IgG2, IgG3 and IgG4 constant regions; and wherein the light chain constant region is selected from the group consisting of human antibody κ (kappa), human antibody λ (lambda) chain constant regions.

5. The nucleic acid according to claim 1, wherein the anti-TSLP antibody comprises a heavy chain constant region as shown in SEQ ID NO: 133, and a light chain constant region as shown in SEQ ID NO: 134.

6. The nucleic acid according to claim 1, wherein the anti-TSLP antibody comprises a heavy chain comprising the amino acid sequence as shown in SEQ ID NO: 139, and a light chain comprising the amino acid sequence as shown in SEQ ID NO: 140.

7. An expression vector comprising a nucleic acid encoding an anti-TSLP antibody wherein the anti-TSLP antibody comprises a heavy chain variable region and a light chain variable region, wherein: the heavy chain variable region comprises HCDR1, HCDR2 and HCDR3 as shown in SEQ ID NO: 26, SEQ ID NO: 94 and SEQ ID NO: 28, respectively, and the light chain variable region comprises LCDR1, LCDR2 and LCDR3 as shown in SEQ ID NO: 29, SEQ ID NO: 113 and SEQ ID NO: 31, respectively.

8. The expression vector according to claim 7, wherein the amino acid sequence of the heavy chain variable region comprises the sequence of SEQ ID NO: 97, and the amino acid sequence of the light chain variable region comprises the sequence of SEQ ID NO: 119.

9. The expression vector according to claim 7, wherein the anti-TSLP antibody comprises a heavy chain comprising the amino acid sequence as shown in SEQ ID NO: 139, and a light chain comprising the amino acid sequence as shown in SEQ ID NO: 140.

10. A host cell comprising the nucleic acid according to claim 1, or the expression vector according to claim 7.

11. The host cell according to claim 10, wherein the host cell is a bacterial cell, a fungal cell, an insect cell or a mammalian cell.

12. The host cell according to claim 10, wherein the host cell is a Chinese Hamster Ovary (CHO) cell or a CHO-K1 cell.
